(19) United States
(12) Patent Application Publication
(10) Pub. No.: US 2023/0250416 A1
(43) Pub. Date: Aug. 10, 2023

(54) INTRON-ENCODED EXTRANUCLEAR TRANSCRIPTS FOR PROTEIN TRANSLATION, RNA ENCODING, AND MULTI-TIMEPOINT INTERROGATION OF NON-CODING OR PROTEIN-CODING RNA REGULATION

(71) Applicants: HELMHOLTZ ZENTRUM MÜNCHEN - DEUTSCHES FORSCHUNGSZENTRUM FÜR GESUNDHEIT UND UMWELT (GMBH), Neuherberg (DE); KLINIKUM RECHTS DER ISAR DER TECHNISCHEN UNIVERSITÄT MÜNCHEN, Munchen (DE)

(72) Inventors: Gil Gregor WESTMEYER, Munchen (DE); Dong-Jiunn Jeffery TRUONG, Freising (DE)

(73) Assignees: HELMHOLTZ ZENTRUM MÜNCHEN - DEUTSCHES FORSCHUNGSZENTRUM FÜR GESUNDHEIT UND UMWELT (GMBH), Neuherberg (DE); KLINIKUM RECHTS DER ISAR DER TECHNISCHEN UNIVERSITÄT MÜNCHEN, Munchen (DE)

(21) Appl. No.: 18/004,292

(22) PCT Filed: Jul. 6, 2021
(86) PCT No.: PCT/EP2021/068659

§ 371 Date: Jan. 4, 2023

(30) Foreign Application Priority Data
Jul. 6, 2020 (EP) 20184281.2
Jul. 6, 2020 (LU) LU101926

(51) Int. Cl. C12N 15/10

(52) U.S. Cl. CPC C12N 15/1055; C12N 15/1082

(57) ABSTRACT

The present invention relates to a method for detecting a nucleic acid construct or part thereof and/or for detecting the expression product of the nucleic acid construct or part thereof, wherein the method comprises inserting a nucleic acid construct or part thereof into an intron or a synthetic intron, wherein the nucleic acid construct comprises certain defined structures according to the present invention. The present invention also relates to the various uses of the method described herein, to the nucleic acid construct, a vector comprising said nucleic acid construct, a cell comprising said nucleic acid construct and/or said vector, and a respective kit.

This application contains a Sequence Listing in a computer readable form, which is incorporated herein by reference.

### TECHNICAL FIELD OF THE INVENTION

The present invention relates to a method for detecting a nucleic acid construct or part thereof and/or for detecting the expression product of the nucleic acid construct or part thereof, wherein the method comprises inserting a nucleic acid construct or part thereof into an intron or a synthetic intron, wherein the nucleic acid construct comprises: a) at least one heterologous nucleic acid sequence, which does not encode a protein; at least one nucleic acid sequence for transcription of the nucleic acid construct or part thereof, and at least one nucleic acid sequence for exporting the nucleic acid construct out of the nucleus, or b) at least one heterologous nucleic acid sequence, which encodes a protein, at least one nucleic acid sequence for transcription of the nucleic acid construct or part thereof, at least one nucleic acid sequence for preventing degradation of the nucleic acid construct or part thereof, at least one nucleic acid sequence for exporting the nucleic acid construct out of the nucleus or part thereof and at least one nucleic acid sequence for translation of the nucleic acid construct or part thereof and at least one nucleic acid sequence for exporting the nucleic acid construct out of the cell. Thus, said nucleic acid construct remains stable after transcription and is exported out of the nucleus and optionally out of the cell, where it can be detected or optionally translated into protein. The nucleic acid construct can be any sequence suitable for the purposes described herein and comprises protein-coding and not protein-coding RNA (e.g., enzymatically active). The present invention also relates to the various uses of the method described herein, to the nucleic acid construct, a vector comprising said nucleic acid construct, a cell comprising said nucleic acid construct and/or said vector, and a respective kit.

### BACKGROUND ART

State of the art techniques for single cell gene expression analysis rely mostly on RNA FISH (fluorescence in situ hybridisation, e.g., FIG. 2h). It enables to detect nucleotide sequences in cells, tissue sections, and even whole tissues. This method is based on the complementary binding of a nucleotide probe to a specific target sequence of DNA or RNA. The probes can be labeled with different reporter bases (Jensen review, 2014) and enable also the detection of RNA in living cells (Bao et al., 2014). However this technique is only reporting the gene expression of a cell at a single, given time point and is not able to dynamically depend on the metabolism of that cell. But such a dynamic metabolic interaction would enable a precisely targeted treatment of pathologic events and thus would be highly desirable. Furthermore, enabling a comprehensive study of dynamic processes, transitions in cell type and function over time with single-cell resolution remained elusive up to now.

WO 2018/057812 deals with the export of cellular content out of living cells and gives a secretion based approach to monitor cells, but fails in influencing the cell chemistry and metabolism and thus fails to represent an alternative treatment technique (e.g., gene-specific intervention into the cell function).

WO 2013/158309 describes non-disruptive gene targeting, providing compositions and methods for integrating one or more genes of interest into cellular DNA, without substantially disrupting the expression of the gene at the locus of integration, i.e. the target locus.

New, non-destructive methods are needed to observe cells closely in biological and medical research and thus being able to obtain informations of the same living cell in different conditions and contexts. This includes the genetic and metabolic state of a cell, the cell type, the development and determination of cells and tissues and changes of these qualities over time.

The inventors of the present invention present a unique, non-destructive gene expression analysis technique with various applications. It combines the natural gene expression of the cell with any kind of reporter or effector molecule suitable for the purpose. This is accomplished by integrating a polynucleotide into the intron of a gene or even a synthetic intron (e.g., consisting of splice donor, branch point, splice acceptor) and thereby coupling its transcription and optionally translation to the endogenous gene promoter. By doing so, the transcription and optionally translation of a specific gene of interest can for example a) be monitored (in combination with a non-protein or protein-coding reporter), b) be inhibited (in combination with f.e. a shRNA or a proteinaceous effector), c) lead to the destruction of the whole cell (in combination with a suicide gene or toxic compound), d) increase proliferative signals (in combination with growth factor expression), e) down-regulate the gene expression gradually, and f) help in forward reprogramming and cell determination (in combination with transcription factors). Further, the gained information is time resolved and allows a single cell or living tissue to be monitored non-invasively more than once. Additionally, the mature mRNA of the gene of interest is not modified and thus the natural gene product remains functionally intact. Taken together, this method represents an important therapeutic and analytic tool and enables ground-breaking discoveries in biological and medical research.

As mentioned above, there is a need for refined genetic research tools. The technical problem underlying the present application is thus to comply with these needs. The technical problem is solved by providing the embodiments reflected in the claim, described in the description and illustrated in the examples and figures that follow.

### SUMMARY OF THE INVENTION

The present invention provides a method for minimally invasive insertion, transcription, transport out of the nucleus and detection of a nucleic acid construct (e.g., DNA and/or corresponding RNA or vice versa) that is simultaneously expressed with an endogenous gene of interest (e.g., by the means of sequences having SEQ ID NOs: 1-50 or sequences which are at least 60% or more, e.g., at least 65%, at least 70%, at least 75%, at least 80%, at least 85%, at least 90%, at least 95%, at least 96%, at least 97%, at least 98%, at least 99% or 100% identical to the sequences having SEQ ID NOs: 1-50 described herein). The described nucleic acid construct may be a non-coding RNA or may be translated into protein when containing a heterologous nucleic acid sequence coding for protein and further structural features. In some aspects of the present invention, hidden splice donor/acceptor sites are destroyed.

The present invention relates to a method for detecting a nucleic acid construct or part thereof and/or detecting the expression product of the nucleic acid construct or part thereof, wherein the method comprises inserting a nucleic acid construct or part thereof into an intron or a synthetic intron, wherein the nucleic acid construct comprises:
a. at least one heterologous nucleic acid sequence, which does not encode a protein;
   at least one nucleic acid sequence for transcription of the nucleic acid construct or part thereof, and
   at least one nucleic acid sequence for exporting the nucleic acid construct or part thereof out of the nucleus,
or
b. at least one heterologous nucleic acid sequence, which encodes a protein,
   at least one nucleic acid sequence for transcription of the nucleic acid construct or part thereof,
   at least one nucleic acid sequence for preventing degradation of the nucleic acid construct or part thereof,
   at least one nucleic acid sequence for exporting the nucleic acid construct or part thereof out of the nucleus, and
   at least one nucleic acid sequence for exporting the nucleic acid construct out of the cell,
   at least one nucleic acid sequence, encoding information that acts as a sensor or actuator of cellular processes (e.g., as shown in FIG. 2, e.g., 2b), and
      at least one nucleic acid sequence for translation of the nucleic acid construct or part thereof.

In one embodiment of the method of the present invention, the at least one nucleic acid sequence for translation of the nucleic acid construct or part thereof is a nucleic acid sequence for translation of the heterologous nucleic acid sequence.

In a further embodiment of the method of the present invention, the nucleic acid construct or part thereof is under the control of an endogenous promoter of the gene of interest.

In one embodiment of the method of the present invention, the at least one nucleic acid sequence for transcription of the nucleic acid construct or part thereof comprises a splice donor nucleic acid sequence and a splice acceptor nucleic acid sequence. Preferably, the splice donor nucleic acid sequence comprises or consists of SEQ ID NO: 1 (or a sequence which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 1) and/or the splice acceptor nucleic acid sequence comprises or consists of SEQ ID NO: 2 (or a sequence which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 2).

In a further embodiment of the method of the present invention, the at least one nucleic acid sequence for exporting the nucleic acid construct or part thereof out of the nucleus is a viral sequence. Preferably, the viral sequence comprises or consists of CTE according to SEQ ID NO: 3 or SEQ ID NO: 25 or SEQ ID NO: 44 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 3 or 25) and/or comprises or consists of WPRE according to SEQ ID NOs: 4 or 42 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 4 or 42). In some aspects of the present invention, the CTE of the present invention is modified, e.g., with deleted SD/SA.

In another preferred embodiment, nuclear export of the intronic sequence, including unmodified, native introns, can be achieved with a sequence according to SEQ ID NO: 53 or SEQ ID NO 54, which codes for a lariat debranching enzyme (DBR1) that has been catalytically inactivated via a H85A mutation (deadDBR1 or dDBR1). Heterologous expression of dDBR1 can be performed, either by plasmid transfection, viral transduction or programmable nucleases-stimulated insertion into a safe-harbor locus, such as AAVS1 (e.g., as shown in FIG. 15 herein)

In one embodiment of the method of the present invention, the at least one nucleic acid sequence for translation of the nucleic acid construct or part thereof is for translation of the heterologous nucleic acid sequence and is initiated by an internal ribosomal entry site (IRES) and an open reading frame (ORF). Preferably, the internal ribosomal entry site (IRES) is the internal ribosomal entry site of the virus Encephalomyocarditis virus (EMCV) according to SEQ ID NO: 5 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 5) or the internal ribosomal entry site of the Hepatitis C virus (HCV) according to SEQ ID NO: 6 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 6).

In one embodiment of the method of the present invention, the at least one nucleic acid sequence for preventing degradation of the nucleic acid construct or part thereof is a poly-A-tail (e.g., a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 7). Preferably, the poly-A-tail is a synthetic poly-A-tail. More preferably, the synthetic poly-A-tail comprises at least 30 adenosines.

In a further embodiment of the method of the present invention, the at least one nucleic acid sequence for preventing degradation of the nucleic acid construct or part thereof is a polyadenylation signal. Preferably, the polyadenylation signal is a late SV40 polyadenylation signal and a rabbit beta-globin polyadenylation signal. More preferably, the late SV40 polyadenylation signal is mutated to be unidirectional. It is preferred that the polyadenylation signals are integrated in the nucleic acid construct in an antisense direction and that they are enclosed with loxP sites and that after transcription, the inverted polyadenylation signal is not separated from the endogenous gene product. It is even more preferred that after the transcription a Cre recombinase is administered to the transcript to invert the polyadenylation signals into sense direction. In some aspects of the present invention, the intervention is carried out at the DNA level.

In one further embodiment of the method of the present invention, the method is non- or minimally invasive for the expression product of the intron or synthetic intron, such that a native and/or fully functional protein is expressed compared to the protein without insertion of the nucleic acid construct or part thereof.

In a further embodiment of the method of the present invention, the insertion of the nucleic acid construct is with targeted transgene insertion.

In one embodiment of the method of the present invention, the at least one heterologous nucleic acid sequence encodes for a protein-coding RNA, a non-coding RNA, a miRNA, an aptamer, a siRNA, a synthetic RNA sequence that can be acted on, a barcode for extranuclear detection, or an endogenous or synthetic export signal. In some aspects of the present invention, the non-coding RNA code could also encode information that may be acted upon by defined logic operations, e.g., via toehold switches or padlock probes, unlocks a specific motif upon an RNA key, e.g., a guide sequence for Cas9, Cas13 or Cas12a handle (sgRNA (Cas9), crRNA (Cas12a, Cas13), pre-crRNA (Cas12a, Cas13) (e.g., as described by Felletti et al., 2016; Nature Communications volume 7, Article number: 12834).

In a further embodiment of the method of the present invention, the at least one heterologous nucleic acid sequence is detected and enables to detect a specific cell. In one embodiment of the method of the present invention, the at least one heterologous nucleic acid sequence is detected and provides information about the transcriptional regulation of the cell or a time stamp of a cellular process.

In one embodiment of the method of the present invention, the heterologous nucleic acid sequence encodes a protein or enzyme selected from the group consisting of: a fluorescent protein, preferably green fluorescent protein; a bioluminescence-generating enzyme, preferably NanoLuc, NanoKAZ, TurboLuc, Cypridina, Firefly, Renilla luciferase, split luciferase, split APEX2 or mutant derivatives thereof (e.g., iodine importer); an enzyme, which is capable of generating a coloured pigment, preferably tyrosinase or an enzyme of a multi-enzymatic process, more preferably the violacein or betanidin synthesis process, a genetically encoded receptor for multimodal contrast agents, preferably Avidin, Streptavidin or HaloTag or mutant derivatives thereof; an enzyme, which is capable of converting a non-reporter molecule into a reporter molecule, preferably TEV protease and picornaviral proteases, more preferably rhinoviral 3C proteases and polioviral 3C protease, SUMO proteases and mutant derivatives thereof; an enzyme, which is capable of inactivating a toxic compound, preferably blasticidin-S-deaminase, puromycin-N-acetyltransferase, neomycin phosphotransferase, hygromycin phosphotransferase and mutant derivatives thereof, an enzyme, which is capable of converting pro-drug/toxin-mediated toxicity, preferably thymidine kinase and mutant derivatives thereof and a small-molecule sensor protein, preferably calmodulin, troponin C, S100 and mutant derivatives thereof.

In a further embodiment of the method of the present invention, the method further comprises combining the expression of the protein or enzyme encoded by the heterologous nucleic acid sequence to the natural expression of the gene comprising the nucleic acid construct or part thereof by using the same promotor.

In one embodiment of the method of the present invention, the heterologous nucleic acid sequence encodes a resistance gene for cell-toxic compounds. Preferably, the method additionally comprises detecting the survival of the cells comprising the nucleic acid construct or part thereof. More preferably, the resistance gene for cell-toxic compounds is used as a selection marker of the cells comprising the nucleic acid construct or part thereof.

In one embodiment of the method of the present invention, the heterologous nucleic acid sequence encodes a Cas enzyme selected from the group consisting of Cas9, Cas12a, Cas12b, Cas12c, Cas13a, Cas13b, Cas13d, Cas14, CasX, and fusion proteins thereof. In some aspects said Cas (i.e., CRISPR-associated) enzyme, e.g., is selected from the group consisting of: Cas9 (e.g., CRISPR-associated endonuclease Cas9, e.g., having EC:3.1.-.- enzymatic activity and/or SEQ ID NO: 9 or UniProtKB Accession Number/s: Q99ZW2, G3ECR, J7RUA5, A0Q5Y3, J3F2B0, C9X1G5, Q927P4, Q8DTE3, Q6NKI3, A11Q68 or Q9CLT2); Cas12a (e.g., CRISPR-associated endonuclease Cas12a, e.g., having EC:3.1.21.1 and/or EC:4.6.1.22 enzymatic activity and/or UniProtKB Accession Number/s: A0Q7Q2, A0A182DWE3 or U2UMQ6, e.g., U2UMQ6 enzyme and/or its variants/mutants may also be referred to as Cas12a/Cpf1 enzymes and/or is/are the preferred Cas12a enzyme/s for use in a mammalian system); Cas12b (e.g., CRISPR-associated endonuclease Cas12b, e.g., having EC:3.1.-.- enzymatic activity and/or UniProtKB Accession Number/s: T0D7A2, e.g., T0D7A2 enzyme and/or its variants/mutants may have temperature optimum at about 48° C. and/or may be the preferred Cas12b enzyme/s for use in a non-mammalian system and/or in an organism being able to function at a temperature of about 48° C. and/or about 37° C. (e.g., BhCas12b, e.g., having RefSeq Accession Number: WP_095142515.1 and/or BhCas12b v4 mutant/s comprising: K846R and/or S893R and/or E837G substitutions/mutations, e.g., using the numbering of WP_095142515.1; e.g., as reported by Strecker et al., 2019; Nat Commun. 2019 Jan. 22; 10(1):212. doi: 10.1038/s41467-018-08224-4)); Cas12c (e.g., CRISPR-associated protein 12c, e.g., selected from the group consisting of: SEQ ID NO: 34 (Cas12c1), SEQ ID NO: 35 (Cas12c2) and SEQ ID NO: 36 (OspCas12c); e.g., as reported by Yan et al., 2019; Science. 2019 Jan. 4; 363(6422):88-91. doi: 10.1126/science.aav7271. Epub 2018 Dec. 6; Cas13a (e.g., CRISPR-associated endoribonuclease Cas13a, e.g., having EC:3.1.-.- enzymatic activity and/or UniProtKB Accession Number/s: C7NBY4, P0DOC6, U2PSH1, A0A0H5SJ89, PODPB7, E4T0I2 or P0DPB8); Cas13b (e.g., CRISPR-associated protein 13b, e.g., UniProtKB Accession Number/s: E6K398); Cas13d (e.g., CRISPR-associated protein 13d, e.g., UniProtKB Accession Number/s: B0MS50 or A0A1C5SD84); Cas14 (e.g., CRISPR-associated protein Cas14, e.g., GenBank Accession Number/s: QBM02559.1, SUY72868.1, VEJ66719.1, SUY81478.1, SUY85836.1 or STC69301.1); CasX (e.g., UniProtKB Accession Number/s: A0A357BT59); and/or sequences, which are at least 60% or more, e.g., at least 65%, at least 70%, at least 75%, at least 80%, at least 85%, at least 90%, at least 95%, at least 96%, at least 97%, at least 98%, at least 99% or 100% identical to the sequences as described herein (e.g., having the corresponding Cas enzymatic activity/activities), e.g., by the means of SEQ ID NOs or accession numbers and/or fusion proteins thereof. Those Cas9 enzymes may preferably refer to the sequence according to the SEQ ID NO: 9 as depicted herein.

In a further embodiment of the method of the present invention, the heterologous nucleic acid sequence encodes an amino acid, which can be metabolized to an antibiotic or derivative thereof, preferably for inducing a genetic system, more preferably for inducing the genetic Tet-On/Tet-OFF system.

In a further embodiment of the method of the present invention, the heterologous nucleic acid sequence encodes an enzyme of a biosynthesis pathway generating a toxin or a mutant thereof.

In one embodiment of the method of the present invention, the heterologous nucleic acid sequence is a suicide gene or a gene, which induces a cell death cascade.

In a further embodiment of the method of the present invention, the heterologous nucleic acid sequence further comprises a polynucleotide encoding a protein, which functions as an activator of the expression of the gene comprising the nucleic acid construct or part thereof.

In one embodiment of the method of the present invention, the heterologous nucleic acid sequence encodes a transcription factor. Preferably, the transcription factor is used to force or refine determination of a stem cell into a defined mature cell.

In a further embodiment of the method of the present invention, the heterologous nucleic acid sequence encodes a transcriptional regulator or a repressor protein or an intrabody.

In one embodiment of the method of the present invention, the heterologous nucleic acid sequence encodes a protein, which is a hormone or has the function of a hormone.

In a further embodiment of the method of the present invention, the heterologous nucleic acid sequence encodes a protein, which is a receptor, preferably a hormone receptor or a mutant derivate thereof.

In one embodiment of the method of the present invention, the heterologous nucleic acid sequence encodes an affinity domain or tag to bind protein, DNA or RNA. Preferably, the protein affinity domain is used to capture the expression product of the nucleic acid construct or part thereof, more preferably the expression product of the heterologous nucleic acid sequence.

In a further embodiment of the method of the present invention, the heterologous nucleic acid sequence encodes an antibody or antibody fragment. Preferably, the antibody or antibody fragment is used to capture the expression product of the nucleic acid construct or part thereof, preferably the expression product of the heterologous nucleic acid sequence.

In one embodiment of the method of the present invention, the protein or enzyme encoded by the heterologous nucleic acid sequence is for preventing pathological changes within the cell.

In one embodiment of the method of the present invention, the method is for detecting biological functions, preferably the regulation of tissue and cell generation, more preferably the expression of non-coding RNA and activity-dependent gene regulation in theranostic cells used in regenerative medicine.

The present invention also relates to/provides a nucleic acid construct comprising or consisting of any of SEQ ID NOs: 1 to 43 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NOs: 1-50).

It is preferred that such a nucleic acid construct is for use in therapy. It is also preferred that such a nucleic acid construct is for use in the treatment or prevention of cancer.

In a further aspect, the present invention also comprises a vector comprising the nucleic acid construct as described elsewhere herein.

In a further aspect, the present invention also comprises a cell comprising the nucleic acid construct or the vector as described elsewhere herein.

The present invention also relates to the use of the nucleic acid construct, the vector, or the cell as described elsewhere herein for detecting the cell identity, the cell state or the time point of expression of the nucleic acid construct.

In a further aspect, the present invention also comprises the use of the nucleic acid construct, the vector, or the cell as described elsewhere herein for enriching cells.

In a further aspect, the present invention comprises the nucleic acid construct, the vector, or the cell as described elsewhere herein for use in the treatment or prevention of a disease. Preferably, the disease is selected from the group consisting of retinopathies, tauopathies, motor neuron diseases, muscular diseases, neurodevelopmental and neurodegenerative diseases. More preferably, the disease is selected from the group consisting of cystic fibrosis, retinitis pigmentosa, myotonic dystrophy, Alzheimer's disease and Parkinson's disease.

In a further aspect, the present invention also comprises the nucleic acid construct, the vector, or the cell as described elsewhere herein for use in tissue generation, gene therapy and in vitro reprogramming of cells.

The present invention also comprises the nucleic acid construct, the vector, or the cell as described elsewhere herein for use as a medicament.

In a further aspect, the present invention also comprises the use of the nucleic acid construct, the vector, or the cell as described elsewhere herein in tissue engineering or regenerative medicine approaches such as CAR-T cell therapies or engineered beta-cell implantation.

In a further aspect, the present invention also comprises a kit for detecting a nucleic acid construct or part thereof and/or detecting the expression product of the nucleic acid construct or part thereof, wherein the kit comprises:
a. at least one heterologous nucleic acid sequence, which does not encode a protein;
   at least one nucleic acid sequence for transcription of the nucleic acid construct or part thereof, and
   at least one nucleic acid sequence for exporting the nucleic acid construct out of the nucleus,
or
b. at least one heterologous nucleic acid sequence, which encodes a protein,
   at least one nucleic acid sequence for transcription of the nucleic acid construct or part thereof,
   at least one nucleic acid sequence for translation of the nucleic acid construct or part thereof,
   at least one nucleic acid sequence for preventing degradation of the nucleic acid construct or part thereof,
   at least one nucleic acid sequence for exporting the nucleic acid construct or part thereof out of the nucleus,
   at least one nucleic acid sequence for exporting the nucleic acid construct out of the cell, and
      a second vector coding for a guided endonuclease, preferably wherein the endonuclease is selected from the group consisting of Cas9 (e.g., UniProtKB Accession Number/s: Q99ZW2, G3ECR, J7RUA5, A0Q5Y3, J3F2B0, C9X1G5, Q927P4, Q8DTE3, Q6NKI3, A1IQ68 or Q9CLT2; or an amino acid sequence, which is at least 60% or more, e.g., at least 65%, at least 70%, at least 75%, at least 80%, at least 85%, at least 90%, at least 95%, at least 96%, at least 97%, at least 98%, at least 99% or 100% identical thereto), Cas12a (e.g., UniProtKB Accession Number/s: A0Q7Q2 or U2UMQ6 or an amino acid sequence, which is at least 60% or more, e.g., at least 65%, at least 70%, at least 75%, at least 80%, at least 85%, at least 90%, at least 95%, at least 96%, at least 97%, at least 98%, at least 99% or 100% identical thereto), TALENs (e.g., UniProtKB Accession Number/s: A0A3G2M3E1 or A0A3G2M3D9 or an amino acid sequence, which is at least 60% or more, e.g., at least 65%, at least 70%, at least 75%, at least 80%, at least 85%, at least 90%, at least 95%, at least 96%, at least 97%, at least 98%, at least 99% or 100% identical thereto), Zinc-finger nucleases (ZFNs) (e.g., UniProtKB Accession Number/s: Q8GXX7 or an amino acid sequence, which is at least 60% or more, e.g., at least 65%, at least 70%, at least 75%, at least 80%, at least 85%, at least 90%, at least 95%, at least 96%, at least 97%, at least 98%, at least 99% or 100% identical thereto) and meganucleases (e.g., UniProtKB Accession Number/s: A0A158RFF2 or an amino acid sequence, which is at least 60% or more, e.g., at least 65%, at least 70%, at least 75%, at least 80%, at least 85%, at least 90%, at least 95%, at least 96%, at least 97%, at least 98%, at least 99% or 100% identical thereto).

In one embodiment of the kit of the present invention, the at least one nucleic acid sequence for transcription of the nucleic acid construct or parts thereof comprises a splice donor nucleic acid sequence and a splice acceptor nucleic acid sequence; preferably wherein the splice donor nucleic acid sequence comprises or consists of SEQ ID NO: 1 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 1) and/or wherein the splice acceptor nucleic acid sequence comprises or consists of SEQ ID NO: 2 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 2).

In a further embodiment of the kit of the present invention, the at least one nucleic acid sequence for exporting the nucleic acid construct or part thereof out of the nucleus is a viral sequence, preferably comprises or consists of CTE according to SEQ ID NO: 3 or SEQ ID NO: 25 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NOs: 3 or 25) and/or comprises or consists of WPRE according to SEQ ID NOs: 4 or 42 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NOs: 4 or 42).

In one embodiment of the kit of the present invention, the first plasmid further comprises an internal ribosomal entry site (IRES), wherein the at least one nucleic acid sequence for translation of the nucleic acid construct or part thereof is for translation of the heterologous nucleic acid sequence and is initiated by an internal ribosomal entry site (IRES); preferably the internal ribosomal entry site of the virus Encephalomyocarditis virus (EMCV) according to SEQ ID NO: 5 (or a sequence which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 5) or the internal ribosomal entry site of the Hepatitis C virus (HCV) according to SEQ ID NO: 6 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 6); and an open reading frame (ORF).

In a further embodiment of the kit of the present invention, the at least one nucleic acid sequence for preventing degradation of the nucleic acid construct or part thereof is a poly-A-tail, preferably a synthetic poly-A-tail, more preferably wherein the synthetic poly-A-tail comprises at least 30 adenosines.

In one embodiment of the kit of the present invention, the heterologous nucleic acid sequence encodes a protein or enzyme selected from the group consisting of a fluorescent protein, preferably green fluorescent protein, a nanobody which works inside cells (intrabody) and which can be fused to a fluorescent protein; a bioluminescence-generating enzyme, preferably NanoLuc, NanoKAZ, TurboLuc, Cypridina, Firefly, Renilla luciferase or mutant derivatives thereof; an enzyme, which is capable of generating a coloured pigment, preferably tyrosinase or an enzyme of a multi-enzymatic process, more preferably the violacein or betanidin synthesis process; a genetically encoded receptor for multimodal contrast agents, preferably Avidin, Streptavidin or HaloTag or mutant derivatives thereof; an enzyme, which is capable of converting a non-reporter molecule into a reporter molecule, preferably TEV protease and picornaviral proteases, more preferably rhinoviral 3C proteases and polioviral 3C protease, SUMO proteases and mutant derivatives thereof; an enzyme, which is capable of inactivating a toxic compound, preferably blasticidin-S-deaminase, puromycin-N-acetyltransferase, neomycin phosphotransferase, hygromycin B phosphotransferase and mutant derivatives thereof, an enzyme, which is capable of converting pro-drug/toxin-mediated toxicity, preferably thymidine kinase and mutant derivatives thereof and a small-molecule sensor protein, preferably calmodulin, troponin C, S100 and mutant derivatives thereof.

### OVERVIEW OF THE SEQUENCE LISTING

SEQ ID NO: 1 is the DNA sequence depicting a 5′-“split-intron”, i.e., a splice donor (SD) of the present invention, which is an exemplary SD of the present invention derived from a mutant beta globin 1^{st} intron (e.g., as described in U.S. Pat. No. 6,893,840 B2), which can be substituted by a suitable (e.g., homologous) SD, including the unmutated 1^{st} intron of the beta globin.

SEQ ID NO: 2 is the DNA sequence depicting a 3′-“split-intron”, i.e., a splice acceptor (SA) of the present invention, which is an exemplary SA derived from a mutant beta globin 1^{st} intron (e.g., as described in U.S. Pat. No. 6,893,840 B2), which can be substituted by another suitable SA (e.g., homologous), including the unmutated 1^{st} intron; exemplified is the a-->t mutation (i.e., A to T substitution) to remove the SA-like-sequence upstream from the intended SA, e.g., A to T substitution at the −43 nucleotides position counting upstream from the last nucleotide of the intron/splice acceptor in SEQ ID NO: 2, using the numbering of SEQ ID NO: 2.

SEQ ID NO: 3 is the DNA sequence depicting an exemplary CTE (constitutive transport element) of the present invention derived from Simian Mason-Pfizer D-type retrovirus (MPMV/6A).

SEQ ID NO: 4 is the DNA sequence depicting an exemplary WPRE (woodchuck hepatitis virus post-transcriptional response element) of the present invention derived from a source Woodchuck hepatitis virus with mutations (e.g., a base flip mutation between positions corresponding to A412 and T434 of SEQ ID NO: 4, using the numbering of SEQ ID NO: 4) to inactivate the potential start site for a cancerogenic X-protein and a compensating mutation to prevent secondary structure change.

SEQ ID NO: 5 is the DNA sequence depicting an exemplary internal ribosomal entry site (IRES) of the present invention derived from encephalomyocarditis virus (EMCV).

SEQ ID NO: 6 is the DNA sequence depicting an exemplary internal ribosomal entry site (IRES) of the present invention derived from Hepatitis C virus (HCV).

SEQ ID NO: 7 is the DNA sequence depicting an exemplary A-homopolymer of the present invention (i.e., an exemplary 50mer).

SEQ ID NO: 8 is the amino acid sequence of an exemplary Cre-recombinase of the present invention with C-terminal c-Myc NLS (nuclear localization signal).

SEQ ID NO: 9 is the amino acid sequence of an exemplary Streptococcus pyogenes Cas9 of the present invention with C-terminal tandem SV40 NLS (nuclear localization signal) and the HA epitope tag.

SEQ ID NO: 10 is the amino acid sequence of an exemplary FIp-recombinase of the present invention with C-terminal c-Myc NLS (nuclear localization signal).

SEQ ID NO: 11 is the amino acid sequence of an exemplary i53 polypeptide of the present invention, which is a genetically encoded 53BP1 (e.g., UniProtKB Accession Number: Q12888) inhibitor that suppresses non-homologous end-joining (NHEJ), so that homologous recombination (HR) alias homology-directed repair (HDR) is more efficient or is favored. 53BP1 is a positive regulator of NHEJ and a negative regulator of HR, thus inhibition of 53BP1 increases the efficiency of HR-mediated knock-in of a desired nucleic acid of interest. SEQ ID NO: 11 can be co-expressed on a separate plasmid or as P2A fusion to Cas9 (or any other DSB-inducing protein, independent if RNA- or amino acid-guided). SEQ ID NO: 11, as depicted herein, is the original unmodified i53 amino acid sequence, e.g., as reported by Canny et al., 2018 (Nat. Biotechnol. 2018 January; 36(1):95-102. doi: 10.1038/nbt.4021. Epub 2017 Nov. 27).

SEQ ID NO: 12 is the DNA sequence depicting an exemplary artificial construct of the present invention also designated as the loxP-WT_loxP-2272_synthetic-pA-rv_SV40-late-pA-mut-rv_rabbit-beta-globin-pA-mut-rv_rabbit-beta-globin-2nd-intron-SA-rv_loxP-WT-rv_rabbit-beta-globin-2nd-intron-SD-rv_loxP-2272-rv construct. For example, such construct can be used to produce a Cre-mediated irreversible KO of RNA-polymerase II (RNA-pol-II) driven gene. RNA-pol-II, because polyA are normally recognized canonically by RNA-pol-II driven transcription and terminating complex.

SEQ ID NO: 13 is the DNA sequence, depicting an exemplary intron-encoded secretory-NLuc of the present invention with synthetic SD (splice donor), SA (splice acceptor) of the present invention, a reporter (F3-sites-flanked-EF1a-Puro-2A-HSV-TK-cassette) and a flexed SA-triple-polyA signal. F3 sites are a mutant derivative of FRT sites, which are recognized by the FIp recombinase, both sites function in the same way and both are recognized by the same recombinase. However, F3 only recombines with F3 sites and WT FRT sites only with its WT sequence. This semi-orthogonality can be used in the Cre-inducible off-switch, using two semi-orthogonal loxP sites. F3 sites are flanking an inverted EF1a-promoter-driven puromycin n-acetyltransferase-P2A-thymidine-kinase expression constructs, terminated by the inverted polyA construct. Thus, the inverted loxP-sites flanked pA site having two functions, it functions first as a canonical polyA signal during the selection of the transgenic cells. After FIp-recombinase-mediated excision of the F3-flanked nucleic acid sequences, the inverted polyA remains within the intronic environment and functions as a Cre-inducible KO-switch for the host-gene (e.g., the gene, where the intron resides).

SEQ ID NO: 14 is the amino acid sequence of the intron-encoded secretory-NLuc as deducted from SEQ ID NO: 13.

SEQ ID NO: 15 is the DNA sequence depicting an exemplary loxP-WT fragment of SEQ ID NO: 12, i.e., a nucleic acid sequence, recognized by the Cre-recombinase.

SEQ ID NO: 16 is the DNA sequence depicting an exemplary loxP-2272 fragment of SEQ ID NO: 12, i.e., a nucleic acid sequence derived from loxP-WT sequence, recognized by the Cre-recombinase, which is semi-orthogonal (also called heterospecific) towards the WT sequence and Cre-recombinase, meaning that it only recombines with sites, which are identical to loxP-2272, but not with WT, wherein all are recognized by the same type of WT Cre-recombinase.

SEQ ID NO: 17 is the DNA sequence depicting an exemplary synthetic-pA-rv fragment of SEQ ID NO: 12, i.e., a synthetic polyA signal derived from the rabbit beta globin gene in its inverted direction (e.g., from a host-gene's point of view, e.g., Levitt et al., 1989; Genes Dev. 1989 July; 3(7):1019-25).

SEQ ID NO: 18 is the DNA sequence depicting an exemplary SV40-late-pA-mut-rv fragment of SEQ ID NO: 12, i.e., a mutant variant of the SV40 bidirectional polyA signal. The directions may be called “late” and “early” polyadenylation signal. It is placed in a way that the “late” signal is inverted from the host-gene's point of view. In the “early” SV40 pA direction, both AATAA motifs are mutated to disrupt the SV40 early pA signal. The reason is to have a Cre-mediated inversion of the “flexed” triple polyA signal, which shall have no polyA signal in the gene's sense direction when not “activated”/inverted.

SEQ ID NO: 19 is the DNA sequence depicting an exemplary rabbit-beta-globin-pA-mut-rv fragment of SEQ ID NO: 12, i.e., a polyA signal from rabbit beta globin gene in its inverted direction (from the host-gene's point view).

SEQ ID NO: 20 is the DNA sequence depicting an exemplary rabbit-beta-globin-2nd-intron-SA-rv fragment of SEQ ID NO: 12, i.e., the splice acceptor in its inverted (reverse complement) direction.

SEQ ID NO: 21 is the DNA sequence depicting an exemplary loxP-2272-rv fragment of SEQ ID NO: 12, i.e., a nucleic acid sequence derived from loxP-WT sequence in its inverted (reverse complement) direction, recognized by the Cre-recombinase, which is semi-orthogonal towards the WT sequence and Cre-recombinase, meaning that it only recombines with sites, which are identical to loxP-2272, but not with WT, wherein all are recognized by the same type of WT Cre-recombinase.

SEQ ID NO: 22 is the DNA sequence depicting an exemplary rabbit-beta-globin-2nd-intron-SD-rv fragment of SEQ ID NO: 12, i.e., a splice donor in its inverted (reverse complement) direction.

SEQ ID NO: 23 is the DNA sequence depicting an exemplary loxP-WT-rv fragment of SEQ ID NO: 12, i.e., a nucleic acid sequence, recognized by the Cre-recombinase in its inverted (reverse complement) direction.

SEQ ID NO: 24 is the DNA sequence depicting an exemplary reporter, F3-sites-flanked-EF1a-Puro-2A-HSV-TK-cassette. F3 sites are mutant derivatives of FRT sites, which are recognized by the FIp recombinase, both sites function in the same way and both are recognized by the same recombinase. However, F3 only recombines with F3 sites and WT FRT sites only with its WT sequence. This semi-orthogonality is used in the Cre-inducible off-switch using two semi-orthogonal loxP sites. F3 sites are flanking an inverted EF1a-promoter-driven puromycin n-acetyltransferase-P2A-thymidine-kinase expression construct, terminated by the also inverted polyA construct. Thus, the inverted loxP-sites flanked pA site has two functions, firstly, it functions as a canonical polyA signal during the selection of the transgenic cells. After FIp-recombinase-mediated excision of the F3-flanked nucleic acid sequences, the inverted polyA remains within the intronic environment and functions as a Cre-inducible KO-switch for the host-gene (e.g., a gene, where the intron resides).

SEQ ID NO: 25 is the DNA sequence depicting an exemplary CTE (constitutive transport element) with additional nucleotides derived from Simian-Mason-Pfizer D-type retrovirus (MPMV/6A).

SEQ ID NO: 26 is the DNA sequence depicting an exemplary chimeric fusion of crRNA and tracrRNA of Streptococcus pyogenes with mutations to prevent premature transcript termination and to improve sgRNA-folding and generic 20 nucleotides (e.g., any 20 nucleotides, e.g., n=a or g or c or t) spacer sequence shown as (N)₂₀. Sequence is shown with 3′-terminal 6×T, e.g., for RNA-polymerase III promoter driven transcript termination).

SEQ ID NO: 27 is the DNA sequence depicting an exemplary chimeric fusion of crRNA and tracrRNA of Streptococcus pyogenes with mutations to prevent premature transcript termination and to improve sgRNA-folding, without generic 20 nucleotides spacer sequence depicted in SEQ ID NO: 26. Sequence is shown with 3′-terminal 6×T, e.g., for RNA-polymerase III promoter driven transcript termination).

SEQ ID NO: 28 is the DNA sequence depicting an exemplary non-engineered sgRNA from Streptococcus pyogenes shown with 3′-terminal 6×T, e.g., for RNA-polymerase III promoter driven transcript termination, generic 20 nucleotides (e.g., any 20 nucleotides, e.g., n=a or g or c or t) spacer sequence shown as (N)₂₀; 4×T of the original scaffold leads to 80% premature termination with the typical used U6 RNA-polymerase III promoter.

SEQ ID NO: 29 is the DNA sequence depicting an exemplary NEAT1 spacer targeting the exon-of-interest.

SEQ ID NO: 30 is the DNA sequence depicting an exemplary NEAT1 primer 1.

SEQ ID NO: 31 is the DNA sequence depicting an exemplary NEAT1 primer 2.

SEQ ID NO: 32 is the DNA sequence depicting an exemplary reporter integrated KO-switch status primer 1.

SEQ ID NO: 33 is the DNA sequence depicting an exemplary reporter integrated KO-switch status primer 2.

SEQ ID NO: 34 is the amino acid sequence of Cas12c1, e.g., as reported by Yan et al., 2019 (Science. 2019 Jan. 4; 363(6422):88-91. doi: 10.1126/science.aav7271. Epub 2018 Dec. 6).

SEQ ID NO: 35 is the amino acid sequence of Cas12c2, e.g., as reported by Yan et al., 2019 (Science. 2019 Jan. 4; 363(6422):88-91. doi: 10.1126/science.aav7271. Epub 2018 Dec. 6).

SEQ ID NO: 36 is the amino acid sequence of OspCas12c derived from Oleiphilus sp. H10009, e.g., as reported by Yan et al., 2019 (Science. 2019 Jan. 4; 363(6422):88-91. doi: 10.1126/science.aav7271. Epub 2018 Dec. 6).

SEQ ID NO: 37 is the DNA sequence depicting an exemplary CTEv4 RNA export motif.

SEQ ID NO: 38 is the DNA sequence depicting an exemplary RNA stabilization motif, MmuMalat1 triple helix.

SEQ ID NO: 39 is the DNA sequence depicting an exemplary CTEv2 RNA export motif.

SEQ ID NO: 40 is the DNA sequence depicting an exemplary CAE-ml RNA export motif.

SEQ ID NO: 41 is the DNA sequence depicting an exemplary RTEm26-ml RNA export motif.

SEQ ID NO: 42 is the DNA sequence depicting an exemplary WPRE-m2 RNA export motif.

SEQ ID NO: 43 is the DNA sequence depicting an exemplary TAP-CTE-m1 RNA export motif.

SEQ ID NO: 44 is the RNA sequence depicting an exemplary CTE (constitutive transport element) of the present invention (which can be also referred to as “CTEv4” alias “CTE**” or “C**” herein).

SEQ ID NO: 45 is the DNA sequence depicting an exemplary RNA stabilization motif, Malat1 triple helix (which can also be referred to as “th” herein).

SEQ ID NO: 46 is the DNA sequence depicting an exemplary XAP1 plus self-complementary flanking sequences of the present invention.

SEQ ID NO: 47 is the DNA sequence depicting an exemplary xrRNA element (i.e., xrRNA1) of the present invention.

SEQ ID NO: 48 is the DNA sequence depicting an exemplary xrRNA element (i.e., xrRNA2) of the present invention.

SEQ ID NO: 49 is the DNA sequence depicting an exemplary xrRNA element (i.e., xrRNA containing xrRNA 1 and xrRNA2 with linker sequences) of the present invention.

SEQ ID NO: 50 is the DNA sequence depicting an exemplary 3′-HCV-UTR of the present invention (e.g., derived from Hepatitis C virus (HCV)).

SEQ ID NO: 51 is the amino acid sequence depicting an exemplary minimalGag-GCN4-PCP element/construct of the present invention.

SEQ ID NO: 52 is the amino acid sequence depicting an exemplary minimalGag2-GCN4-PCP element/construct of the present invention.

SEQ ID NO: 53 is the amino acid sequence depicting an exemplary dDBR1 element/construct of the present invention.

SEQ ID NO: 54 is the amino acid sequence depicting an exemplary dDBR1-FLAG element/construct of the present invention.

### BRIEF DESCRIPTION OF THE FIGURES

FIG. 1 shows a scheme of the current methods to monitor gene expression of coding and non-coding transcripts. FIG. 1a shows that protein-coding genes are normally expressed from an RNA polymerase II promoter carrying a 5′-cap (m7G) and are polyadenylated. FIG. 1b shows that classical N- or C-terminal fusion proteins can be used to determine subcellular localization. FIG. 1c shows that using a viral internal ribosome entry site (IRES), multi-cistronic mRNAs can be created such that an endogenous gene can be tagged by the insertion of an IRES-reporter downstream of the stop codon of the coding sequence (CDS) in the 3′-UTR. FIG. 1d shows that 2A peptides, derived from virus elements, enable the co-translational formation of independent proteins in one translation round via a ribosome skipping mechanism. FIG. 1e shows that intrabody fusions to fluorescent proteins allow the indirect subcellular tracking of a POI. FIG. 1f shows that the methods from b-c for coding genes are not applicable for non-coding RNAs since many of them are located in the nucleus where translation does not occur. Moreover, these methods are invasive as they heavily modify the RNA sequence and structure. FIG. 1g shows that the only established method to track RNA longitudinally and obtain subcellular resolution are aptamer-based two-component systems, where the first is a multi-dentate RNA-aptamer motif introduced into the DNA encoding the RNA of interest and a second part is an aptamer-binding-protein to fluorescent protein fusion. The latter is constitutively expressed from a safe-harbor locus (AAVS1 locus in human cells, Rosa26 in human and murine systems). This method necessitates modifications of the lncRNA with possibly adverse consequences regarding the stability and lifetime of the sequence.

FIG. 2 shows a scheme of gene transcription, transcript modification, export and how the endogenous process is modified by the intron-encoded transcript. FIG. 2A shows canonical gene expression of most protein-coding genes are driven by an RNA-polymerase II promoter, and 95% of them contain introns that are excised co-/post-transcriptionally, leaving the remaining exons ligated scarlessly. This mechanism is called RNA-splicing and is one of the major steps beside 5′-capping (addition of a 7-methylguanylate cap to the 5′-end of the de-novo transcribed RNA) and 3′-polyadenylation (addition of poly(A) tail to the RNA) resulting in a mature mRNA. Some exons are alternatively spliced, resulting in isoforms with and without this exon. A complex called exon-junction-complex (EJC) will mark the position ˜50 nt upstream of an exon-exon-junction after splicing. Afterwards, a variety of proteins bind to the 5′-cap and the poly(A)-tail, stimulating the nuclear export of the mature mRNA. The excised intron is degraded after the 2′-5′-phosphodiester bonds of the circular intron is de-branched by DBR1. Afterwards, the exported mRNA, the 5′-cap-binding and poly(A)-binding proteins initiate translation of the CDS by recruiting the ribosomal subunits. The 5′- and 3′-untranslated region (upstream of the start codon ATG and downstream of the stop codon TAA/TGA/TAG) are called 5′-UTR and 3′-UTR. FIG. 2B shows a scheme of gene transcription, transcript modification and export, equipped with an intron-encoded protein translation system. The internal ribosome entry site enables 5′-cap-independent translation of an effector protein that can encode proteinogenic reporters and/or sensors. The RNA nuclear export signal/motif enables 5′-cap-, polyA-, and EJC-independent export of the intronic RNA that is degraded otherwise. FIG. 2C shows a scheme of gene transcription, transcript modification and export, equipped with an intron-encoded RNA-effector, more specifically an RNA-sensor or -reporter system. Shown here is an exemplary sensor-effector that encodes an aptamer that fluoresces (reporter) upon a specific metabolite (sensor) using an otherwise non-fluorogenic fluorophore. The RNA nuclear export signal/motif enables the export of the intronic RNA that is degraded otherwise inside the nucleus. FIG. 2D shows a scheme of gene transcription, transcript modification and export, equipped with an intron-encoded RNA-barcode, that is additionally exported via the exosomal secretion pathway using motifs (exosomal loading motifs) facilitating exosomal packaging. The RNA nuclear export signal/motif enables the export of the intronic RNA that is degraded otherwise inside the nucleus and thereby enables the packaging of the barcode into exosomes using the exosomal ZIP-code. Readout of the barcodes is performed using RT followed by NGS or other single-cell sequencing formats that is also compatible to sequence single exosomal vesicles. FIG. 2E is a modification of FIG. 2d, where the barcode is embedded within an artificial microRNA that contains a microRNA-specific exosomal targeting motif that enables the secretion of microRNAs via the exosomal pathway. FIG. 2F is a combination of FIGS. 2b and 2d. It combines the proteinogenic coding capability with the RNA-barcoding system. The encoded protein is a DNA-modifying enzyme that preferentially modifies the DNA via base-editing and thereby the barcode is evolving. Depending on the base-editing frequency, the barcodes act as a unique cellular identifier (slow mutation rate) or as a timestamp (fast mutation rate). Similar to FIG. 2d, the secreted continuously evolving barcodes are readout via RT followed by NGS or other sequencing technologies such as single-cell transcriptome sequencing technologies. FIG. 2G shows exemplary types of intron-specific information that can be encoded either at the RNA or protein level to serve as a reporter, sensor, or actuator. FIG. 2H tabulates the advantages of the method for non-invasive monitoring of gene expression disclosed herein.

FIG. 3 shows the introduction of elements of endogenous or synthetic introns into exonic sequences. This schematic diagram describes how intronic sequences can be embedded into exonic sequences such that the transcriptional activity of a gene of interest can be read out without changing its mature mRNA or lncRNA. To test the feasibility of this approach, the inventors expressed transiently from a plasmid an mRNA encoding the CDS for mNeonGreen. Additionally, within the CDS, the inventors embedded a synthetic intron including an intron-encoded CDS for a secretory NanoLuc luciferase (NLuc). The inventors combined different elements from RNA viruses known to mediate nuclear export of the viral genome and intron-encoded cap-independent translation in a non-canonical way to generate a functional eukaryotic intron-encoded protein, which is independent of the co-transcribed mRNA, but still reports the transcriptional activity of its host promoter. Elements stimulating nuclear export: a) CTE: constitutive transport element from Mason-Pfizer monkey virus (MPMV), b) WPRE: Woodchuck Hepatitis virus post-transcriptional regulatory element (WPRE), poly(A): homopolymeric tracts of adenine bases. Elements enabling cap-independent translation: internal ribosome entry sites (IRES) from a) Hepatitis C virus (HCV) or from b) encephalomyocarditis virus (EMCV).

FIG. 4 shows the engineering of an eukaryotic intron-encoded, extranuclear cap-independent protein-coding transcript. FIG. 4a shows that to assess the ability to encode proteins within an intronic sequence, the inventors used a secreted Nanoluc luciferase (NLuc) as intron-encoded protein and inserted the intronic sequence within an exonic mRNA encoding for a nuclear-localized mNeonGreen driven by a constitutive hybrid mammalian CAG promoter. To enable the translation of an intron-encoded CDS, the intron has first to be exported to the nucleus after its excision, while escaping the native degradation pathway and secondly, a cap-independent translation has to be initiated. The inventors combined different elements from RNA viruses known to mediate nuclear export of the viral genome and intron-encoded cap-independent translation in a non-canonical way to generate a functional eukaryotic intron-encoded protein, which is independent of the co-transcribed mRNA, but still reports the transcription activity of its host promoter. Elements stimulating nuclear export: CTE: constitutive transport element from Mason-Pfizer monkey virus (MPMV), WPRE: Woodchuck Hepatitis virus post-transcriptional regulatory element (WPRE), poly(A): homopolymeric tracts of adenine bases. Elements enabling cap-independent translation: internal ribosome entry sites (IRES) from Hepatitis C virus (HCV) or encephalomyocarditis virus (EMCV). FIG. 4b shows the different elements that were combined or put in tandem to optimize the nuclear export and translation efficiency of the intronic RNA containing HCV-IRES; read-out via the intron-encoded secreted NLuc. The supernatant of the samples were collected at the indicated time points post-transfection. FIG. 4c shows the different elements that were combined or put in tandem to optimize the nuclear export and translation efficiency of the intronic RNA containing EMCV-IRES; read-out via the intron-encoded secreted NLuc. The supernatant of the samples was collected at the indicated time points post-transfection. FIG. 4d shows the representative epifluorescence images cells expressing the exon-encoded mNeonGreen-NLS transfected with the indicated constructs. FIG. 4e shows the optimization of the nuclear export motifs and stabilizing motifs using a dual-luciferase system. The intron-encoded NanoLuc within the intron is inserted into the firefly luciferase CDS. After transfection, the intron is spliced out and exonic FLuc, as well as intronic NLuc, are expressed separately. Two days post-transfection dual-luciferase assay is performed for evaluation of the results. PEST degradation signal is fused to both, NanoLuc and firefly luciferase, to destabilize the luciferases for a more dynamic signal response. Malat1 triple helix was also tested, which stabilizes the 3′-end of a linear RNA. CTEv4, e.g., SEQ ID NO: 37 is a variant of CTE without a potential detrimental cryptic splice donor. MmuMalat1 triple helix (e.g., SEQ ID NO: 38) is an RNA-stabilizing motif that is derived from the lncRNA Malat1 that protects the 3′-end from degradation. FIG. 4f shows the results from the optimization of the nuclear export motifs and stabilizing motifs from FIG. 4e. FLuc (exonic signal) indicates the integrity of the exon and thus the RNA-splicing itself. NLuc (intronic signal) indicates the nuclear export and translation efficiency of the otherwise degraded intron. Construct IDs 3 and 4 were 20-30-fold better compared to the control construct without nuclear export or stabilization motifs.

FIG. 5 shows the application of the intron-encoded extranuclear transcript for non-invasive expression of a translocon-dependent multipass-transmembrane protein. FIG. 5a shows a prototype intron-encoded multipass transmembrane protein, sodium iodine symporter (NIS alias SLC5A5) that was used, which was transfected into HEK293T cells. Its expression was quantified via the accumulation of the -emitter ¹³¹I⁻. FIG. 5b shows that after the indicated incubation time with sodium iodide (¹³¹I isotope), the accumulated ¹³¹I⁻ in the lysed samples was measured via a γ-scintillator. FIG. 5c shows the epifluorescence microscopy images of exonic mNeonGreen-NLS, expressing the indicated intron-encoded NIS or secretory NLuc. FIG. 5d shows that the intron-encoded NIS could be integrated within the IL2 gene, which is transcriptionally induced in activated (CAR)-T-cells enabling longitudinal non-invasive monitoring of activated (CAR)-T-cells using positron emission tomography (PET) and single-photon emission computed tomography (SPECT) via the accumulation of radioactive I⁻ isotopes.

FIG. 6 shows the design of the Cre-inducible KO-switch based on the intron-encoded extranuclear transcript system. FIG. 6a shows the used plasmid-expressed mNeonGreen as our surrogate gene to test the KO-switch. Beside the intron-encoded reporter system, the inventors additionally integrated an inverted EF1a promoter-driven selection cassette encoding for the puromycin N-acetyltransferase (PuroR) and the viral thymidine kinase (HSV-Tk), co-expressed via a P2A ribosome skipping peptide. The selection cassette enables positive selection after nuclease-mediated KI of the intron-encoded transcript into the gene of interest. FIG. 6b shows that afterwards, the cassette is removed by FIp recombinases. Only the promoter-CDS moiety is flanked by mutant variant F3 of FRT-sites and thus is excised via transfection of a plasmid encoding for FIp recombinases. The inverted composite part comprising the splice donor (SD), splice acceptor (SA), and the triple poly(A) (pA) signal, is thus not removed. FIG. 6c shows that the SA-pA part is “FLExed”, meaning two different semi-orthogonal loxP sites (lox2272 and loxP WT sites are both not compatible, but are both recognized by the same Cre recombinase) are flanking the SA-pA part in a way, that, upon Cre recombinase expression, this part will be irreversible flipped in its non-inverted direction. The SD part is positioned in a way that it will be removed after Cre-mediated SA-pA inversion. Since Cre recombinase leads to the restoration of the SA-pA in the sense direction of any tagged gene, it will lead inevitably to the KO of the gene by premature polyadenylation by the restored poly(A) signal. The SA ensures that the poly(A) signal is not accidentally skipped, since some introns splice within seconds, which might lead to an ineffective premature transcript termination. The SA from the switch prevents the usage of the downstream SA. The SA_poly(A) transcript is redefined as an exonic sequence after Cre-mediated inversion into the genes' sense direction and thus ensures the premature transcript termination. The effect of FIp or Cre recombinases on the plasmid-based test-constructs expressing exonic mNeonGreen and intron secretory NLuc with the Cre-inducible KO-switch are readout via the bioluminescence signal of NLuc, as shown as in FIG. 6c in the supernatant and as in FIG. 6e, via epifluorescence microscopy of the nuclear-localized mNeonGreen.

FIG. 7 shows that the intron-encoded extranuclear transcript system enables non-invasive and longitudinal monitoring of long non-coding RNAs (lncRNAs) with an integrated Cre-inducible KO-system. FIG. 7a shows that the inventors knocked the reporter construct into the lncRNA NEAT1_v1, which is also a part of the long isoform NEAT1_v2. FIG. 7b shows the FIp-mediated excision of the EF1a-PuroR-P2A-HSV-Tk and FIG. 7c shows the Cre-mediated KO of NEAT1. FIG. 7d shows the representative smFISH images of probes binding to the region of NEAT1_v1/v2 and NEAT1_v2 of unmodified 293T cells, the reporter without (NEAT1:SP-NLuc) and with Cre-activated off-switch. FIG. 7e shows the relative luminescence of the supernatant 48 h post-seeding of indicated cells (unmodified HEK293T, NEAT1:SP-NLuc, NEAT1:SP-NLuc+Cre, technical duplicates shown as data points). FIG. 7f shows a quantification of paraspeckle containing cells (using Quasar670 signal of NEAT1_v1/v2). **** denoting p-values smaller than 0.0001 (binomial test, two-tailed).

FIG. 8 shows a nested dual-luciferase system for optimizing nuclear export, RNA stability and 5′-cap-independent translation of “INSPECT”. The term “INPECT” as used in the context of the present invention and as used herein means intron-encoded scarless programmable extranuclear cistronic transcript, a minimally-invasive transcriptional reporter embedded within an intron of a gene of interest. INSPECT can be applied as the first method for monitoring gene transcription without altering the target of interest at either the RNA or protein level. FIGS. 8a and 8b show that the synthetic intron was nested within a FLuc:PEST coding sequence on a plasmid system driven by the mouse Pgk1 promoter. In addition, an intron-encoded translational unit, IRES:NLuc-PEST was inserted into the artificial intron, composed of two highly efficient splice sites (splice donor and splice acceptor, SD & SA) for insertion of further genetic elements for nuclear export or RNA stability at the 5′- and 3′-end. The system was tested by transient transfection of HEK293T cells, followed by a dual luciferase assay after 48 h expression. The effect of different genetic elements on the ability to express proteins from an intron (combined effect of nuclear export of the intron and translational efficiency of the intron-encoded protein) was validated by the NLuc signal, while detection of the FLuc signal indicated correct splicing of the exonic sequence. FIG. 8c shows that the system features a Cre-recombinase-inducible KO-switch by encoding an inverted triple poly(A)-signal flanked by two heterospecific loxP-pairs (heterologous means that loxP only recombines with loxP and lox2272 only with lox2272, but both are recognized by the same recombinase). Upon transfection of the Cre recombinase, the poly(A) sites, together with an upstream splice acceptor, is inverted into sense and leads to two independent KO-ensuring events: 1) the activation of the BP (branch point) and the downstream SA induces mis-splicing, which will destroy the native splicing of any gene and 2) premature transcriptional termination by the active poly(A) sites. FIGS. 8d-f show the results of the dual-luciferase assay, shown in FIG. 8a, to test the ability to enhance the expression of the intron-encoded NLuc:PEST without detrimental effects on the exonic expression (FLuc:PEST). Different variants of nuclear export and RNA-stabilization elements were tested either at the 5′-site or 3′-site (relative to IRES:NLuc-PEST) and also tandem repeats thereof were used (number of repeats indexed as subscript). CTE: constitutive transport element from Mason-Pfizer monkey virus, CTE*: variant of CTE, CTE**: another variant of CTE, RTE: m26 mutant of an RNA transport element with homology to rodent intracisternal A-particles, triplex: triple helix forming RNA from mouse Malat1 lncRNA for 3′-end stabilization. FIG. 8g shows the version containing 5′-2×CTE and 3′-2×CTE**, which were compared in the context of different IRES from either encephalomyocarditis virus (ECMV) or from the human gene vascular endothelial growth factor and type 1 collagen-inducible protein (VCIP). Cre: indicates the co-transfection of a plasmid expressing Cre-recombinase, which recognizes the heterospecific loxP and lox2272 to activate the KO switch (see FIG. 8c). The bars represent the mean of three biological replicates with the error bar representing the standard deviation.

FIG. 9 shows the homozygous integration of the “INSPECT” reporter system, which allows monitoring of NEAT1 gene expression without interfering with paraspeckle formation. FIGS. 9a and 9b show the v1 version of the reporter system (see FIG. 8) equipped with a secreted NLuc (SecNLuc), which was inserted via CRISPR-Cas9 into different sites of the lncRNA NEAT1. The lncRNA NEAT1 is transcribed into a short and a long RNA isoform, where the latter one is essential for the formation of ‘paraspeckles’ in complex with several RNA-binding proteins. Insertion site 1 (IS1) is present in both isoforms, IS7 and IS8 report long isoform expressions exclusively. FIG. 9c shows that the system integrated into NEAT1 also features a Cre-recombinase-inducible KO-switch (see FIG. 8d for details). FIG. 9d shows that for each insertion site, a representative image of the DAPI- and probe-channel (depicting NEAT1 smFISH signals) are depicted. Bottom pictures of each sub-panel illustrate which signals of the probe channel were identified as nucleus (circles) and paraspeckles (+) and were used to count the respective nuclei and paraspeckles automatically. Clone v0 originates from preliminary reporter generation. If not otherwise indicated, v1 was used. FIG. 9e shows the RLUs of secNLuc in the supernatant after 72 hours of transfection with plasmids for CRISPRi of NEAT1 via plasmids encoding a dCas9:transcriptional-repressor fusion chimera targeted with three sgRNAs against the NEAT1 promoter (24 hours before measurement, medium was changed to reset the signal). FIG. 9f shows the % of cells containing paraspeckles for different insertion sites (see FIG. 9d for representative images), IS1* containing the prototype version (v0) was omitted from analysis since the speckles were morphologically distinct compared to wild type cells (n indicates the number of analyzed nuclei). IS1*+Cre were analyzed to show the efficiency of the KO via Cre-recombinase.

FIG. 10 shows that the “INSPECT” reporter enables modular read-out of coding genes using protein and RNA reporters. FIGS. 10a-c show that the TCR signaling can be artificially induced with the tripartite mixture of phytohaemagglutinin (PHA, 1 ng ml⁻¹), phorbol 12-myristate 13-acetate (PMA, 1 μg ml⁻¹), and the Ca²⁺ ionophore (Br)-A23187 (0.1 μM). The subsequent massive induction of IL2 can be read out via INSPECT v1, equipped with secNLuc or the sodium iodide symporter (NIS) knocked-in into exon 3 of the NFAT controlled IL2 locus in Jurkat E6.1 cells. FIG. 10d shows quantification of secreted IL2 by sandwich ELISA, bioluminescence in the supernatant (NLuc), or measured radioactive decay of the radioisotope I-131⁻ within the cells (NIS) 16 hours after T cell activation. The dashed line indicates the baseline A450 level of ELISA of non-activated Jurkat E6.1 cells. Shown are individual data points for n=3 independent clones for each reporter modality.

FIG. 11 shows further optimization of nuclear export, RNA stability and 5′-cap-independent translation of the intron-encoded reporter system. FIGS. 11a-11c show that the synthetic intron was nested within a sfGFP coding sequence (green fluorescence) on a plasmid system driven by the strong mammalian CAG promoter. In addition, an intron-encoded translational unit, IRES:mScarlet-I (red fluorescence) was inserted into the artificial intron already equipped with the v1 elements from before (see FIGS. 8f and 8g) and offers the opportunity to insert further genetic elements for nuclear export or RNA stability at the 5′- and 3′-end to enhance the v1 system even more. The system was tested by transient transfection of HEK293T cells, followed by FACS analysis 48 h post-transfection. The effect of different genetic elements on the ability to express proteins from an intron (cumulative effect of nuclear export of the intron and translational efficiency of the intron-encoded protein) was validated by mScarlet-I fluorescence (readout at 586 nm), while detection of the sfGFP signal indicated correct splicing of the exonic sequence (readout at 530 nm). FIG. 11d shows the results of FACS analysis readout at 530 nm (sfGFP, exonic signal, left) and 586 nm (mScarlet-I, intronic signal, right). Orange: v1 containing 5′-CTE and 3′-CTE** tandem insertion; v2.1: contains in addition to v1 additional 5′-xrRNA and 3′-XAP1; v2.2: contains in addition to v1 additional 5′-xrRNA and 3′-HCV-UTR.

FIG. 12 shows the extracellular export of “INSPECT” introns instead/in addition to the intron-encoded reporter, which enables longitudinal RNA-based analysis of gene expression. FIG. 12a is a schematic overview of the proof-of-concept constructs used in this experiment to show that the cytosolic intron can be equipped with additional RNA motifs, such as the PP7 RNA-aptamer, to be readily exported from the cytosol to the extracellular space by engineered gag chimeras (black ball-like structures) that are capable of binding the PP7 motifs via the binding protein PCP (PP7 coat protein). A gag-PCP export system was engineered and validated for exporting PP7-tagged “INSPECT” cytosolic introns to track the gene expression of the host gene. Two reporters were created, one with a constitutive promoter (Pgk1) and another with a doxycycline-inducible promoter (TRE3G). The constitutive promoter drives the expression of the red fluorescent protein mScarlet-I, while the inducible promoter drives the expression of a green fluorescent protein msfGFP. Both constructs contain “INSPECT” with a unique nucleotide barcode (probe sequence 1 and probe sequence 2) respectively within the intron to allow RNA-based analysis via RNA-sequencing or RT-qPCR quantification. FIG. 12b shows 24 h post-transfection with the indicated constructs from FIG. 12a, with a plasmid encoding the Tet-On 3G transactivator to enable doxycycline-inducible gene expression of the TRE3G promoter. Cells were induced with the indicated doxycycline concentrations. 48 h post-transfection, cells were quantified for red and green fluorescence (left chart indicating the average fluorescence in the respective fluorescence channels). In addition, the supernatant of the indicated conditions was used for RNA extraction and was analyzed subsequently via RT-qPCR to quantify the respective “INSPECT” species via the unique probe sites. Results of RT-qPCR of the indicated species are shown as Ct and ΔCt (n=3, biological replicates).

FIG. 13 shows the RT-qPCR results, shown as Ct and ΔCt of and improved miniature gag (minigag) chimeras, which enables less unspecific export of untagged RNA species, while maintaining the export efficiency of PP7-tagged RNA species. RNA was purified from HEK-293T cells' supernatant 48 hours post-transfection with the indicated VLP-forming plasmids co-transfected with a reporter plasmid with their corresponding 3′-UTR tagged with PP7 or psi (from HIV-1) (thick-lined circles). An untagged version was always co-transfected (thin-lined circles) to measure the unspecific secretion mediated by different VLP systems.

FIG. 14 shows the homozygous integration of the “INSPECT” reporter system into the IL2 locus, which allows monitoring of activated T cells without impairing endogenous gene expression. FIG. 14a shows the CRISPR/Cas9-mediated knock in of the INSPECT_{V1-NLuc} reporter into exon 3 of the NFAT controlled IL2 locus of Jurkat E6.1 cells. The synthetic intron is flanked by splice sites following the splice consensus. The reporter system comprises the tandem CTE elements for nuclear export, EMCV IRES for initiation of translation. A sensitive read out is enabled by secretion of a Nanoluc reporter protein after T-cell activation. FIG. 14b shows that IL-2 sandwich ELISA as well as NanoLuc signal from supernatant confirm IL2 expression 16 hours after T cell activation. IL2 expression in Jurkat E6.1 was induced with 1 ng/ml PMA, 1 μg/ml PHA and 0.1 μM calcium ionophore (Br)-A23187. FIG. 14c shows that the synthetic intronic sequence can also be utilized as RNA reporter providing a reporter sequence/sequence tag. The RNA transcript is secreted via gag virus-like particles (VLPs) derived from the lentivirus HIV-1. The gag polyprotein acts as a structural unit and is fused to the PP7 bacteriophage coat protein (PCP). After transcription, splicing and nuclear export of the synthetic intron, the gag-PCP fusion protein recognizes the PP7-tagged “INSPECT”, assembles to VLPs and buds off the cellular membrane, while effectively secreting the intronic RNA. FIG. 14d shows transient expression of a constitutive (mScarlet-I) and an inducible (msfGFP) surrogate gene. FIG. 14e shows that after splicing, the intronic RNA is secreted via VLPs and can be detected by RT-qPCR. Induction with doxycycline took place 12-16 h post-transfection. Fluorescence measurements and RNA isolation were carried out 48 h post-transfection. Average intensity of msfGFP and mScarlet fluorescence was measured via epifluorescence microscopy and matched with a corresponding RT-qPCR plot. Average intensity values were corrected with an untransfected control. Dotted lines indicate a no-RT threshold for each probe.

FIG. 15 shows how lariat debranching enzyme (DBR1) was able to mediate nuclear-cytosolic export of an intron containing no RNA nuclear export elements (NES) such as CTEs (condition labeled as “w/o RNA NES”). Catalytically dead DBR1 (dDBR1) mutant of DBR1 was created by introducing the H85A mutation in the catalytic domain of human WT DBR1. Co-transfection of the FLuc-NLuc test-construct with 5′- and 3′-RNA nuclear export elements (from FIG. 8b) was used as a positive control for effective nuclear-cytosolic export of intronic RNA (first pair of bars), while the control construct does not contain any RNA NES (w/o RNA NES, 2nd pair of bars). The model behind these data is that nuclear export mediated by dDBR1 (which has been reported to shuffle between nucleus and cytosol) is competing with the debranching activity and thus the degradation of introns mediated by the endogenous expressed active DBR1. Consequently, knock-down of endogenous DBR1 should increase the efficiency of the dDBR1-mediated nuclear-cytosolic shuffling of introns since degradation of introns is in generally diminished. Thus, dDBR1 was co-expressed with a control construct without RNA NES, in the presence and absence of additional microRNAs (miRs) targeting the endogenous enzymatically active DBR1 via its respective 3′-UTRs. Note that the heterologously expressed dDBR1 is not a target of the miRs, because it has a different non-native 3′-UTR. As expected, co-expression with miRs further increased the nuclear export activity of dDBR1 (bars in groups 4, 5, 6, and 7).

FIG. 16 shows a tabulation of an updated overview of existing genetically encoded approaches to monitor gene expression compared to INSPECT (FIG. 2). Fusion protein: A direct fusion (here C-terminal) of a reporter protein (CDS2) resulting in a fusion protein to the native sequences (CDS1). IRES: Internal ribosome entry sites mediates cap-independent translation of the 3′-cistron proportional to CDS 1 expression, but modifies the 3′-UTR of the endogenous mRNA. 2A: For stoichiometric translation of CDS 1 and CDS2, 2A sequences use a ribosome stalling mechanism, leaving scars on the host protein. A subset of nanobodies can be expressed in cells fused to fluorescent proteins (XFPs) to visualize target proteins. RNA aptamer: Insertion of MS2/PP7 RNA aptamers into the UTR of an mRNA or a non-coding RNA enables visualization via an aptamer-binding protein (ABP)-XFP fusions. Ents (Endogenous transcription-gated switch): The tripartite system is composed of a sgRNA flanked by tRNAs, integrated into the 3′-UTR of a gene, which is released by endogenous RNAse Z/P, resulting in a poly(A)-deficient host transcript, a free poly(A)-tail and a free sgRNA that in turn induces the expression of a separate integrated reporter system via a dCas9 transactivator system, which is also integrated into the genome. The host mRNA lacking the poly(A) tail then should be exported to the cytosolic environment. INSPECT: the intron encoded cistronic transcript is spliced, stabilized, exported from the nucleus into the cytosol for cap-independent translation or, alternatively, secreted from the cell as an RNA-barcode reporter.

### DETAILED DESCRIPTION OF THE INVENTION

The following detailed description refers to the accompanying examples and figures that show, by way of illustration, specific details and embodiments, in which the invention may be practiced. These embodiments are described in sufficient detail to enable those skilled in the art to practice the invention. Other embodiments may be utilized such that structural, logical, and eclectic changes may be made without departing from the scope of the invention. Various aspects of the present invention described herein are not necessarily mutually exclusive, as aspects of the present invention can be combined with one or more other aspects to form new embodiments of the present invention.

Unless otherwise specified, the terms used herein have their common general meaning as known in the art.

As described herein, references are made to UniProtKB Accession Numbers (http://www.uniprot.org/, e.g., as available in UniProtKB, UniProt release 2019_07, published on Jul. 31, 2019).

As described herein references are further made to GenBank Accession Numbers, GenBank Release 232, Jun. 15, 2019 (https://www.ncbi.nlm.nih.gov/genbank/release/).

As described herein references are further made to RefSeq Accession Numbers, RefSeq Release 96, Sep. 16, 2019 (https://www.ncbi.nlm.nih.gov/refseq/).

The relatedness between two amino acid sequences or between two nucleotide sequences may be described by the parameter “sequence identity” (or “% identity”). For purposes of the present invention, the sequence identity between two amino acid sequences may be determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment).

For purposes of the present invention, the sequence identity between two deoxyribonucleotide sequences may be determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labelled “longest identity” (obtained using the -nobrief option) is used as the percent identity and is calculated as follows:

(Identical Deoxyribonucleotides×100)/(Length of Alignment−Total Number of Gaps in Alignment).

The principles described below can be used for any protein or nucleic acid sequence described herein. For example, the sequence having SEQ ID NO: 4 can be used to determine the corresponding residue in another nucleic acid sequence or variant thereof. The sequence of another nucleic acid is aligned with the sequence having SEQ ID NO: 4, and based on the alignment, the residue position number corresponding to any residue in the SEQ ID NO: 4, is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of a corresponding residue in another sequence can be determined by an alignment of multiple sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 51 1-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

In describing the variants of the present invention, the nomenclature described below is adapted for ease of reference. The accepted IUPAC single letter or three letter amino acid abbreviation is employed. Substitutions. For an amino acid substitution, the following nomenclature is used: Original amino acid, position, substituted amino acid. Accordingly, the substitution of threonine at position 226 with alanine is designated as “Thr226Ala” or “T226A”.

Described herein is an innovative method for minimally invasive insertion, transcription and detection of a nucleic acid construct that is simultaneously expressed with an endogenous gene of interest. Both non-coding and coding RNAs can be encoded by the heterologous nucleic acid sequence or cargo, and will be transported out of the nucleus after transcription. Tagged coding and non-coding RNAs can be detected with this method, while coding RNAs may be detected as translated protein that may be tagged. Further the transcribed and later cytosolic coding or non-coding RNA may fulfil different tasks within the cell. Different scenarios are possible, like the silencing of an endogenous gene transcript, the enhancing of endogenous transcript or simply the reporting of the endogenous gene transcript at a given time point. Importantly only the simultaneously expressed endogens gene of interest is silenced, enhanced or reported in this context. Said method further includes that the integrated nucleic acid construct or cassette can be reused in a sense that the living cell will express the integrated heterologous nucleic acid sequence or cargo whenever the endogenous gene is expressed. This gives a time resolved picture of the gene expression in a living cell. This method enables for example the direct genetically induced treatment of pathologic events occurring in a living cell or tissue.

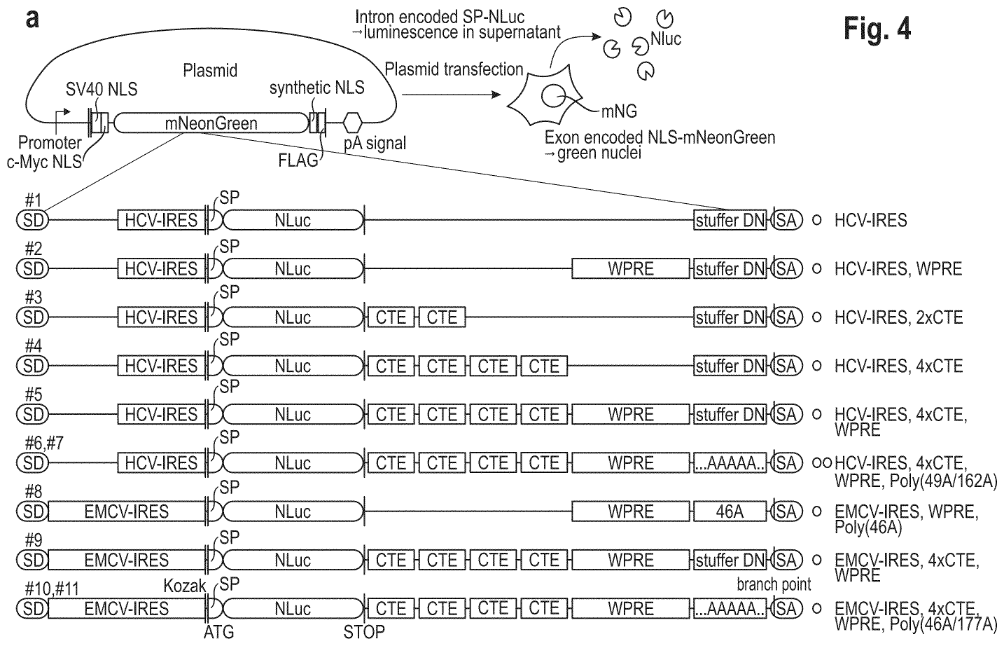

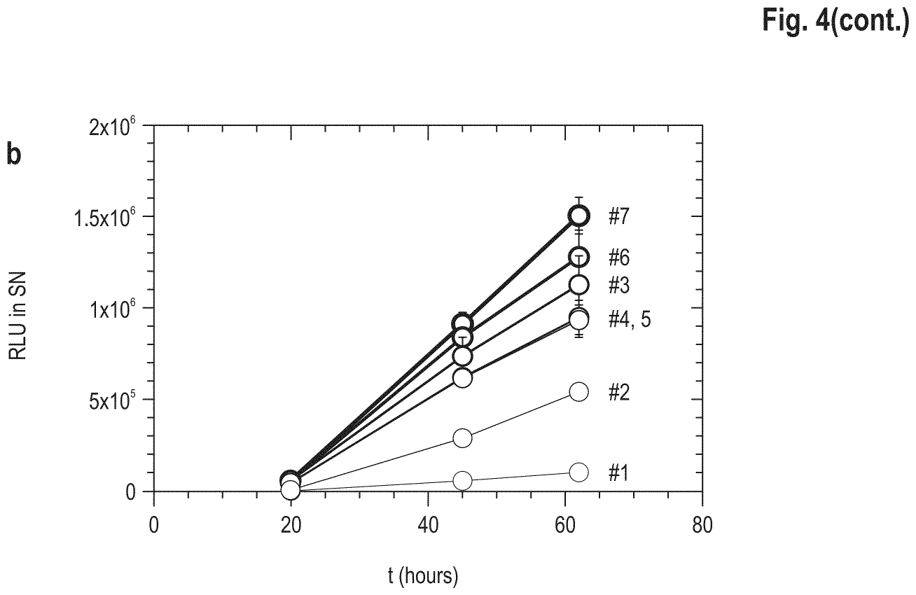

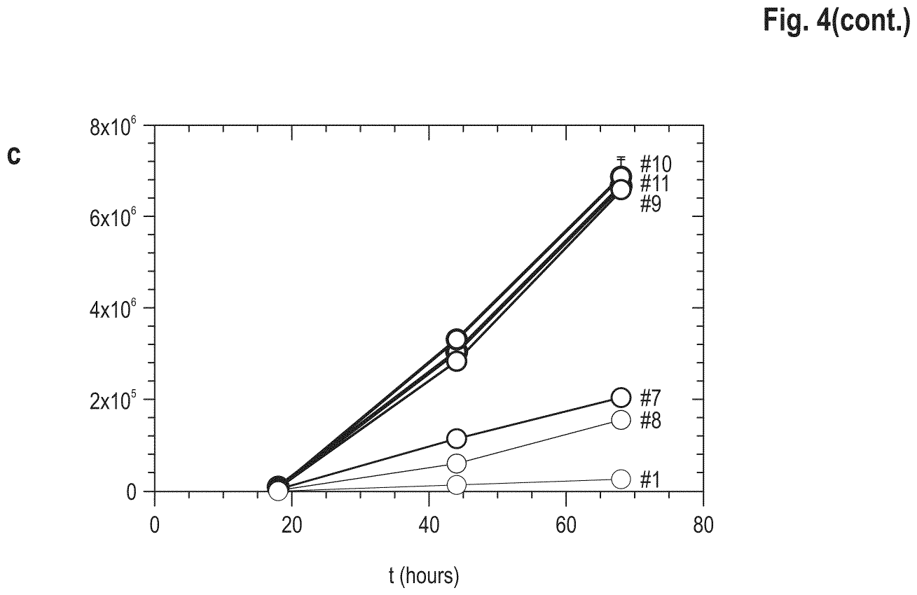

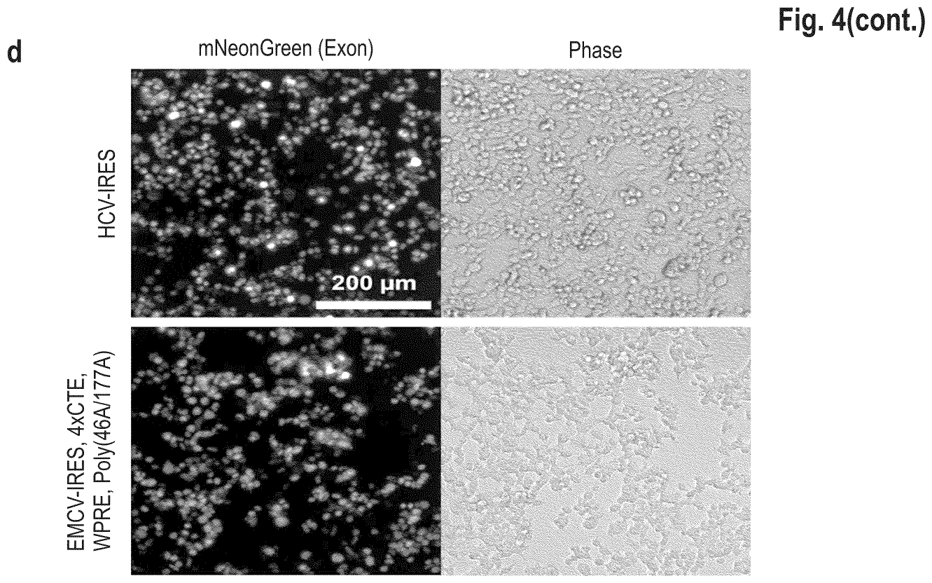

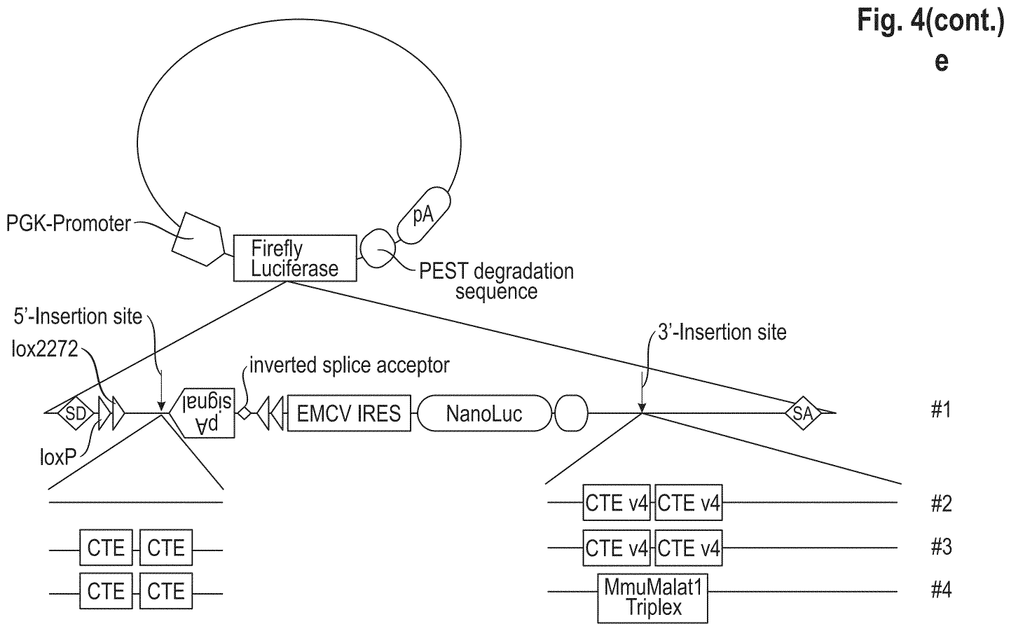

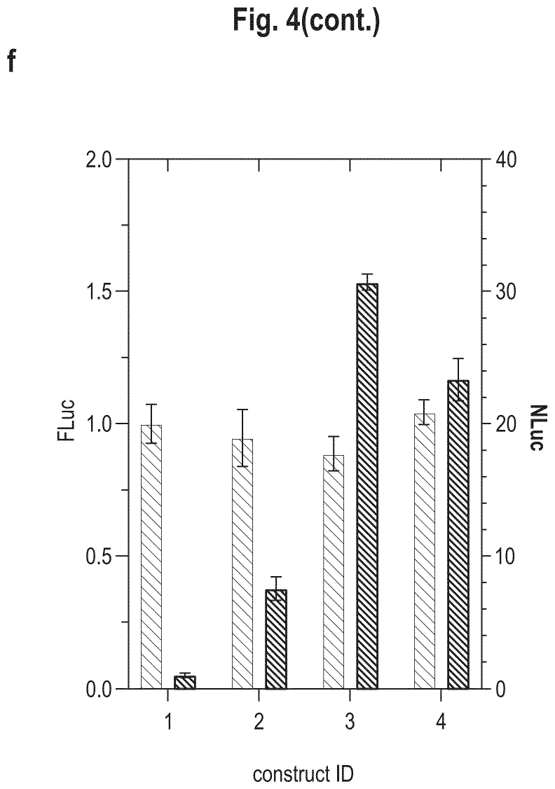

As an example of an intron-encoded protein the inventors used NanoLuc luciferase (NLuc) with an N-terminal secretion peptide (SP) from Gaussia princeps luciferase. The inventors permuted and combined different elements enabling cap-independent translation and cap- and poly(A) independent nuclear export elements and tested it transiently in HEK293T cells (FIG. 4a). The highest signal was measured with all structural components (WPRE, CTE pair downstream of HCV-IRES_SP-NLuc) combined (FIG. 4b). All constructs tested showed a similar expression of the exonic mNeonGreen, indicating the non-invasiveness of those reprogrammed introns (FIG. 4d).

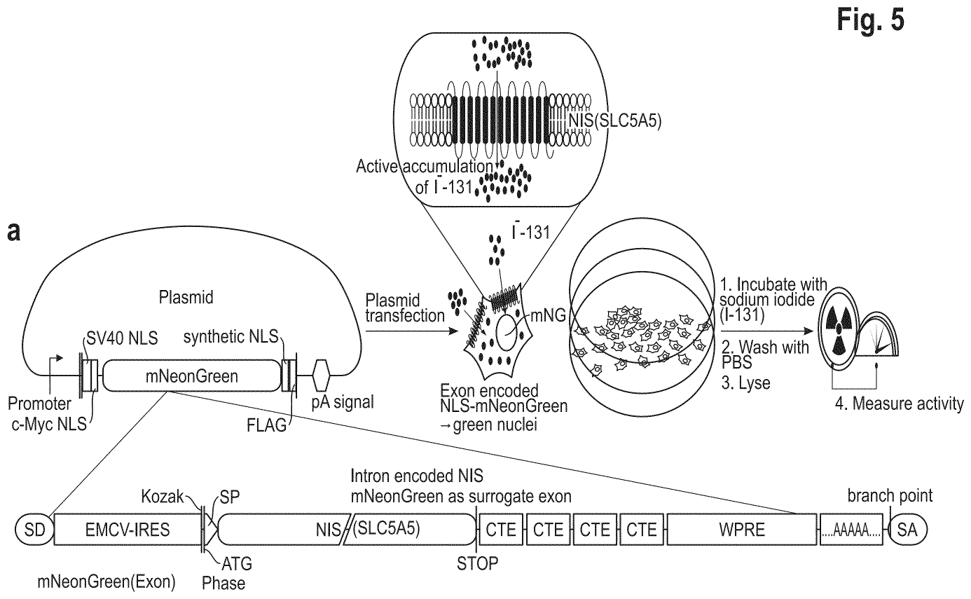

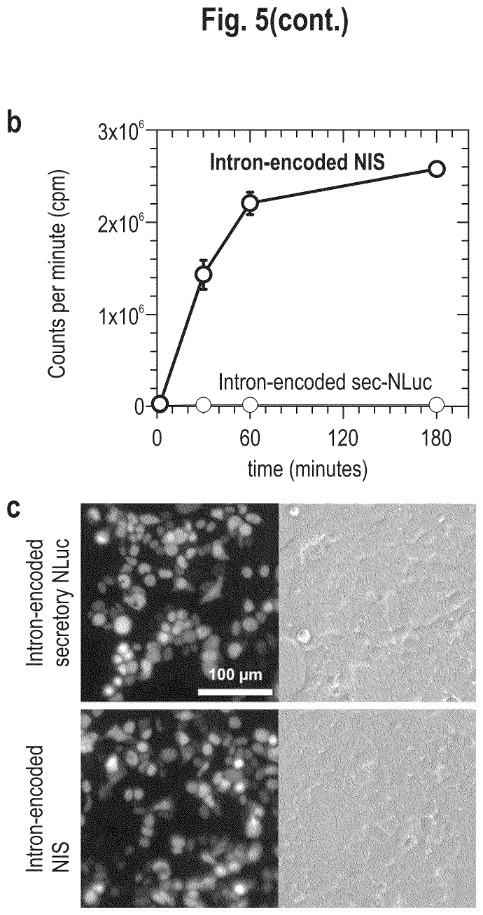

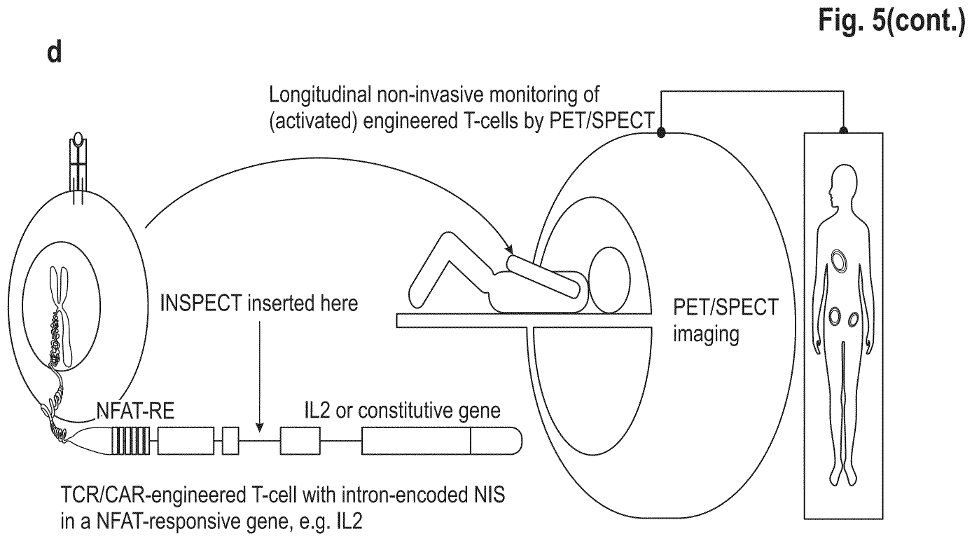

After optimization of the intron-encoding capability of the system, the inventors wondered if more complex proteins could be intronically expressed. They selected the sodium-iodide symporter (NIS alias SLC5A5), which was integrated into the membrane at the endoplasmic reticulum, as a complex IEP. The expression of NIS could be monitored by measuring the accumulation of radioactive iodine (131I−), which was normally not absorbed by non-thyroid cells (FIG. 5a). Cells transfected with the intron-encoded NIS showed a dramatic incubation-time-dependent increase in accumulated radioactivity (FIG. 5b), which shows that complex multipass transmembrane proteins can also be encoded in the intron.

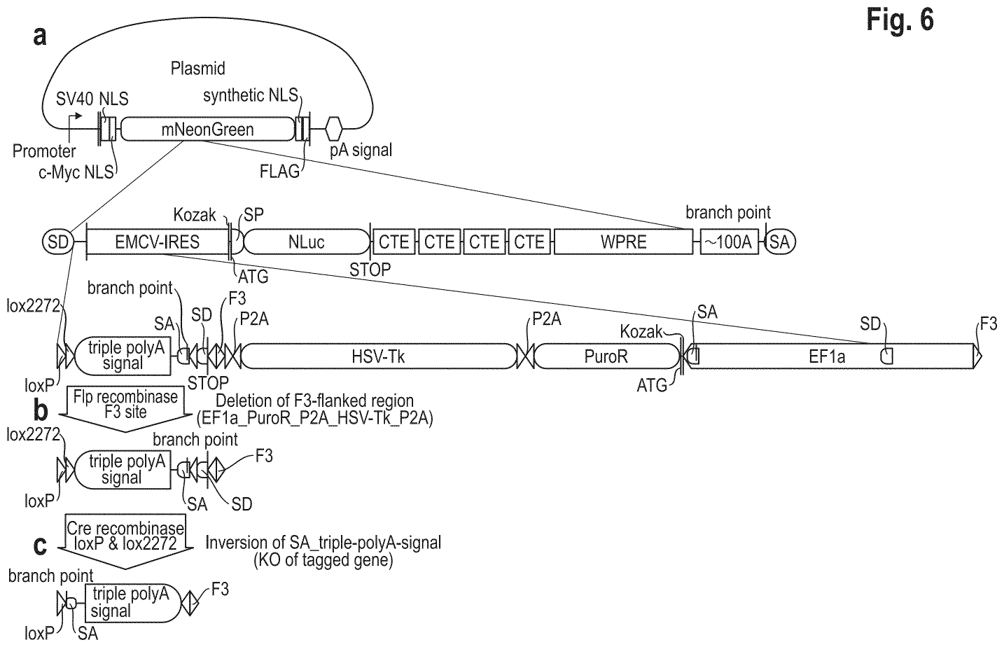

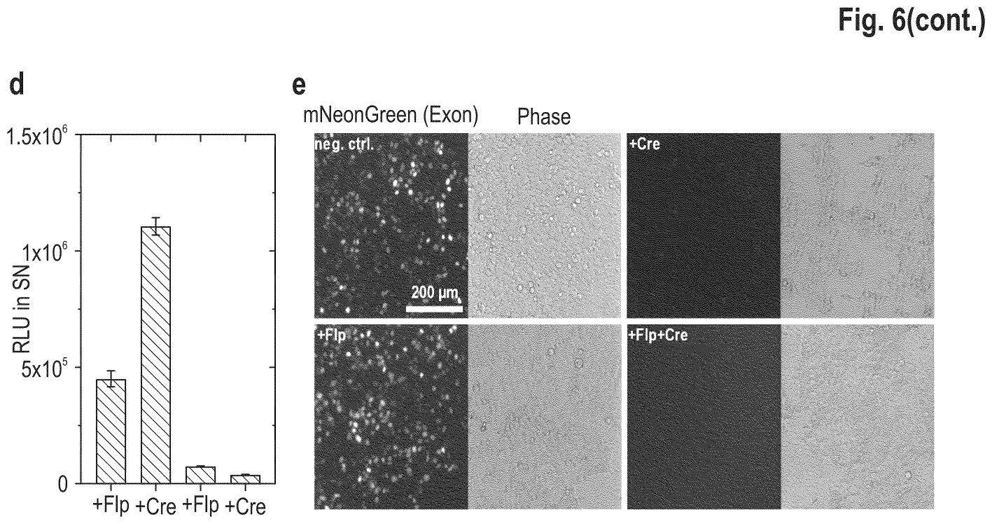

The inventors integrated a knock-out-switch into the genetic system in a non-invasive way. The inventors tested this KO-switch in the exonic mNeeonGreen-NLS system and co-expressed Cre or FIp recombinases to benchmark the KO-efficiency (FIG. 6a). Upon FIp recombinase expression, both the mNeonGreen and the NLuc activity in the supernatant increased, which can be explained by the excision of the inverted EF1α-driven cassette, the transcriptional interference of the CAG-driven mNeonGreen by the EF1α-promoter does not occur anymore (FIG. 6b, d, e). Upon Cre recombinase expression, the exonic mNeonGreen signal and the intronic NLuc signal was dramatically decreased, indicating an efficient Cre-mediated off-switch (FIG. 6c, d, e).

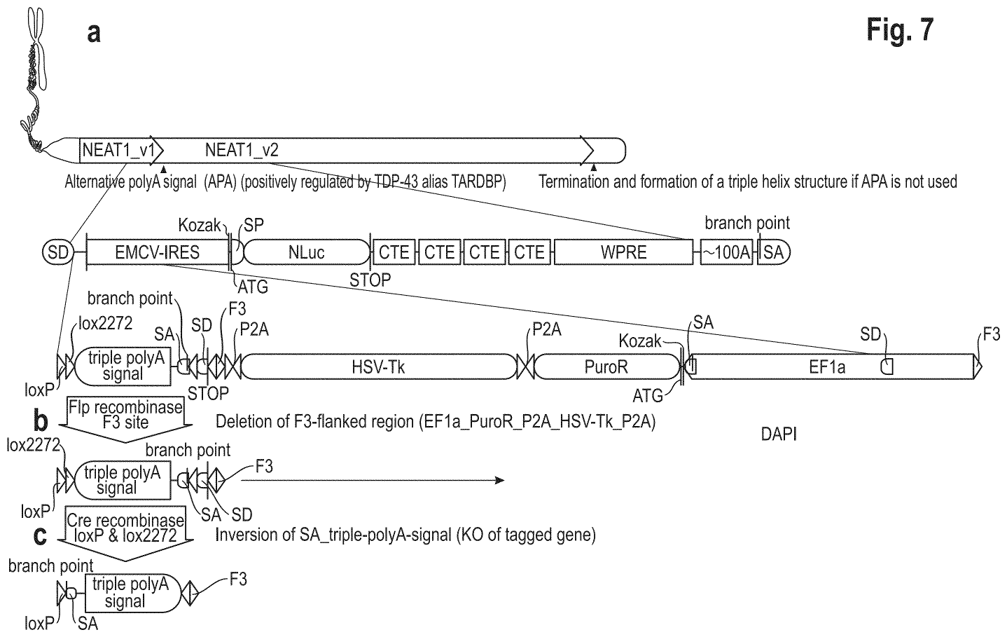

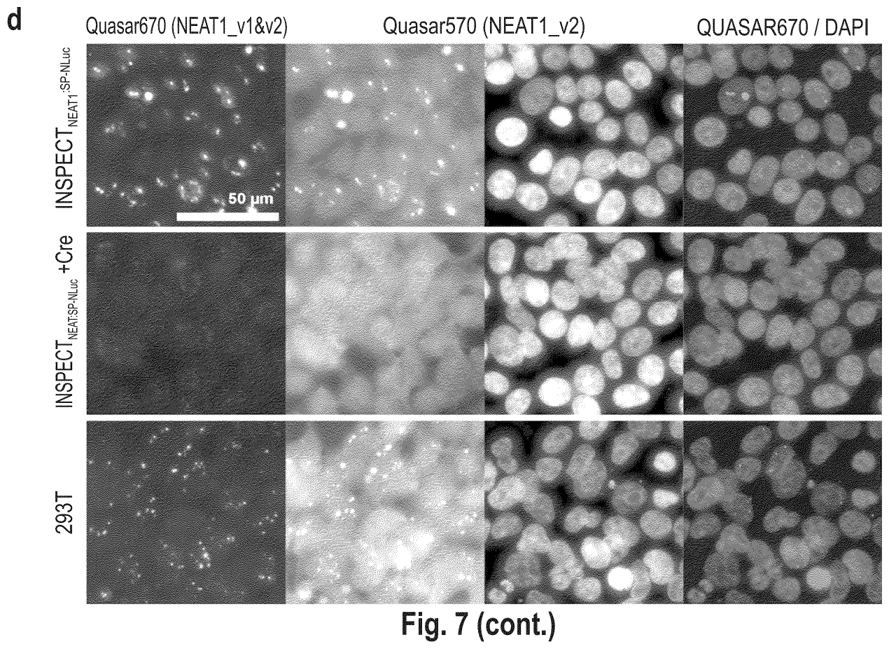

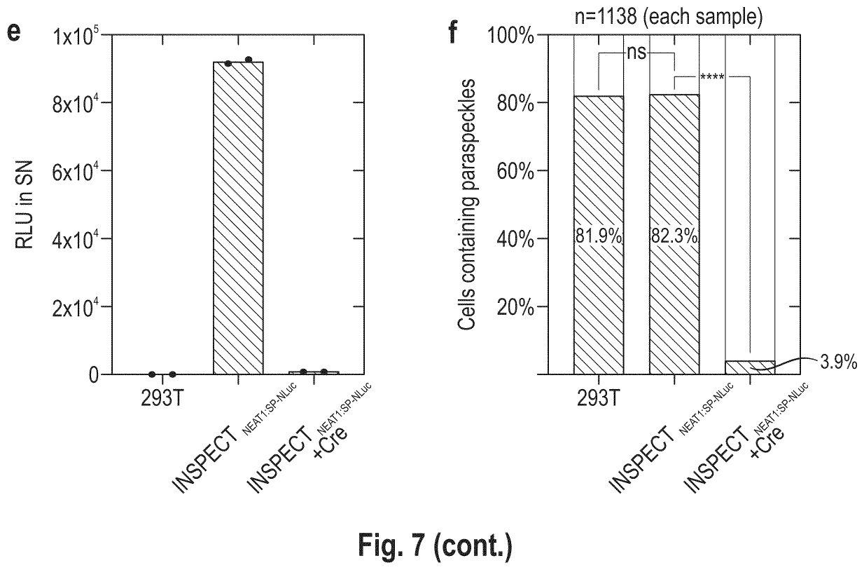

Ultimately, the inventors wanted to show that they can transcriptionally couple a non-coding RNA non-invasively via the system to a secretory luciferase and knock it out afterward via Cre recombinase. They selected the long non-coding RNA (lncRNA) NEAT1. The inventors introduced the reporter SP-NLuc using CRISPR/Cas9 into the shared region of NEAT1_v1 and NEAT1_v2 (FIG. 7a). After successful knock-in, selection (puromycin), FIp-mediated cassette excision (FIG. 7b) and counter-selection (Ganciclovir) only homozygous clones were used for further analysis. A subclone with homozygous NEAT-KO was also created by transfecting a homozygous clone with a plasmid expressing Cre recombinase (FIG. 7c). TDP-43, which usually shows an increased expression in stem cells, stimulating the premature polyadenylation of NEAT1_v1, thus exclusively expressing v1. If the level of TDP-43 decreases during cell differentiation, NEAT1_v2 is also expressed more frequently because the alternative poly(A) site (APA) of NEAT1_v1 is used less. Since NEAT1_v2 is an essential part of so-called nuclear bodies called paraspeckles (an agglomeration of NEAT1 RNA and sequestered proteins), differentiation also will induce paraspeckle formation. Using smFISH analysis, the inventors showed that both the reporter clone and unmodified HEK293T cells have paraspeckles, but not the subclone with Cre, where the inverted SA_3×poly(A) signal was flipped in its sense direction. Consequently, the NLuc signal was also barely detectable in the KO clone, clearly demonstrating a transcriptional coupling between the gene and that of the intron-encoded reporter. At the same time, it was also shown that the protein encoded in the intron has no relevant upstream promoter-like sequences that generate false-positive background luciferase activity. Otherwise, a residual signal would be evident despite Cre recombinase. The quantification of the images of the reporter clone and unmodified HEK293T cells (representative examples shown in FIG. 7d) also showed that the number of paraspeckles-containing cells remained unchanged (FIG. 7f).

When summarizing the state-of-the-art for which we index the version of the constructs as version0 (v0) (see section A) and then compare it with section B) given below, the improvements of version1 (v1) are shown, which are: i) monitoring of the long non-coding RNA NEAT1, without disrupting the nuclear structures it forms (paraspeckles), ii) monitoring the coding gene IL2, important in T-cells, with a translated reporter enzymes, and iii) a secreted RNA reporter/barcode, for which the inventors developed a minimal-export unit, based on the viral protein gag, which suppresses secretion of endogenous RNAs and instead exports the promoter-specific (because of the insertion in the intron) RNA barcode. This method to couple a designer RNA barcode to a gene of choice (by inserting it into an appropriate intron), exporting it out of the nucleus via the features described in v1 and then exporting it out of the cell via a minimal gag exporter and the appropriate RNA aptamer handle on the RNA barcode is clearly distinct and different from WO 2020/205681, which focuses on the secretion of “natural biomolecules” out of the cell.

A) State of the art is characterized by the following: 1. The constructs containing a “synthetic intron (SI)” (defined as splice donor (SD), branch point (BP) and splice acceptor (SA)) can be reprogrammed to be interpreted as a “simulated exon” by the cellular machinery and is exported (instead of degradation) to the cytosol. 2. A reporter CDS downstream of an “Internal Ribosome Entry Site (IRES)” is inserted to enable 5′-cap and 3′-poly(A) independent translation, since an intron does neither contain a 5′-cap nor a 3′-poly(A) tail. This moiety will be called IRES:reporter-CDS in the following. 5′ and 3′-insertions will be used in the following to describe, where RNA export, or stabilization elements, or translation enhancing elements will be inserted relative to the IRES:reporter-CDS entity mentioned in (2.). 3. The inventors of the present invention show herein that CTE combined with WPRE, and a genetically encoded poly(A) tail, inserted into the 3′ region of the SI, enabled the readout of gene expression of the lncRNA NEAT1. This version will be defined from now on as version 0 (v0). 4. The inventors of the present invention show herein that insertion of v0 showed morphological similar sized paraspeckles compared to the WT. (Reminder: Paraspeckles are made of the lncRNA NEAT1 plus many other RNA-binding proteins (RBPs)). 5. Thus, v0 was the first version of the inventors of the present invention, which showed the capability of such a reprogrammed intron to monitor non-coding genes, such as NEAT1.

B) The following improvements were made with respect to the present invention as described herein (in in comparison to the prior art).

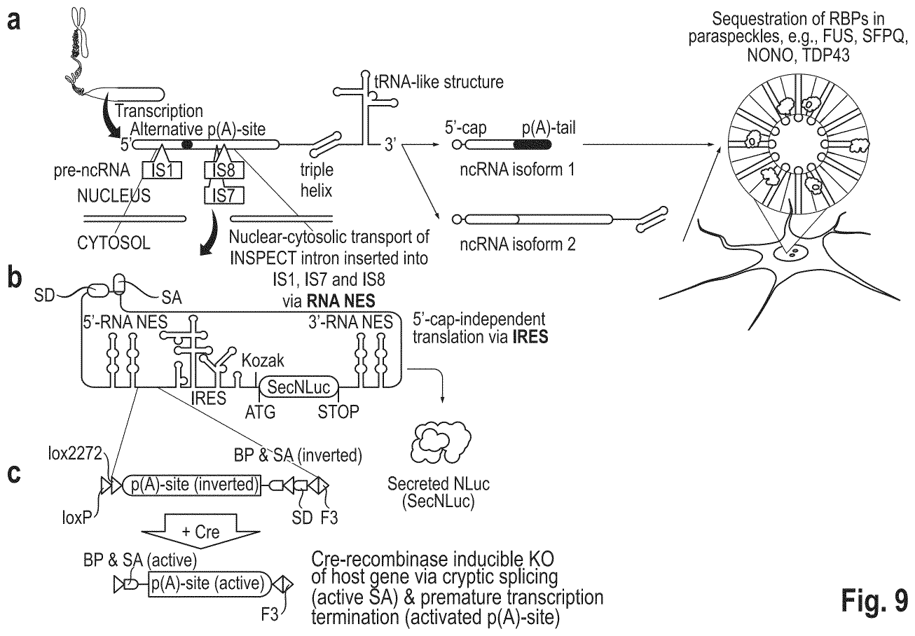

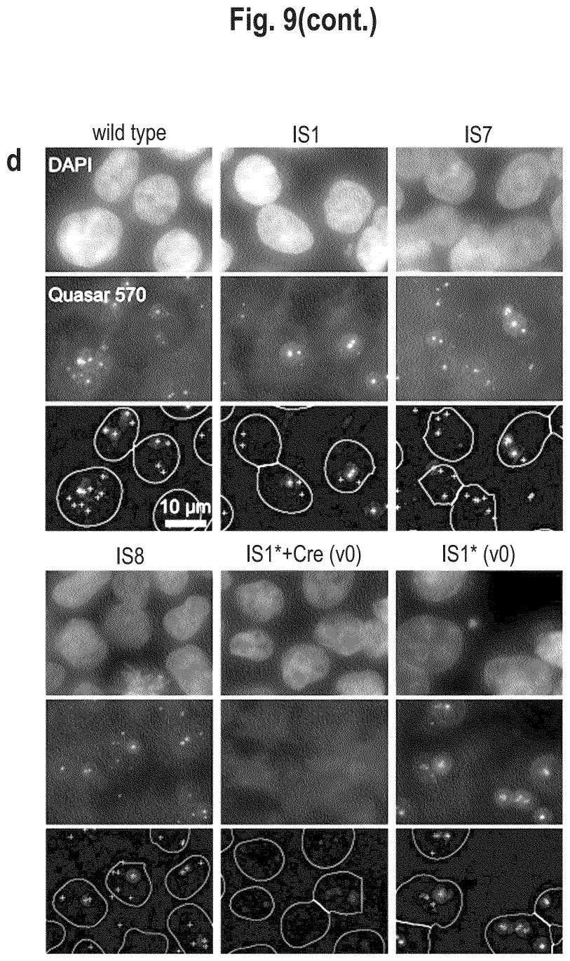

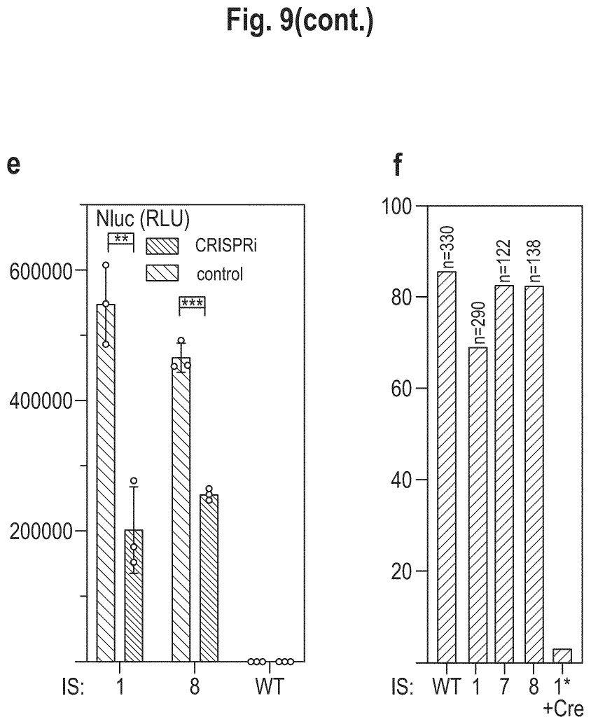

1. The inventors of the present invention realized after detailed analysis that the paraspeckles were somewhat bigger and not as roundish compared to WT cells (see FIG. 9d; v0 vs. WT).

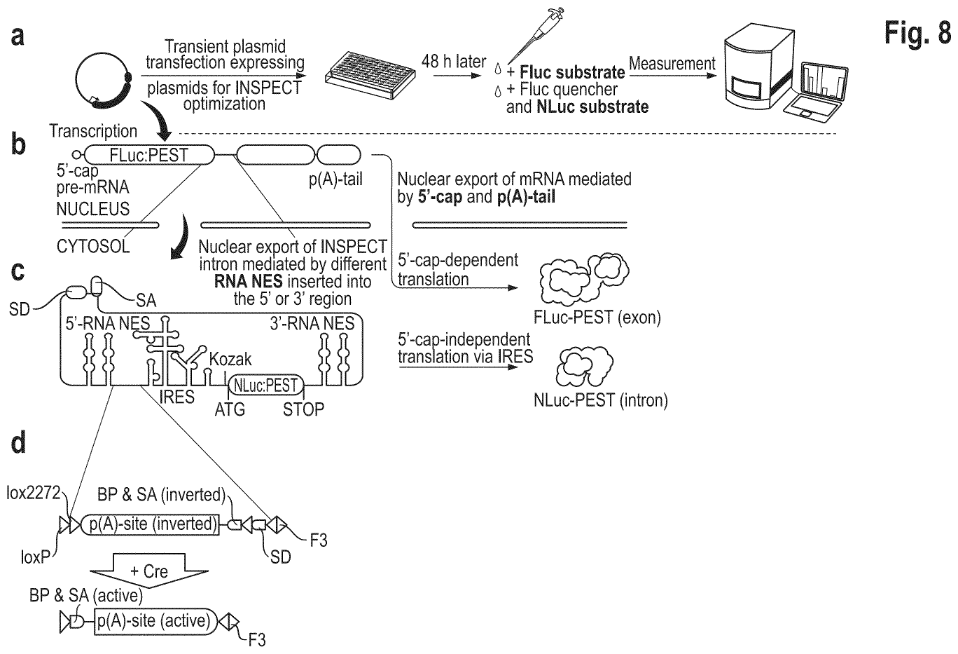

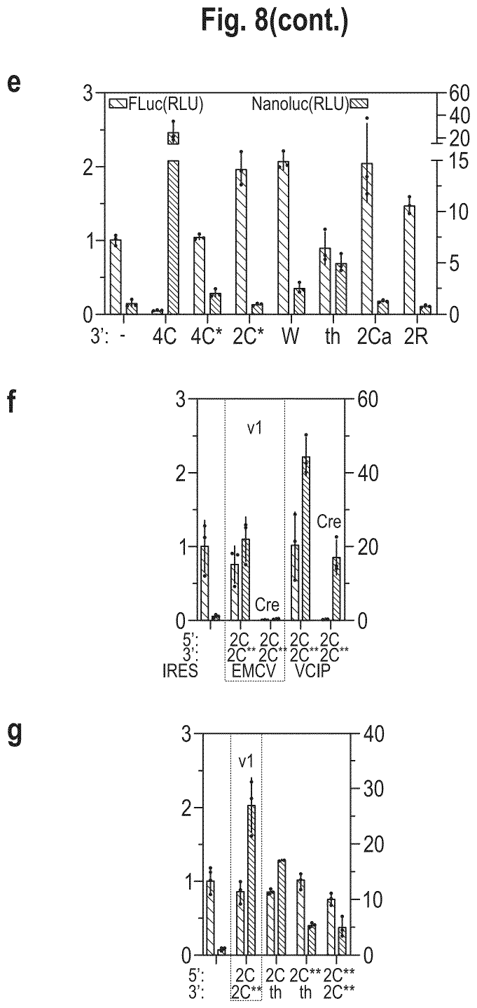

2. Further improvement of the constructs (v1 reporter) for nuclear export and cap-independent was achieved, to further improve the minimal invasiveness for monitoring non-coding genes: a) The inventors of the present invention setup a dual-luciferase readout to find a combination of different elements which does not induce cryptic splicing (=non-intended splicing of sites, which resembles splice-consensus-like sites) and maintain its ability to mediate efficient export of this SI. In this optimization assay (see FIG. 8a), firefly luciferase (FLuc) reports the correct splicing of the exonic part of the pre-mRNA, whereas NanoLuc luciferase (NLuc) reports the successful export and translation of the SI. Thus, high FLuc values indicate the correct splicing of the exon, low FLuc values on the contrary indicate that splicing did not work as intended, e.g., because of cryptic splice sites. High NLuc values indicate efficient export of the SI and efficient IRES-dependent translation of the reporter-CDS part. The aim of the assay was to find a combination of elements that maintain the same splicing efficiency as a reference control construct containing no elements at all beside a SI plus the IRES:reporter-CDS moiety, but has maximal efficiency regarding the expression of the SI-embedded reporter-CDS (high NLuc). b) See again definition of 5′- and 3′ insertion sites in A) 2 to interpret the FIG. 8e-g. The inventors of the present invention inserted different elements into the 5′- and 3′ region and also tested multiple combinations of promising variants. C: CTE sequence; C*: Mutant of C; C**: Another mutant of C. W: WPRE; the triple helix taken from mouse Malat1 lncRNA stabilizes the 3′-end of RNAs; Ca: CAE (cytoplasmic accumulation element) from xenotropic murine leukemia virus; R: m26 mutant from RTE from rodent intracisternal A-particles. EMCV: EMCV-IRES; VCIP: VCIP IRES. Numbers indicate tandem insertions of the same element, e.g., 2C indicate 2× tandem insertions of the C element. c) FIG. 8e shows that compared to the reference construct without any insertions, within the SI, besides the IRES:reporter-CDS, the 3′-4C insertion (used in v0; v0 also contains in addition WPRE) induced massive reduction in FLuc signal indicating a high amount of wrongly spliced exonic mRNA (FLuc). In contrast, RNA-stabilizating elements such as a 3′-th could enhance the NLuc (intron-encoded protein) signal without changing the FLuc signal (exon-encoded protein). d) c described directly above, which induced aberrant splicing when inserted into the 3′-region, was beneficial, when inserted in tandem into the 5′-site (5′-2C) in combination with a mutant version (C**) inserted also in tandem into the 3′-region (see FIG. 8f). This also showed the non-obviousness of the system, due to position effect within an intron. Also, empirical screenings had to be performed to find an optimal system. e) The inventors of the present invention used a Cre-recombinase-mediated KO-switch (FIG. 9d) to validate that the NLuc activity was not the result of some cryptic promoter activity encoded on the IRES within the SI. As shown in FIG. 8f, VCIP IRES showed substantial NLuc activity even in the presence of Cre-recombinase activity, indicating that not all IRES can be used to create a faithful intron-encoded reporter system. This also supports the “non-obviousness” of the method of the present invention, because not any IRES can be used. f) An SI equipped with 5′-2C together with 3′-2C** together with an EMCV-IRES to drive the reporter CDS are declared as v1 and were used in FIG. 9 to insert into insertion sites 1 (IS1), IS7, and IS8 of the lncRNA gene NEAT1. g) As one can see, when zooming into the paraspeckles, v0 (that contained 3′-4C insertions together with 3′-W) showed not so roundish and more diffuse paraspeckles compared to wild type cells in IS1 (FIG. 9d, IS1* (v0)). IS1 with v1 on the contrary (also IS7 and IS8) showed morphologically undistinguishable paraspeckles compared to wild type cells (FIG. 9d, IS1, IS7, and IS8). h) The inventors of the present invention also created supporting data of the described reporter system correlating with the expression of NEAT1. The inventors of the present invention performed CRISPRi (using dCas9:transcriptional-repressor) targeted against the NEAT1 promoter (5′-region of the NEAT1 gene) and observed an CRISPRi-dependent reduction in NLuc signal for both, v1 inserted into IS1 and v1 inserted into IS8 (FIG. 8e).

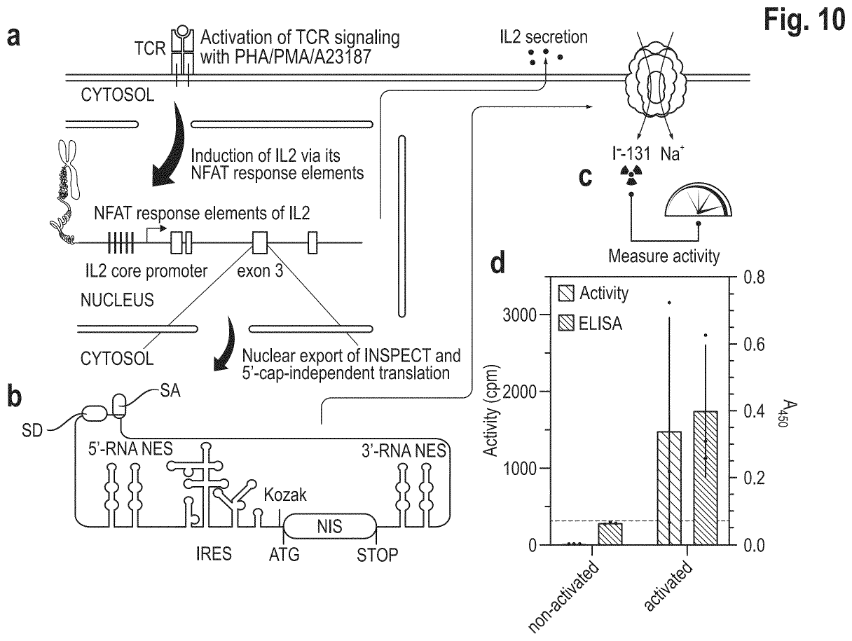

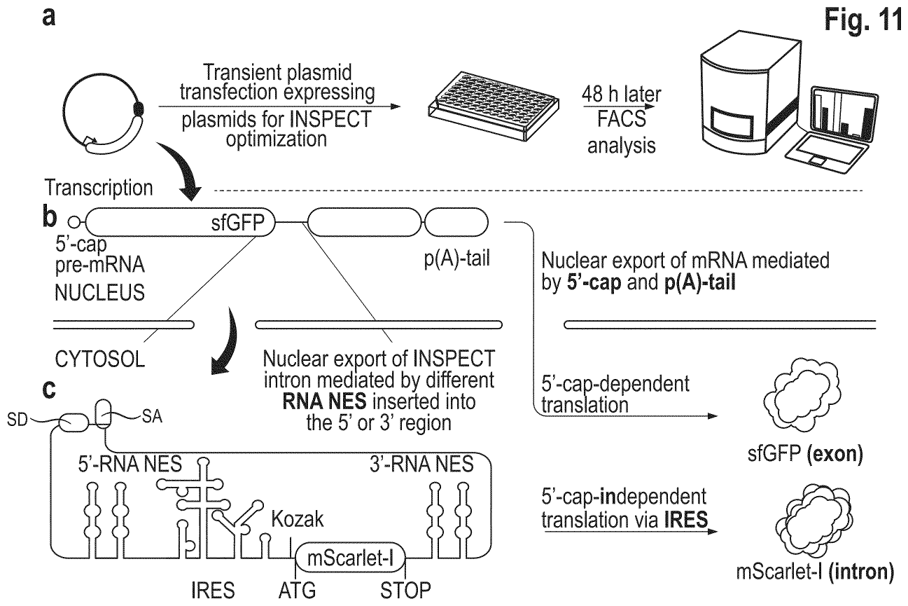

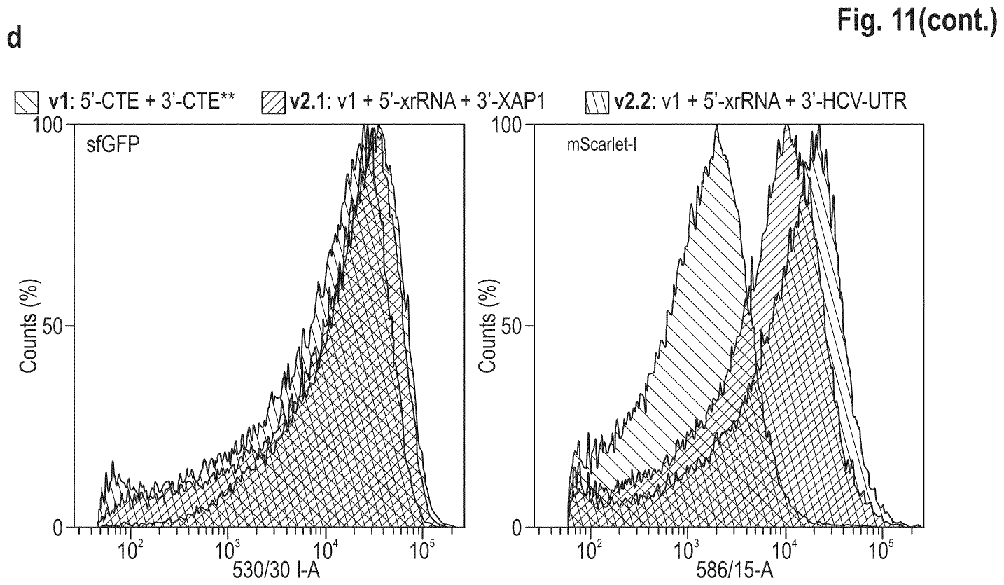

3. The v1 reporter system can also be inserted into constitutive exons within coding genes such as, IL2 in the T lymphocyte cell line Jurkat E6-1. a) Here, the inventors of the present invention also showcased that large reporter genes, such as the sodium iodide symporter (NIS, ˜2 kbp CDS) (in contrast to the relatively small NLuc, encoded by ˜0.5 kbp) can be non-invasively nested into the v1 SI instead of NLuc (FIG. 10a,b). NIS is used as a novel reporter gene for molecular imaging since it can accumulate iodide radioisotopes, which can read out by PET/SPECT-imaging and by gamma counters. b) After T cell signaling (stimulation with PHA/PMA/A23187, FIG. 10a), the cytokine IL2 was rapidly induced and was then subsequently secreted into the supernatant. Using the v1 reporter system equipped with NIS (FIG. 10b), the inventors of the present invention showed that the engineered cells were still responsive to TCR stimulation and were able to secrete IL2 after stimulation (FIG. 10d, ELISA against IL2). In addition, TCR stimulation also induced the expression of the intron-encoded NIS, as measured by a gamma counter, which detects the accumulation of the gamma emitter I-131⁻ ions in the cells (FIG. 10d, measured activity by the gamma counter). c) This example showcased the versatility of the method of the present invention to also equip large reporter genes without interfering with the function of the host gene. d) The inventors of the present invention sought to further boost the “coding capacity” of the intron to encode proteins in v1 and used a fluorescence-based read-out to measure the exonic (sfGFP, green fluorescence) expressed protein and the intronic (mScarlet-I, red fluorescence) expressed protein level via FACS analysis (FIG. 11a-c). This system had the same principle as the optimization assay using FLuc (exon-encoded protein) and NLuc (intron-encoded protein) from FIG. 9. As shown in FIG. 11d, the intron-encoded protein expression level could be increased by 5-fold (v.2.1) or 10-fold (v2.2) compared to v1 by the insertion of additional elements in the 5′- or 3′-region within the SI. Both, v2.1 and v2.2 contained additional 5′-xrRNA elements, which protected its 5′-end by exonucleases and v2.1 a 3′-XAP1 element, which was bound by the nuclear export factor XPO1 (CRM1) and thereby improved the export of the SI, whereas v2.2 contained the 3′-UTR of Hepatitis C virus (3′-HCV-UTR), which supports the translation.

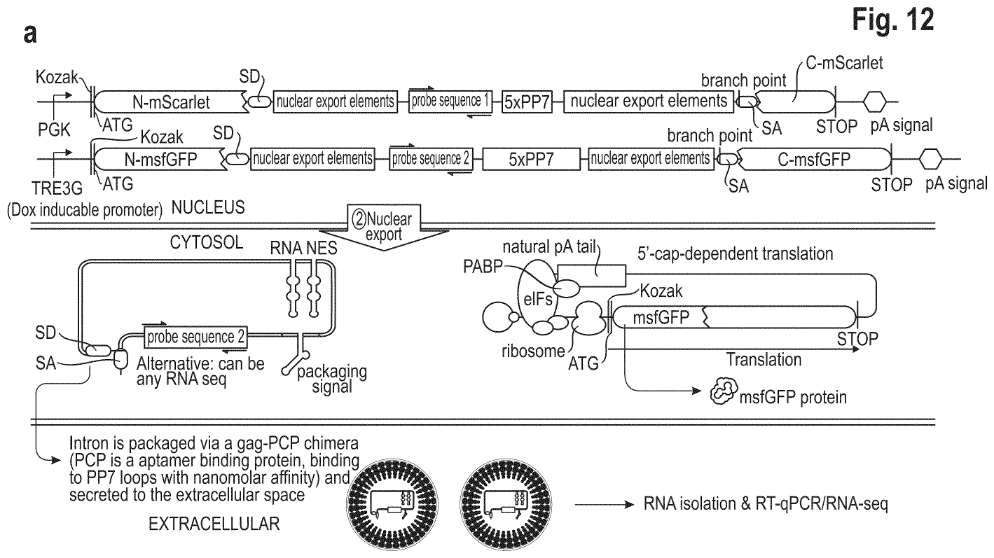

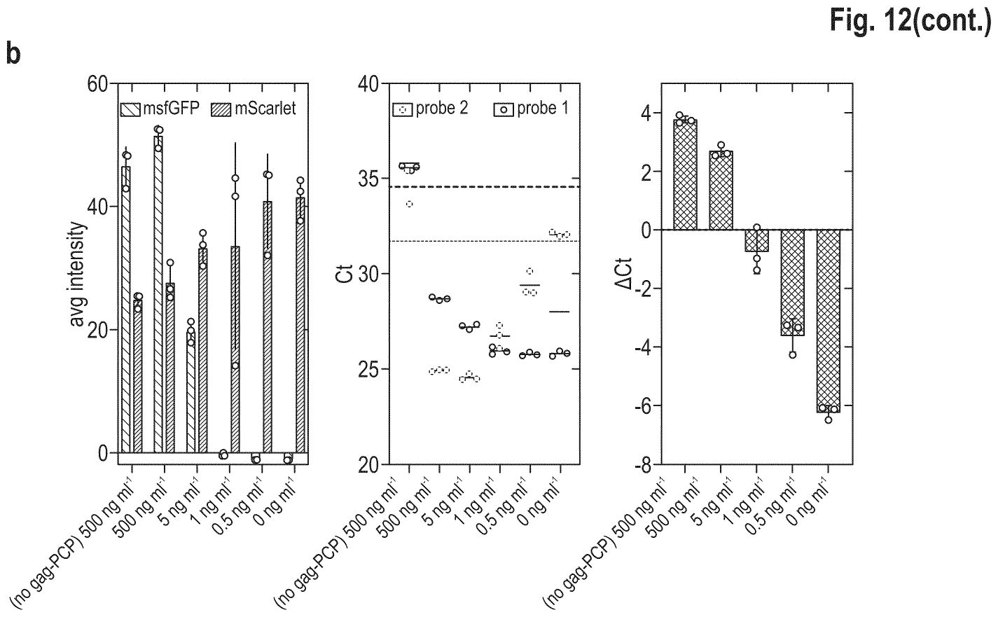

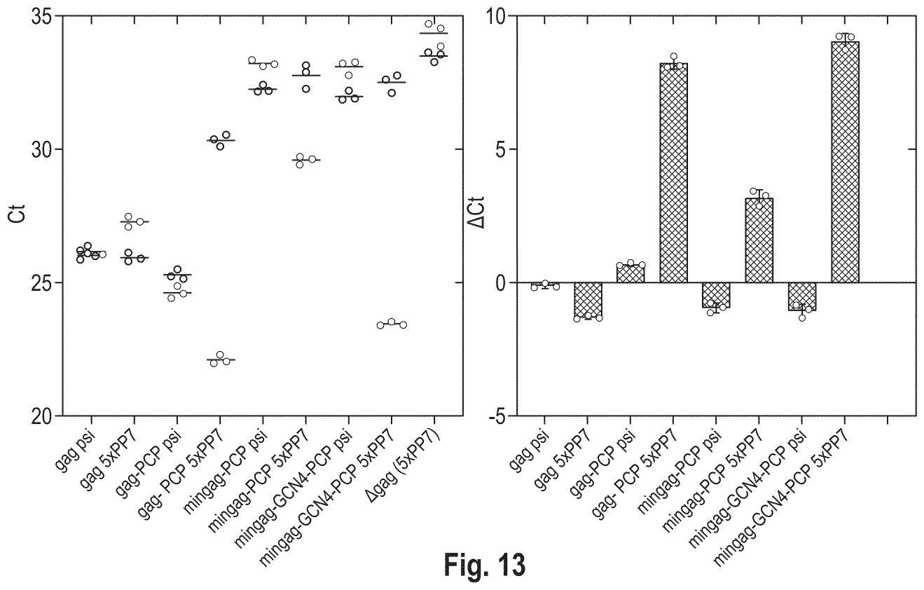

4. The intron-embedded transcripts that were exported from the nucleus could also be exported out of the cell (instead of being translated) such that they could be detected via sequence-specific methods. a) To achieve an efficient export from the cell as opposed to a translation, the inventors of the present invention removed the IRES:reporter-CDS and added instead a unique RNA-snippet (can be defined as expressible nucleic acid barcode in the following, or in short barcode). As proof of concept, the inventors of the present invention created two plasmids, one constitutively expressing mScarlet-I (Pgk1 promoter driven) and one expressing sfGFP in the presence of doxycycline (TRE3G promoter driven) (FIG. 12a). Both fluorescent proteins were intersected by the SI equipped with elements from v1 and a unique RNA-barcode instead of a reporter CDS and additional 5×PP7 aptamer motifs (aptamers are RNA motifs that are recognized by specialized RNA-binding proteins recognizing these motifs (FIG. 12a). When co-transfecting HEK293T cells together with a gag-chimera (derived from HIV-1), where the ZF2 zinc finger domain was replaced by the aptamer binding protein PCP (gag-PCP), which binds to PP7 loops, it was expected that after the nuclear export of the intron mediated by the RNA nuclear export motifs, these PP7 loops were recognized by the gag-PCP chimera and were packaged and secreted into the supernatant as virus-like particles (VLPs), which was readily detected via RNA-based methods after suitable RNA-purification methods (FIG. 12a). b) After transfection of the plasmids (plasmid encoding constitutively expressed mScarlet-I, plasmid encoding doxycycline-inducible sfGFP via TRE3G promoter, plasmid encoding Tet-On 3G, which controls the TRE3G promoter, and a plasmid encoding the gag-PCP chimera), the cells were induced with different concentrations of doxycycline. After further 24 h of induction, mScarlet-I and sfGFP were quantified according to their fluorescence via fluorescence microscopy and the supernatant of the cells was collected in addition subsequently for RNA-extraction and RT-qPCR. c) Shown in FIG. 12b (left charts) are the mean fluorescence intensity (MFI) of the imaged cells in the presence of different doxycycline induction concentrations. sfGFP was massively induced with 500 and 5 ng/μL doxycycline and were not anymore detectable with lower induction concentrations. In contrast, mScarlet fluorescence remained relatively stable and was brighter with less induction agent since the expression machinery was mainly expressing sfGFP during high doxycycline concentrations. This could also be observed via sampling of the supernatant and downstream RNA-analysis of the intronic RNA barcode sequence, representing the expression of sfGFP or mScarlet-I (FIG. 12b, middle chart). Here, the inventors of the present invention could see low ct values for RT-qPCR reactions, detecting the RNA barcode snippet engrafted into the intron representing sfGFP (low ct values=high RNA abundance, high ct values=low RNA abundance), indicating its high expression level, whereas without induction, high ct-values could be observed. Also, for mScarlet-I, an inverse trend was observed. Most importantly, maximum induction, but without gag-PCP expressed, only basal ct-values (high ct values) could be measured indicating the cytosolic introns cannot be readily secreted by their own into the supernatant and require gag-PCP to export these barcoded cytosolic introns. On the right bar chart of FIG. 12b, one can see the Oct plots for the corresponding conditions, where the ct values are plotted as a difference between both ct-values, representing mScarlet-I and sfGFP. High act-values indicate relatively high sfGFP expression level compared to mScarlet-I. Low or negative Dct values indicate relatively low sfGFP expression level compared to mScarlet-I. d) To make the gag-PCP chimera-mediated export of cytosolic aptamer-tagged introns more specific, the inventors of the present invention also created minimal versions of gag by truncating unnecessary elements of gag and only maintained the domains being important for gag-assembly and budding. The inventors of the present invention used here a two-plasmid system expressing two different proteins (thick and thin-lined circles), where the plasmid encoding a protein (thin-lined circles) with 5×PP7 loops in the 3′-UTR tagged mRNA and where a control plasmid encoding a different protein (thick-lined circles) was not tagged any sequence in the 3′-UTR and therefore was not exported by gag-PCP. As an additional control plasmid, the inventors of the present invention also tagged the 3′-UTR with the psi elements from HIV-1 which is not recognized by gag-PCP due to the zinc finger deletions. The aim of this experiment was to check how specific a PP7-loop-tagged RNA is exported compared to untagged or psi-tagged mRNA. e) Without any gag or gag-PCP (Δgag), only high ct-values could be measured for RNA-extracted from the supernatant, transfected with the indicated plasmid. This indicated only spurious presence of RNA in the supernatant, when there is no gag expressed. However, expression of non-PP7-loop-tagged RNA together with gag or gag-PCP resulted in the export of all RNA species (low ct values compared to Δgag). Expression of PP7-tagged RNA resulted in even lower ct-values for this PP7-tagged RNA species and an increase in ct-values of the untagged RNA species indicating less unspecific export then the tagged RNA substrate is available. In other words, these results indicated that gag-PCP can mediate specific export of PP7-tagged RNAs, but in the absence of its substrate, gag-PCP (and also gag) is exporting all other RNA species regardless of their sequence (FIG. 13). To improve the specificity, the inventors of the present invention created truncated gag versions (minigag) and added a dimerizing domain GCN4 (a coiled coil homodimer) (which replaced a more complex dimerizing motif of original gag) together with PCR In stark contrast to gag-PCP, minigag-GCN4-PCP and minigag-PCP did not show any unspecific export of untagged RNA-species (no PP7 loops) (high ct values for conditions with minigag-(GCN4)-PCP combined with psi) even in the absence of any PP7-tagged RNA. When the 3′-UTR was tagged with 5×PP7 loops, a massive reduction could be observed for the same RNA species (thin-lined circles), while the untagged RNA species still had nearly the same high ct-values (thick-lined circles) as the gag control for minigag-GCN4-PCP. Deletion of GCN4 (miniGag-PCP) decreased the efficient export of the PP7-tagged RNA species (higher ct compared to minigag-GCN4-PCP, thin-lined circles). In summary, the inventors of the present invention were able to maintain the high specificity of PCP-PP7 interaction and removed the unspecific RNA-interaction from gag by using a minimal truncated version of gag combined with a specific aptamer binding protein (PCP). Instead of PCP-PP7 interaction, also other RNA-RBP interactions can be used, such as a MS2-MCP, Cas9-sgRNA, Cas12a-crRNA, Cas13a/b/c/d/etc.-crRNA etc. f) The point 12 and 13 describes how an abstract information can be encoded within a synthetic intron (SI) equipped with nuclear export elements as described above, but not necessary with the translation unit composed of IRES-reporter CDS. Instead, an RNA-aptamer has to be introduced into the SI and a VLP-forming system (in this case gag VLPs) has to be co-introduced into the cell to readily grab the cytosolic intron with the barcode information and then subsequently transfer it via viral budding into the supernatant. The key feature is again the non-invasiveness of the method of the present invention, which would be not possible using full-gag chimeras since it would secrete also untagged RNA species as shown in FIG. 13. Only the delicate combination of an aptamer binding protein with a minimal gag (chimera of a minimal gag with an RNA-binding protein as not described in the prior art) allowed us to non-invasively only secret the cytosolic intron-species specifically into the supernatant.

Thus, the present invention relates to a method for detecting a nucleic acid construct or part thereof and/or detecting the expression product of the nucleic acid construct or part thereof,

wherein the method comprises inserting a nucleic acid construct or part thereof into an intron or a synthetic intron,

wherein the nucleic acid construct comprises:
a. at least one heterologous nucleic acid sequence, which does not encode a protein;
   at least one nucleic acid sequence for transcription of the nucleic acid construct or part thereof, and
   at least one nucleic acid sequence for exporting the nucleic acid construct or part thereof out of the nucleus,
or
b. at least one heterologous nucleic acid sequence, which encodes a protein,
   at least one nucleic acid sequence for transcription of the nucleic acid construct or part thereof,
   at least one nucleic acid sequence for preventing degradation of the nucleic acid construct or part thereof,
   at least one nucleic acid sequence for exporting the nucleic acid construct or part thereof out of the nucleus, and
      at least one nucleic acid sequence for translation of the nucleic acid construct or part thereof

In one embodiment, the method of the present invention relates to a method for detecting a nucleic acid construct or part thereof,

wherein the method comprises inserting a nucleic acid construct or part thereof into an intron or a synthetic intron,

wherein the nucleic acid construct comprises:
a. at least one heterologous nucleic acid sequence, which does not encode a protein;
   at least one nucleic acid sequence for transcription of the nucleic acid construct or part thereof, and
   at least one nucleic acid sequence for exporting the nucleic acid construct or part thereof out of the nucleus,
or
b. at least one heterologous nucleic acid sequence, which encodes a protein,
   at least one nucleic acid sequence for transcription of the nucleic acid construct or part thereof,
   at least one nucleic acid sequence for preventing degradation of the nucleic acid construct or part thereof,
   at least one nucleic acid sequence for exporting the nucleic acid construct or part thereof out of the nucleus, and
      at least one nucleic acid sequence for translation of the nucleic acid construct or part thereof.

In one embodiment, the method of the present invention relates to a method for detecting the expression product of the nucleic acid construct or part thereof,

wherein the method comprises inserting a nucleic acid construct or part thereof into an intron or a synthetic intron,

wherein the nucleic acid construct comprises:
a. at least one heterologous nucleic acid sequence, which does not encode a protein;
   at least one nucleic acid sequence for transcription of the nucleic acid construct or part thereof, and
   at least one nucleic acid sequence for exporting the nucleic acid construct or part thereof out of the nucleus,
or
b. at least one heterologous nucleic acid sequence, which encodes a protein,
   at least one nucleic acid sequence for transcription of the nucleic acid construct or part thereof,
   at least one nucleic acid sequence for preventing degradation of the nucleic acid construct or part thereof,
   at least one nucleic acid sequence for exporting the nucleic acid construct or part thereof out of the nucleus, and
   at least one nucleic acid sequence for translation of the nucleic acid construct or part thereof.

In one embodiment, the method of the present invention relates to a method for detecting a nucleic acid construct or part thereof, wherein the method comprises inserting a nucleic acid construct or part thereof into an intron or a synthetic intron, wherein the nucleic acid construct comprises:
at least one heterologous nucleic acid sequence, which does not encode a protein;
at least one nucleic acid sequence for transcription of the nucleic acid construct or part thereof, and
at least one nucleic acid sequence for exporting the nucleic acid construct or part thereof out of the nucleus.

In one embodiment, the method of the present invention relates to a method for detecting a nucleic acid construct or part thereof and/or detecting the expression product of the nucleic acid construct or part thereof, wherein the method comprises inserting a nucleic acid construct or part thereof into an intron or a synthetic intron,

wherein the nucleic acid construct comprises:
at least one heterologous nucleic acid sequence, which encodes a protein,
at least one nucleic acid sequence for transcription of the nucleic acid construct or part thereof,
at least one nucleic acid sequence for preventing degradation of the nucleic acid construct or part thereof,
at least one nucleic acid sequence for exporting the nucleic acid construct or part thereof out of the nucleus, and
at least one nucleic acid sequence for translation of the nucleic acid construct or part thereof.

In one embodiment, the present invention relates to a method for detecting a nucleic acid construct or part thereof and/or detecting the expression product of the nucleic acid construct or part thereof,

wherein the method comprises inserting a nucleic acid construct or part thereof into an intron or a synthetic intron,

wherein the nucleic acid construct comprises:
a. at least one heterologous nucleic acid sequence, which does not encode a protein;
   at least one nucleic acid sequence for transcription of the nucleic acid construct or part thereof, and
   at least one nucleic acid sequence for exporting the nucleic acid construct or part thereof out of the nucleus,
or
b. at least one heterologous nucleic acid sequence, which encodes a protein,
   at least one nucleic acid sequence for transcription of the nucleic acid construct or part thereof,
   at least one nucleic acid sequence for preventing degradation of the nucleic acid construct or part thereof,
   at least one nucleic acid sequence for exporting the nucleic acid construct or part thereof out of the nucleus, and
   at least one nucleic acid sequence for translation of the nucleic acid construct or part thereof,
      and wherein the method comprises transcribing the heterologous nucleic acid sequence together with an endogenous gene of interest and detecting the same heterologous nucleic acid sequence. It is preferred in this embodiment, that the at least one nucleic acid sequence for translation of the nucleic acid construct or part thereof is an open reading frame or internal ribosomal entry site for translation of the heterologous nucleic acid sequence which encodes a protein.

In the present invention, the term “detecting” means to discover or identify the presence or existence of a sequence, which can be, for example, a (non-coding) RNA or a protein of interest. The term “detecting” means specifically, in the context of the present invention, to discover or identify the presence or existence of a nucleic acid construct or part thereof and/or the expression product of the nucleic acid construct or part thereof.

In the present invention, the term “nucleic acid construct” describes a combination of DNA or RNA sequences, which may or may not be functionally different, or carry information and can be linked together directly or through linker parts. Such a genetic construct is also known as genetic cassette. The separate compounds of this construct are defined as nucleic acid sequences and are described in the following. This includes, but is not limited to, (a) nucleic acid sequence(s) for transcription of the nucleic acid construct or part thereof, (a) nucleic acid sequence(s) for exporting the nucleic acid construct or part thereof out of the nucleus, (a) nucleic acid sequence(s) for preventing degradation of the nucleic acid construct or part thereof, and (a) nucleic acid sequence(s) for exporting the nucleic acid construct or part thereof out of the nucleus. The mentioned nucleic acid construct contains in each case at least one heterologous nucleic acid sequence, which may be for example non-coding or coding. In some preferred embodiments, (a) sequence(s) to enable cap-independent translation of the nucleic acid construct may also be present. All of the stated parts of the nucleic acid construct are explained in more detail somewhere herein.

In the present invention, the term “expression” describes throughout the whole description, a biological process in which the information of a DNA part is converted into a gene product, which may be a RNA molecule (gene expression) or a protein (protein expression). A gene product can be the direct transcriptional product of a gene (e.g., mRNA, tRNA, rRNA, antisense RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation.

In the present invention, the term “inserting” means to place or fit a nucleic acid sequence into the endogenous DNA. Any suitable technique for insertion of a polynucleotide into a specific sequence may be used, and several are described in the art. Suitable techniques include any method which introduces a break at the desired location and permits recombination of a vector into the gap. Thus, a crucial first step for targeted site-specific genomic modification is the creation of a double-strand DNA break (DSB) at the genomic locus to be modified. Distinct cellular repair mechanisms can be exploited to repair the DSB and to introduce the desired sequence, and these are non-homologous end joining repair (NHEJ), which is more prone to error; and homologous recombination repair (HR) mediated by a donor DNA template, that can be used to insert heterologous nucleic acid sequences. Several techniques exist to allow customized site-specific generation of DSB in the genome. Many of these involve the use of customized endonucleases, such as zinc finger nucleases (ZFNs), transcription activator-like effector nucleases (TALENs) or the clustered regularly interspaced short palindromic repeats/CRISPR associated protein (CRISPR/Cas9) system (Gaj T. et al., 2013). However, suitable techniques may also include techniques not using any DSB.

Zinc finger nucleases are artificial enzymes, which are generated by fusion of a zinc-finger DNA-binding domain to the nuclease domain of the restriction enzyme FokI. The latter has a non-specific cleavage domain, which must dimerize in order to cleave DNA. This means that two ZFN monomers are required to allow dimerization of the FokI domains and to cleave the DNA. The DNA binding domain may be designed to target any genomic sequence of interest, and may be, for example, a tandem array of Cys/His-zinc fingers, each of which recognises three contiguous nucleotides in the target sequence. The two binding sites are separated by 5-7 bp to allow optimal dimerisation of the FokI domains. The enzyme thus is able to cleave DNA at a specific site, and target specificity is increased by ensuring that two proximal DNA-binding events must occur to achieve a doublestrand break.

Transcription activator-like effector nucleases, or TALENs, are dimeric transcription factors/nucleases. They are made by fusing a TAL effector DNA-binding domain to a DNA cleavage domain (a nuclease). Transcription activator-like effectors (TALEs) can be engineered to bind practically any desired DNA sequence, so when combined with a nuclease, DNA can be cut at specific locations. TAL effectors are proteins that are secreted by Xanthomonas bacteria, the DNA binding domain of which contains a repeated highly conserved 33-34 amino acid sequence with divergent 12th and 13th amino acids. These two positions are highly variable and show a strong correlation with specific nucleotide recognition. This straightforward relationship between amino acid sequence and DNA recognition has allowed for the engineering of specific DNA-binding domains by selecting a combination of repeat segments containing appropriate residues at the two variable positions. TALENs are thus built from arrays of 33 to 35 amino acid modules, each of which targets a single nucleotide. By selecting the array of the modules, almost any sequence may be targeted. Again, the nuclease used may be FokI or a derivative thereof.

Three types of CRISPR mechanisms have been identified, of which type II is the most studied. The CRISPR/Cas9 system (type II) utilises the Cas9 nuclease to make a double-stranded break in DNA at a site determined by a short guide RNA. The CRISPR/Cas system is a prokaryotic immune system that confers resistance to foreign genetic elements. CRISPR are segments of prokaryotic DNA containing short repetitions of base sequences. Each repetition is followed by short segments of “protospacer DNA” from previous exposures to foreign genetic elements. CRISPR spacers recognize and cut the exogenous genetic elements using RNA interference.

The CRISPR immune response occurs through two steps: CRISPR-RNA (crRNA) biogenesis and crRNA-guided interference. crRNA molecules are composed of a variable sequence transcribed from the protospacer DNA and a CRISP repeat. Each crRNA molecule then hybridizes with a second RNA, known as the trans-activating CRISPR RNA (tracrRNA) and together these two eventually form a complex with the nuclease Cas9. The protospacer DNA encoded section of the crRNA directs Cas9 to cleave complementary target DNA sequences, if they are adjacent to short sequences known as protospacer adjacent motifs (PAMs). This natural system has been engineered and exploited to introduce DSB breaks in specific sites in genomic DNA, amongst many other applications. In particular, the CRISPR type II system from Streptococcus pyogenes may be used. At its simplest, the CRISPR/Cas9 system comprises two components that are delivered to the cell to provide genome editing: The Cas9 nuclease itself and a small guide RNA (sgRNA or gRNA). The gRNA is a fusion of a customised, site-specific crRNA (directed to the target sequence) and a standardised tracrRNA. Once a DSB has been made, a donor template with homology to the targeted locus is supplied; the DSB may be repaired by the homology-directed repair (HDR) pathway allowing for precise insertions to be made. Derivatives of this system are also possible. Mutant forms of Cas9 are available, such as Cas9D10A, with only nickase activity. This means, it cleaves mainly one DNA strand, and does activate NHEJ only in rare cases, dependent on the cell cycle. Instead, when provided with a homologous repair template, DNA repairs are conducted via the high-fidelity HDR pathway only. Cas9D10A (Cong et al., 2013), Cas9H840A or Cas9 N863A (Rees et al., 2019) may be used in paired Cas9 complexes designed to generate adjacent DNA nicks in conjunction with two sgRNAs complementary to the adjacent area on opposite strands of the target site, which may be particularly advantageous. The elements for making the double-strand DNA break may be introduced in one or more vectors such as plasmids for expression in the cell. Thus, any method of making specific, targeted double strand breaks in the genome in order to effect the insertion of a gene/heterologous nucleic acid sequence may be used in the method of the invention. It may be preferred that the method for inserting the gene/heterologous nucleic acid sequence utilises any one or more of ZFNs, TALENs and/or CRISPR/Cas9 systems or any derivative thereof.

Once the DSB has been made by any appropriate means, the gene/heterologous nucleic acid sequence for insertion may be supplied in any suitable fashion as described anywhere herein. The gene/heterologous nucleic acid sequence and associated genetic material form the donor DNA for repair of the DNA at the DSB are inserted using standard cellular repair machinery/pathways. How the break is initiated will alter and depends on which pathway is used to repair the damage, as noted above.

In the present invention, the term “intron” or Intervening Regions means as used throughout the whole description, a part or sequence of a gene that does not carry protein encoding information. During transcription of a gene to a pre-RNA, introns are cut (or spliced) and separated from the protein coding exons. The introns are degraded while the exons are capped and tailed to be transported out of the nucleus for further protein translation. In general, introns are much longer than exons; they can make up as much as 90% of a gene and can be over 10,000 nucleotides long. In mammals 95% of multi-exon genes undergo alternative splicing (Pan et al. 2008; Wang et al. 2008) containing introns with an average of nine introns per gene (Lander et al. 2001; Venter et al. 2001). An intron begins and ends with a specific series of nucleotides. These sequences act as the boundary between introns and exons and are known as splice sites. The recognition of the boundary between coding and non-coding DNA is crucial for the creation of functioning genes. In humans and most other vertebrate's most introns begin with 5′-GUA and end in CAG-3′ (U2-dependent intron). There are other conserved sequences found in introns of both vertebrates and invertebrates including a branch point involved in lariat (loop) formation. Further an U2-independent intron is defined through the ATATCC (5′) and YYCAC (3′) splice sites and a conserved upstream element (TCCTTAAC near the 3′-end) in these introns. Interestingly, it has been reported that RNA sequences (U12 snRNA (matches 3′ sequence) and U11 snRNA (matches 5′ sequence)) are complementary to these splicing sites and are involved in the slicing process. It may also be comprised by the present invention that an exon is not coding for a protein sequence. In protein coding genes, sometimes the 5′ or 3′-UTR (untranslated region) also contain introns. The latter leads to an instable RNA in certain conditions in coding genes because of NMD (e.g., wanted for ARC) and also 60% of non-coding RNAs have introns (Hube et al., 2015).

In the present invention, the term “gene of interest” means as used herein, a specific segment of DNA, which is desired for investigation, which may be transcribed into RNA, and which may contain an open reading frame and which encodes a protein, and also includes the DNA regulatory elements, which control expression of the transcribed region. The gene of interest may be transcribed into RNA, may contain an open reading frame and may encode a protein. In diploid organisms, a gene is composed of two alleles. It can also include an intron and the DNA regulatory elements, which control expression of the transcribed region. Thus, as used within the context of the present invention, the gene of interest comprises the intron or synthetic intron, which is used in any of the methods according to the present invention as described herein. In more detail, if the gene of interest does not contain an intron, a suitable integration point for the nucleic acid construct may be a suitable exonic region. This would create new separate exons (out of the one single exon existing before) being interrupted by a synthetic intron. This will be referred to as synthetic intron anywhere herein.

Thus, in the present invention, the term “synthetic intron” means the insertion of genetic material into a suitable exon to create a synthetic intron used in the absence of an intron within a gene of interest. This is the case in less than 10% of the eukaryotic genes.

In the present invention, the term “nucleic acid sequences” means as used throughout the whole description, a segment of DNA or RNA molecule. Here the nucleic acid sequences are defined by their function and encoding information. They are referred to as “nucleic acid construct” when more than one functionally different nucleic acid sequence is combined as mentioned above.

In the present invention, the term “nucleus” means the core of a cell in which the DNA is stored and transcribed.

In the present invention, the term “cap-independent translation” refers to the CITE (cap-independent translation element) located in the 3′-UTRs (untranslated regions) of various viruses. These sequences functionally replace the 5′-cap structure that is required for the interaction with essential translation factors (Miller et al., 2007). The term may also refer to ribosomal entry sites/internal ribosomal entry sites (IRES), which are nucleic acid elements allowing a translation initiation in a cap-independent manner.

In the present invention, the term “heterologous nucleic acid sequence” describes throughout the whole description, one or more genes suitable for the purpose that is desired for insertion into a cell. These genes may or may not be artificial or composed of functionally different compounds. It could also be defined as cargo nucleic acid or genetic sequence and may fulfil various tasks and purposes as examples are stated in the following. The genetic sequence comprised within the heterologous nucleic acid sequence may be a gene that codes a ribonucleic acid (RNA) for a protein product. Coding or messenger RNA codes for polypeptide sequences, and transcription and translation of such RNAs leads to expression of a protein within the cell. The heterologous nucleic acid sequence may in another scenario be transcribed into RNA, which functions as small nuclear RNA (snRNA), antisense RNA, microRNA (miRNA), small interfering RNA (siRNA), transfer RNA (tRNA), aptamer, design RNA (barcode RNA) and other non-coding RNAs (ncRNA), including CRISPR-RNA (crRNA) and guide RNA (gRNA).

Genetic sequences encoding the gRNAs may be included in the heterologous nucleic acid sequence. The methods of the present invention also extend to methods of knocking out endogenous genes within a cell, by virtue of the CRIPSR-Cas9 system, although any other suitable systems for gene knockout may be used. In this scenario, it is preferred that the Cas9 genes are constitutively expressed. gRNA is a short synthetic RNA composed of a scaffold sequence necessary for Cas9-binding and an approximately 20 nucleotide targeting sequence, which defines the genomic target to be modified. Thus, the genomic target of Cas9 can be changed by simply changing the targeting sequence present in the gRNA. Although the primary use of such a system is to design a gRNA to target an endogenous gene in order to knock the gene out, it can also be modified to selectively activate or repress target genes, purify specific regions of DNA, and even image DNA. All possible uses are envisaged.

Further the heterologous nucleic acid sequence may encode an enzyme, reporter or effector molecule with a function suiting the purpose and discussed somewhere else herein in detail.

Alternatively, or additionally, the heterologous nucleic acid sequence may include genes whose function requires investigation, this may include the effect of expression on the cell. The gene may include transcription factors, growth factors and/or cytokines in order for the cells to be used in cell transplantation and/or the gene may carry components of a reporter assay.

The heterologous nucleic acid sequence may include any genetic sequence, desired for transcription within the cell and the genetic sequence chosen will be dependent upon the cell type and the use to which the cell will be put after modification, as discussed somewhere else herein. Thus, the heterologous nucleic acid sequence may include a genetic sequence that is a protein-coding gene. This gene may be not naturally present in the cell, or may naturally occur in the cell, but expression of that gene is required. Alternatively, the heterologous nucleic acid sequence may be a mutated, a modified or a corrected version of a gene present in the cell, particularly for gene therapy purposes or the derivation of disease models. The heterologous nucleic acid sequence may thus include a transgene from a different organism of the same species (i.e. a diseased/mutated version of a gene from a human, or a wild-type gene from a human) or be from a different species. Examples of protein-encoding genes include, but are not limited to, the human b-globin gene, human lipoprotein lipase (LPL) gene, Rab escort protein 1 in humans encoded by the CHM gene and many more.

An heterologous nucleic acid sequence includes a desired genetic sequence, preferably a DNA sequence, that is to be transferred into a cell. The introduction of an heterologous nucleic acid sequence into the genome has the potential to alter the phenotype of that cell, either by addition of a genetic sequence that permits gene expression or knockdown/knockout of endogenous expression.

In one embodiment of the method of the present invention, the at least one nucleic acid sequence for translation of the nucleic acid construct or part thereof is a nucleic acid sequence for translation of the heterologous nucleic acid sequence.

In a further embodiment of the method of the present invention, the nucleic acid construct or part thereof is under the control of an endogenous promoter of the gene comprising the expression product of the nucleic acid construct or part thereof.

In the present invention, the term “endogenous” means with an internal cause of origin and refers here to the cell selected for the application of the invented method disclosed herein. The term specifically comprises the genetic material and metabolite of said selected cell, which occur naturally and are necessary for that particular cell.

In the present invention, the term “endogenous promotor” means a nucleic acid sequence with internal cause of origin regulating and supporting the gene expression in the cell selected for the application of the invented method disclosed herein.

In a further embodiment of the method of the present invention, the at least one nucleic acid sequence for transcription of the nucleic acid construct or part thereof comprises a splice donor nucleic acid sequence and a splice acceptor nucleic acid sequence. Preferably, the splice donor nucleic acid sequence comprises or consists of a sequence being at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% identical or homologue to the SEQ ID NO: 1 as depicted herein. Preferably, the splice acceptor nucleic acid sequence comprises or consists of a sequence being at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% identical or homolog to the SEQ ID NO: 1 as depicted herein. More preferably, the splice donor nucleic acid sequence comprises or consists of SEQ ID NO: 1 and/or the splice acceptor nucleic acid sequence comprises or consists of SEQ ID NO: 2 (or a sequence which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 2).

The term “homology” (or being “homologue”) is used herein in its usual meaning and includes identical amino acids as well as amino acids, which are regarded to be conservative substitutions (for example, exchange of a glutamate residue by an aspartate residue) at equivalent positions in the linear amino acid sequence of two proteins that are compared with each other. By “identity” or “sequence identity” (or being “identical”) is meant a property of sequences that measures their similarity or relationship.

In one embodiment of the present invention, the nucleic acid construct also comprises at least one nucleic acid sequence for excision of the nucleic acid construct or part thereof out of the intron or synthetic intron. In the present invention, the term “nucleic acid sequences for excision” refers to a nucleic acid sequence as defined somewhere else herein, which is recognizable and can be cut. The so-called splice donor and splice acceptor sequence enable the scaled removal of the nucleic acid construct from the intron or synthetic intron of the cell selected for the method of the present invention as described herein. Further, the genetic material may be provided together with other cleavable sequences. Such sequences are sequences that are recognized by an entity capable of specifically cutting DNA, and include restriction sites, which are the target sequences for restriction enzymes or sequences for recognition by other DNA cleaving entities, such as nucleases, recombinases, ribozymes or artificial constructs. At least one cleavable sequence may be included, but preferably two or more are present.

In the present invention, the term “splice donor” means a nucleic acid sequence controlling the splicing process by being recognizable to the spliceosome as cutting site. After the cutting process the remaining exons can be re-ligated together.

In the present invention, the term “splice acceptor” means a nucleic acid sequence controlling the splicing process by being recognizable to the spliceosome as cutting site. After the cutting process the remaining exons can be re-ligated together.

In a further embodiment of the method of the present invention, the at least one nucleic acid sequence for exporting the nucleic acid construct or part thereof out of the nucleus is a viral sequence. Preferably, the respective viral sequence comprises or consists of a sequence being at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% identical or homologue to the SEQ ID NO: 3 or SEQ ID NO: 25 as depicted herein. Preferably, on a further embodiment, the respective viral sequence comprises or consists of a sequence being at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% identical or homologue to the SEQ ID NOs: 4 or 42 as depicted herein. More preferably, the viral sequence comprises or consists of CTE according to SEQ ID NO: 3 or SEQ ID NO: 25 and/or comprises or consists of WPRE according to SEQ ID NOs: 4 or 42.

In the present invention, the term “viral sequence” means a nucleic acid sequence being of a viral origin. Such a sequence is used to stimulate a nuclear export of the nucleic acid construct. Here, preferably, CTE (constitutive transport element) of type D viruses are cis-activating elements that promote nuclear export of incompletely spliced mRNAs and WRPE (woodchuck hepatitis post-transcriptional regulatory element), which increases the expression, are used.

In the present invention, the term “CTE” means constitutive transport element, a viral cis-activating element that promotes nuclear export. However, other RTE (RNA transport elements) may be suitable too like CTE, e.g. IAP or RTE or its mutant (RTEm26).

As used within the present invention, the term “WPRE” means woodchuck hepatitis post-transcriptional regulatory element, which is a viral sequence used to increase the expression of a transcript.

In one embodiment of the method of the present invention, the at least one nucleic acid sequence for translation of the nucleic acid construct or part thereof is for translation of the heterologous nucleic acid sequence and is initiated by an internal ribosomal entry site (IRES) and an open reading frame (ORF). Preferably, the internal ribosomal entry site (IRES) comprises or consists of a sequence being at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% identical or homologue to the SEQ ID NO: 5 as depicted herein. Preferably, in a further embodiment, the internal ribosomal entry site (IRES) comprises or consists of a sequence being at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% identical or homologue to the SEQ ID NO: 6 as depicted herein. More preferably, the internal ribosomal entry site (IRES) is the internal ribosomal entry site of the virus Encephalomyocarditis virus (EMCV) according to SEQ ID NO: 5 or the internal ribosomal entry site of the Hepatitis C virus (HCV) according to SEQ ID NO: 6. In a further embodiment of the method of the present invention, at least one heterologous nucleic acid sequence enables cap-independent translation, preferably via an internal ribosomal entry site (IRES), more preferably via an internal ribosomal entry site (IRES) from a virus such as the Encephalomyocarditis virus (EMCV) or the Hepatitis C virus (HCV); and an open reading frame.

As used within the present invention, the term “internal ribosomal entry site (IRES)” means a nucleic acid sequence of viral origin that recruits ribosomes and allows end-independent translation. Viruses containing internal ribosomal entry sites are, as already mentioned, the Encephalomyocarditis virus (EMCV) and the Hepatitis C virus (HCV) which are preferred IRES donors.

In the present invention, the term “open reading frame” describes the stretch of nucleotide region ranging from initiation codon to stop codon, which is translated into protein. It is defined by the tRNA triplet system, each coding for a certain amino acid. A shift in this coding triplet system or reading frame can change the resulting amino acid and thus the polypeptide chain of a protein. The open reading frame as used herein includes a start and a stop codon enabling the protein translation.

In one embodiment of the method of the present invention, the at least one nucleic acid sequence for preventing degradation of the nucleic acid construct or part thereof is a poly-A-tail. Preferably, the poly-A-tail is a synthetic poly-A-tail. More preferably, the synthetic poly-A-tail comprises at least 30 adenosines. Even more preferred, the poly A-tail used in the present invention is depicted in SEQ ID NO: 7 (or a sequence which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 7).

In the present invention, the term “synthetic poly-A-tail” means multiple adenosine monophosphates synthetically liked together or of synthetic or exogenous origin.

In a further embodiment of the method of the present invention, the at least one nucleic acid sequence for preventing degradation of the nucleic acid construct or part thereof is a polyadenylation signal. Preferably, the polyadenylation signal is a late SV40 polyadenylation signal and a rabbit beta-globin polyadenylation signal. More preferably, the late SV40 polyadenylation signal is mutated to be unidirectional. It is also preferred that the polyadenylation signals are integrated in the nucleic acid construct in an antisense direction and that they are enclosed with loxP sites and that after transcription, the inverted polyadenylation signal is not separated from the endogenous gene product. It is even more preferred that after transcription a Cre recombinase is administered to the transcript to invert the polyadenylation signals into sense direction. Even more preferred, the Cre recombinase as used within the present invention is depicted herein in SEQ ID NO: 8 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 8, e.g., having Cre recombinase activity).

As used within the present invention, the term “polyadenylation signals of late SV40” is a certain mammalian terminator sequence that signals the end of a transcriptional unit. It is originated from the Simian-Virus 40. Polyadenylation signals are in the method of this invention integrated in a way that they can be inverted via Cre-recombinase via loxP sites and lead to a premature termination of the transcription. The knock-out event can thus be monitored by deactivation of the downstream intron-encoded reporter.

In the present invention, the term “rabbit beta-globin polyadenylation signal” means a certain mammalian terminator sequence that signals the end of a transcriptional unit. It is originated from the rabbit beta-globin gene. Polyadenylation signals are in the method of this invention integrated in a way that they can be inverted via Cre-recombinase via loxP sites and lead to a premature termination of the transcription. The knock-out event can thus be monitored by deactivation of the downstream intron-encoded reporter. This is also described by the term “FLExing” which comprises a flanked DNA part with semi-orthogonal loxP sites. Here, “semi-orthogonal” means that both loxP sites are recognized by Cre recombinase, but the different loxP sites are not compatible.

As used within the present invention, the term “Cre-recombinase” means Type I topoisomerase recognizing DNA loxP sites and is able to excise, fuse and inverse the DNA fragment within the loxP sites. In one scenario of the present invention, the polyadenylation signal is integrated into antisense direction (i.e. inverted) and enclosed by loxP sites. The inverted poly A-signal is not separated from the endogenous gene product throughout transcription, but can be switched into sense direction by adding the Cre recombinase. This enzyme is cutting and thus turning the reading direction of the poly A-signal, which is then re-ligated to the endogenous gene product. This enables an induced premature polyadenylation, leading to a degradation of the endogenous gene product and further leading to a silencing of the very same. To further ensure this system, it may be preferred to add an additional splice acceptor to this system. It may be placed at the 3′ end next to the loxP site of the inverted poly A-tail. This splice acceptor is directed into anti-sense direction to be switched into sense direction together with the poly A-tail. Thus, after the transcription and the Cre-recombinase-induced switch of the polyadenylation signal into sense direction, the splice acceptor is likewise switched into sense direction and thus leading to the loss of a small piece of the poly A-tail further ensuring the premature polyadenylation and later degradation of this genetic combination.

In the present invention, the term “loxP sites” means a cleavable genetic sequence recognized by enzymes such as Cre recombinase. It allows direct replacement of the removed insertion. Alternatively or additionally, the cleavable site may be the rox site for Cre recombinase. The nucleic acid construct may also include other cleavable sequences. Such sequences are sequences that are recognized by an entity capable of specifically cutting DNA, and include restriction sites, which are the target sequences for restriction enzymes or sequences for recognition by other DNA cleaving entities, such as nucleases, recombinases, ribozymes or artificial constructs. At least one cleavable sequence may be included, but preferably two or more are present.

In one further embodiment of the method of the present invention, the method is non- or minimally invasive for the expression product of the intron or synthetic intron, such that a native and/or fully functional protein is expressed compared to the protein without insertion of the nucleic acid construct or part thereof.

In the present invention, the term “non- or minimally invasive” means a non-destructive method that enables a scarless excision of the nucleic acid construct wherein the mature mRNA of the endogenous gene is not modified. It refers to the gene product of an endogenous gene selected for use in the method of the present invention being indistinguishable from the same endogenous gene of interest not treated with the method of the present invention. This scarless excision can be established by integrating a splice donor and a splice acceptor, two sequences separating the integrated coding sequence from the endogenous coding sequence.

In a further embodiment of the method of the present invention, the insertion of the nucleic acid construct is with targeted transgene insertion. The term “targeted transgene insertion”, as used within the present invention, has the common meaning being known by a person skilled in the art. Traditionally, transgene insertion is targeted to a specific locus by provision of a plasmid carrying a transgene, and containing substantial DNA sequence identity flanking the desired site of integration. Spontaneous breakage of the chromosome followed by repair using the homologous region of the plasmid DNA as a template results in the transfer of the intervening transgene into the genome. The term “sequence” refers to a nucleotide sequence of any length, which can be DNA or RNA. Further it can be linear, circular or branched, and either single-stranded or double stranded. The term “transgene” refers to a nucleotide sequence that is inserted into a genome. A transgene can be of any length, for example between 2 and 100,000,000 nucleotides in length (or any integer value therebetween or thereabove), preferably between about 100 and 100,000 nucleotides in length (or any integer therebetween), more preferably between about 2000 and 60,000 nucleotides in length (or any value therebetween) and even more preferable, between about 3 and 15 kb (or any value therebetween).

In one embodiment of the method of the present invention, the at least one heterologous nucleic acid sequence encodes for a protein-coding RNA, a non-coding RNA, a miRNA, an aptamer, a siRNA, a synthetic RNA sequence or a barcode for extranuclear detection. In a further embodiment of the method of the present invention, the at least one heterologous nucleic acid sequence is detected and enables to detect a specific cell. When using the method to export a transcript that is not coding for a gene, such as a RNA-barcode that can be secreted by the cellular-export unit based on gag, it is preferred that such a non-coding RNA may also be a guide RNA for CRISPR effectors such as Cas13, which act in the nucleus (with lower priority also Cas9 variants although they have to act in the nucleus). More generally, the described method can export an intron-encoded transcript into the cytosol, which can then be translated into an effector protein or can be used as an RNA-barcode for sequence-based analysis of cell states either in the cytosol or after secretion from the cell or the transcript can also be an effector molecule itself that can influence cellular processes, for instance as guide RAN for Cas13.

In one embodiment of the method of the present invention, the at least one heterologous nucleic acid sequence is detected and provides information about the transcriptional regulation of the cell or a time stamp that is a time resolved information about a cellular process.

In another embodiment of the method of the present invention, the at least one heterologous nucleic acid sequence encodes for a protein-coding RNA, non-coding RNA, miRNA, aptamer, siRNA, or a designed RNA sequence that encodes the identity of the modified cells (commonly referred to as a barcode) and/or further provides information about the transcriptional regulation of the cell or a time stamp of a cellular process.

As used within the present invention, the term “non-coding RNA” means an RNA molecule not carrying the information to build a protein. The desired nucleic acid sequence for insertion is preferably a DNA sequence that encodes an RNA molecule. The RNA molecule may be of any sequence, but is preferably a non-coding RNA. A non-coding RNA may be functional and may include without limitation: microRNA, small interfering RNA, piwi-interacting RNA, antisense RNA, small nuclear RNA, small nucleolar RNA, Small Cajal Body RNA, Y RNA, Enhancer RNAs, Guide RNA, Ribozymes, Small hairpin RNA, Small temporal RNA, Trans-acting RNA, small interfering RNA and subgenomic messenger RNA. Non-coding RNA may also be known as functional RNA. Several types of RNA are regulatory in nature, and, for example, can downregulate gene expression by being complementary to a part of an mRNA or a gene's DNA. microRNAs (miRNA; usually 21-22 nucleotides) are found in eukaryotes and act through RNA interference (RNAi), where an effector complex of miRNA and enzymes can cleave complementary mRNA, block the mRNA from being translated, or accelerate its degradation. Another type of RNA, small interfering RNAs (siRNA; usually 20-25 nucleotides long) act through RNA interference in a fashion similar to miRNAs. Some miRNAs and siRNAs can cause genes they target to be methylated, thereby decreasing or increasing transcription of those genes. Animals have Piwi-interacting RNAs (piRNA; usually 29-30 nucleotides long) that are active in germline cells and are thought to be a defence against transposons. Many prokaryotes have CRISPR RNAs, a regulatory system similar to RNA interference, and such a system include guide RNA (gRNA). Antisense RNAs are widespread, most downregulate a gene but a few are activators of transcription. Antisense RNA can act by binding to an mRNA, forming double-stranded RNA that is enzymatically degraded. There are many long non-coding RNAs that regulate genes in eukaryotes, one such RNA is Xist, which coats one X chromosome in female mammals and inactivates it. Thus, there are a multitude of functional RNAs some of which are described above that can be employed in the any of the methods of the present invention.

Further, the heterologous nucleic acid sequence may encode non-coding RNA, whose function is to knockdown the expression of an endogenous gene or DNA sequence encoding non-coding RNA in the cell. Alternatively, the genetic sequence may encode guide RNA for the CRISPR-Cas9 system to effect endogenous gene knockout. The methods of the invention thus also extend to methods of knocking down endogenous gene expression within a cell. The non-coding RNA may suppress gene expression by any suitable means including RNA interference and antisense RNA. Thus, the genetic sequence may encode a shRNA, which can interfere with the messenger RNA for the endogenous gene. The reduction in endogenous gene expression may be partial or full—i.e. expression may be at least 50, 55, 60, 65, 70, 75, 80, 85, 90, 91, 92, 93, 94, 95, 96, 97, 98, 99 or 100% reduced compared to the cell prior to induction of the transcription of the non-coding RNA.

As used within the present invention, the term “aptamer” means short single-stranded DNA- or RNA-based oligonucleotides that can selectively bind to small molecular ligands or protein targets with high affinity and specificity, when folded into their unique three-dimensional structures.

In the present invention, the term “siRNA” means small interfering Ribonucleic Acid also known as short interfering RNA or silencing RNA and describes a double-stranded RNA molecule as discussed somewhere else herein.

As used within the present invention, the term “RNA barcode” means a non-coding RNA that is synthesised with a recognizable sequence and thus enables to identify a cell or gene transfected with this RNA information. The term “barcode” or “bar-code” as used within the present invention may be a detectable representation of data containing information about the object the bar-code is associated with. In accordance with the present invention, the bar-code may be a pre-determined, i.e. known, nucleic acid sequence consisting of nucleotides in a particular order. In the present invention, the term “barcode” may also mean a synthesised nucleic acid of precisely known sequence and length, which may be linked to a gene sequence of interest through a linker sequence. This synthesised nucleic acid sequence enables a read-out of endogenous gene transcripts by decoding the before defined barcode. It therefore is a type of reporter sequence enabling e.g. to count the frequency of a gene being transcribed.

As used within the present invention, the term “time stamp” describes a special use of a RNA sequence or barcode as defined above. Here, the synthetic sequence is expressed in a time dependent manner and may result e.g. in a combination of transcription frequency through the barcode itself and time resolved information through inducible promotors.

In one embodiment of the method of the present invention, the heterologous nucleic acid sequence encodes a protein or enzyme selected from the group consisting of a fluorescent protein, preferably green fluorescent protein; a bioluminescence-generating enzyme, preferably NanoLuc, NanoKAZ, TurboLuc, Cypridina, Firefly, Renilla luciferase, split luciferase, split APEX2 or mutant derivatives thereof; an enzyme, which is capable of generating a coloured pigment, preferably tyrosinase or an enzyme of a multi-enzymatic process, more preferably the violacein or betanidin synthesis process, a genetically encoded receptor for multimodal contrast agents, preferably Avidin, Streptavidin or HaloTag or mutant derivatives thereof; an enzyme, which is capable of converting a non-reporter molecule into a reporter molecule, preferably TEV protease and picornaviral proteases, more preferably rhinoviral 3C proteases and polioviral 3C protease, SUMO proteases and mutant derivatives thereof; an enzyme, which is capable of inactivating a toxic compound, preferably blasticidin-S-deaminase, puromycin-N-acetyltransferase, neomycin phosphotransferase, hygromycin B phosphotransferase and mutant derivatives thereof, an enzyme, which is capable of converting pro-drug/toxin-mediated toxicity, preferably thymidine kinase and mutant derivatives thereof and a small-molecule sensor protein, preferably calmodulin, troponin C, S100 and mutant derivatives thereof.

As mentioned somewhere else herein, the heterologous nucleic acid sequence as used herein may relate to a gene, which encodes a protein that is not (naturally) present in a cell. Such material includes genes for markers or reporter molecules, such as genes that induce visually identifiable characteristics including fluorescent and luminescent proteins. Examples include the gene that encodes jellyfish green fluorescent protein (GFP), which causes cells that express it to glow green under blue/UV light, luciferase, which catalyses a reaction with luciferin to produce light, and the red fluorescent protein from the gene dsRed. As outlined herein, the expression product of the heterologous nucleic acid sequence or part thereof may be used to detect cells, in which the nucleic acid construct was inserted. This is possible, because the detection of the expression product of the heterologous nucleic acid sequence or part thereof marks cells, in which the respective genetic sequence has been inserted. Thus, those cells can be selected or isolated.

Such markers or reporter genes are useful, since the presence of the reporter protein confirms gene or protein expression, indicating successful insertion of the construct. Selectable markers may further include resistance genes to antibiotics or other drugs. Markers or reporter gene sequences can also be introduced that enable studying the expression of endogenous (or exogenous genes). This includes Cas proteins, including CasL, Cas9 proteins that enable excision of genes of interest, as well as Cas-fusion proteins that mediate changes in the expression of other genes, e.g. by acting as transcriptional enhancers or repressors. Moreover, non-inducible expression of molecular tools may be desirable, including optogenetic tools, nuclear receptor fusion proteins, such as tamoxifen-inducible systems ERT, and designer receptors exclusively activated by designer drugs. Furthermore, sequences that code signalling factors that alter the function of the same cell or of neighbouring or even distant cells in an organism, including hormones autocrine or paracrine factors, which may be co-expressed with the same promotor as the transcriptional regulator protein. Additionally, the further genetic material may include sequences coding for non-coding RNA, as discussed herein. Examples of such genetic material includes genes for miRNA, which may function as a genetic switch.

In a further embodiment of the method of the present invention, the method further comprises combining the expression of the protein or enzyme encoded by the heterologous nucleic acid sequence to the natural expression of the gene comprising the nucleic acid construct or part thereof by using the same promotor.

In one further embodiment of the method of the present invention, the heterologous nucleic acid sequence encodes a resistance gene for cell-toxic compounds. Preferably, the method additionally comprises detecting the survival of the cells comprising the nucleic acid construct or part thereof. More preferably, the resistance gene for cell-toxic compounds is used as a selection marker of the cells comprising the nucleic acid construct or part thereof.

In one embodiment of the method of the present invention, the heterologous nucleic acid sequence encodes a Cas (i.e., CRISPR-associated) enzyme, e.g., selected from the group consisting of: Cas9 (e.g., CRISPR-associated endonuclease Cas9, e.g., having EC:3.1.-.- enzymatic activity and/or SEQ ID NO: 9 or UniProtKB Accession Number/s: Q99ZW2, G3ECR, J7RUA5, A0Q5Y3, J3F2B0, C9X1G5, Q927P4, Q8DTE3, Q6NKI3, A11Q68 or Q9CLT2);

Cas12a (e.g., CRISPR-associated endonuclease Cas12a, e.g., having EC:3.1.21.1 and/or EC:4.6.1.22 enzymatic activity and/or UniProtKB Accession Number/s: A0Q7Q2, A0A182DWE3 or U2UMQ6, e.g., U2UMQ6 enzyme and/or its variants/mutants may also referred to as Cas12a/Cpf1 enzymes and/or is/are the preferred Cas12a enzyme/s for use in mammalian systems);
Cas12b (e.g., CRISPR-associated endonuclease Cas12b, e.g., having EC:3.1.-.- enzymatic activity and/or UniProtKB Accession Number/s: T0D7A2, e.g., T0D7A2 enzyme and/or its variants/mutants may have temperature optimum at about 48° C. and/or may be the preferred Cas12b enzyme/s for use in non-mammalian systems and/or in organisms able to function at a temperature at about 48° C. and/or about 37° C. (e.g., BhCas12b, e.g., having RefSeq Accession Number: WP_095142515.1 and/or BhCas12b v4 mutant/s comprising: K846R and/or S893R and/or E837G mutations, e.g., using the numbering of WP_095142515.1; e.g., as reported by Strecker et al., 2019; Nat Commun. 2019 Jan. 22; 10(1):212. doi: 10.1038/s41467-018-08224-4));
Cas12c (e.g., CRISPR-associated protein 12c, e.g., selected from the group consisting of: SEQ ID NO: 34 (Cas12c1), SEQ ID NO: 35 (Cas12c2) and SEQ ID NO: 36 (OspCas12c); e.g., as reported by Yan et al., 2019; Science. 2019 Jan. 4; 363(6422):88-91. doi: 10.1126/science.aav7271. Epub 2018 Dec. 6;
Cas13a (e.g., CRISPR-associated endoribonuclease Cas13a, e.g., having EC:3.1.-.- enzymatic activity and/or UniProtKB Accession Number/s: C7NBY4, P0DOC6, U2PSH1, A0A0H5SJ89, P0DPB7, E4T0I2 or P0DPB8);
Cas13b (e.g., CRISPR-associated protein 13b, e.g., UniProtKB Accession Number/s: E6K398)
Cas13d (e.g., CRISPR-associated protein 13d, e.g., UniProtKB Accession Number/s: B0MS50 or A0A1C5SD84);
Cas14 (e.g., CRISPR-associated protein Cas14, e.g., GenBank Accession Number/s: QBM02559.1, SUY72868.1, VEJ66719.1, SUY81478.1, SUY85836.1 or STC69301.1);

### CasX (e.g., UniProtKB Accession Number/s: A0A357BT59);

and/or sequences which are at least 60% or more, e.g., at least 65%, at least 70%, at least 75%, at least 80%, at least 85%, at least 90%, at least 95%, at least 96%, at least 97%, at least 98%, at least 99% or 100% identical to sequences as described herein (e.g., having the corresponding Cas enzymatic activity) and/or fusion proteins thereof. The Cas9 enzymes of the present invention may preferably refer to the sequence according to SEQ ID NO: 9 as depicted herein.

In a further embodiment of the method of the present invention, the heterologous nucleic acid sequence encodes an amino acid, which can be metabolized to an antibiotic or derivative thereof or which can be a part or play a role of/in an antibiotic synthesis, preferably for inducing a genetic system, more preferably for inducing the genetic Tet-On/Tet-OFF system.

In the present invention, the term “antibiotic” means a synthetic or natural agent used to fight or destroy bacteria. Here an antibiotic of the Tetracycline family or a deviate thereof is preferred. As used within the present invention, the term “Tet-On/Tet-OFF system” means a genetic function of bacterial origin, which links the expression to the addition of antibiotics, such as tetracycline or a derivate thereof. Tet-On means that the tetracycline operator is blocked by the tetracycline repressor until tetracycline is added. The repressor binds to tetracycline such that the operator is free and transcription can start. Tet-OFF means that in the presence of tetracycline, the expression from a tet-inducible promoter is reduced.

In a further embodiment of the method of the present invention, the heterologous nucleic acid sequence encodes an enzyme of a biosynthesis pathway generating a toxin or a mutant thereof. An example of such an enzyme may be the N-acetylhydrolase derived from Streptomyces alboniger hydrolysing N-acetylpuromycin to puromycin. In this context, a toxin may be a protein synthesis inhibitor, very well known to the person skilled in the art, such as puromycin, tetracyclin (e.g., can be used against bacteria), blasticidin S, chloroamphenicol (e.g., can be used against bacteria and/or mammalian cells in suitable concentrations) or neomycin or chemical isoforms thereof.

In one embodiment of the method of the present invention, the heterologous nucleic acid sequence is a suicide gene or a gene, which induces a cell death cascade.

In the present invention, the term “suicide gene” is also called prodrug transforming gene and describes genes encoding enzymes, which can transform the non-toxic prodrug substrate into toxic drugs. Further suicide genes are genes that express a protein that causes the cell to undergo apoptosis, or alternatively may require an externally supplied co-factor or co-drug in order to work. The co-factor or co-drug may be converted by the product of the suicide gene into a highly cytotoxic entity. For example, the non-toxic 5F-cytosine (5Fc) can be transformed into cancer toxic 5F-uracil (5Fu) by the CD from Escherichia coli and the nontoxic ganciclovir (GCV) can be transformed into cancer toxic phosphorylated GCV (P-GCV) by the HSV deoxythymidine kinase (TK). Because the prodrugs are non-toxic and are transformed by these genes to form a toxic drug to kill themselves, these genes are called suicide genes. In some circumstances, it may be desirable to include a suicide gene in the heterologous nucleic acid sequence, should the genetic sequence itself not be a suicide gene for cancer gene therapy. The suicide gene may use the same inducible promoter within the heterologous nucleic acid sequence, or it may be a separate inducible promoter to allow for separate control. Such a gene may be useful in gene therapy scenarios, where it is desirable to be able to destroy donor/transfected cells if certain conditions are met. Chemotherapeutic suicide gene therapy approaches are known as gene-directed enzyme prodrug therapy. Suicide gene therapy approaches using deactivated drugs are known as gene-directed enzyme prodrug therapy (GDEPT) or gene-prodrug activation therapy (GPAT).

Further, a non-limiting example of a protein inducing the cell death cascade might be p53, a protein usually activated through DNA damage in healthy cells capable of inducing apoptosis to the very same cell.

The protein sequence of i53 is depicted herein in SEQ ID NO: 11.

In a further embodiment of the method of the present invention, the heterologous nucleic acid sequence further comprises a polynucleotide encoding a protein, which functions as an activator of the expression of the gene comprising the nucleic acid construct or part thereof.

As used within the present invention, the term “activator of the expression” means a small RNA or transcription factor introducing or supporting the gene expression. Alternatively, should the cell be a stem cell, the heterologous nucleic acid sequence may include as genetic sequence encoding a key lineage specific master regulator, abbreviated here are master regulator. Master regulators may be one or more of: transcription factors, transcriptional regulators, cytokine receptors or signalling molecules and the like. A master regulator is an expressed gene that influences the lineage of the cell expressing it. It may be that a network of master regulators is required for the lineage of a cell to be determined. As used herein, a master regulator gene that is expressed at the inception of a developmental lineage or cell type, participates in the specification of that lineage by regulating multiple downstream genes either directly or through a cascade of gene expression changes. If the master regulator is expressed, it has the ability to re-specify the fate of cells destined to form other lineages.

In one embodiment of the method of the present invention, the heterologous nucleic acid sequence encodes a transcription factor. Preferably, the transcription factor is used to force or refine determination of a stem cell into a defined mature cell.

As used within the present invention, the term “transcription factor” means master regulator proteins possessing domains that bind to the DNA of promoter or enhancer regions of specific genes and functionally support or enable the gene to be expressed. They also possess a domain that interacts with RNA polymerase II or other transcription factors and consequently regulates the amount of messenger RNA (mRNA) produced by the gene.

Alternatively, the heterologous nucleic acid sequence may express growth factors, including BDNF, GDF, NGF, IGF, FGF and/or enzymes that can cleave pro-peptides to form active forms. Gene therapy may also be achieved by expression of a genetic sequence including a genetic sequence encoding an antisense RNA, a miRNA, a siRNA or any type of RNA that interferes with the expression of another gene within the cell.

In a further embodiment of the method of the present invention, the transcription factor is used to force or refine determination of a stem cell into a defined mature cell which is also discussed somewhere else herein.

In the present invention, the term “stem cell” means an elementary type of cell that has the potential to divide or to produce more cells, or to develop into any cell that has a particular character. In this invention the used stem cells might be pluripotent stem cell. The heterologous nucleic acid sequence could be used to refine the reprogramming and differentiation of stem cells. Where the aim is to produce mature cell types from progenitor cells, the cell, which is modified, is a stem cell, preferably a pluripotent stem cell. Pluripotent stem cells have the potential to differentiate into almost any cell in the body. There are several sources of pluripotent stem cells. Embryonic stem cells (ES cells) are pluripotent stem cells derived from the inner cell mass of a blastocyst, an early-stage pre-implantation embryo. Induced pluripotent stem cells (iPSCs) are adult cells that have been genetically reprogrammed to an embryonic stem cell-like state by being forced to express genes and factors important for maintaining the defining properties of embryonic stem cells. In 2006 it was shown that the introduction of four specific genes encoding transcription factors could convert adult cells into pluripotent stem cells (Takahashi, K; Yamanaka, S (2006), Cell 126 (4): 663-76), but subsequent work has reduced/altered the number of genes that are required. Oct-3/4 and certain members of the Sox gene family have been identified as potentially crucial transcriptional regulators involved in the induction process. Additional genes including certain members of the Klf family, the Myc family, Nanog, and LIN28, may increase the induction efficiency. Examples of the genes, which may be contained in the reprogramming factors include Oct3/4, Sox2, SoxI, Sox3, SoxI5, SoxI7, Klf4, Klf2, c-Myc, N-Myc, L-Myc, Nanog, Lin28, FbxI5, ERas, ECAT15-2, Tell, beta-catenin, Lin28b, SalII, SalI4, Esrrb, Nr5a2, Tbx3 and GlisI, and these reprogramming factors may be used singly, or in combination of two or more kinds thereof.

Where the aim is to produce stem cells with a gene knockdown or knock out for further research, such as developmental or gene function studies, the cell, which is modified may be a stem cell, preferably a pluripotent stem cell, or a mature cell type. Sources of pluripotent stem cells are discussed elsewhere. If the cells modified by insertion of an heterologous nucleic acid sequence are to be used in a human patient, it may be preferred that the cell is an iPSC derived from that individual. Such use of autologous cells would remove the need for matching cells to a recipient. Alternatively, commercially available iPSC may be used, such as those available from WiCell® (WiCell Research Institute, Inc, Wisconsin, US).

In a further embodiment of the method of the present invention, the heterologous nucleic acid sequence encodes a transcriptional regulator or a repressor protein or an intrabody.

As used within the present invention, the term “transcriptional regulator” sums up transcription factors, co-factors, chromatin remodelers and all factors influencing the DNA to RNA transcription.

In the present invention, the term “repressor protein” describes a protein, in which its binding to the operator inhibits the transcription of one or more genes.

In one additional embodiment of the method of the present invention, the heterologous nucleic acid sequence encodes a protein, which is a hormone or has the function of a hormone.

As used within the present invention, the term “hormone” means a regulatory substance produced in an organism or cell and is transported in tissue by fluids, such as blood to stimulate specific cells or tissues into action.

In a further embodiment of the method of the present invention, the heterologous nucleic acid sequence encodes a protein, which is a receptor, preferably a hormone receptor or a mutant derivate thereof.

As used within the present invention, the term “hormone receptor” describes a subset of a huge number of molecules that are utilized by all cells to receive specific information from other cells and the external environment.

In one embodiment of the method of the present invention, the heterologous nucleic acid sequence encodes an affinity domain or tag to bind protein, DNA or RNA. Preferably, the protein affinity domain is used to capture the expression product of the nucleic acid construct or part thereof, more preferably the expression product of the heterologous nucleic acid sequence.

In the present invention, the term “affinity domain” means a protein or protein part with a high degree and tendency to bind to certain other substances, proteins or parts thereof.

As used within the present invention, the term “tag” includes a peptide, amino acid, protein or nucleic acid that is able to bind to other substances and thus can improve solubility, detection, purification, localization, identification or expression of that substance. A tag usually binds substances with an affinity domain as defined somewhere else herein.

In a further embodiment of the method of the present invention, the heterologous nucleic acid sequence encodes an antibody or antibody fragment. Preferably, the antibody or antibody fragment is used to capture the expression product of the nucleic acid construct or part thereof, preferably the expression product of the heterologous nucleic acid sequence.

As used within the present invention, the term “antibody” means a protein produced by the immune system in response to, and counteracting a specific antigen. Antibodies bind chemically to substances, which the body recognizes as alien, such as bacteria, viruses, and foreign substances in the blood.

In one embodiment of the method of the present invention, the protein or enzyme encoded by the heterologous nucleic acid sequence is for preventing pathological changes within the cell.

In one embodiment of the method of the present invention, the method is for detecting biological functions, preferably the regulation of tissue and cell generation, more preferably neuro-regeneration.

In the present invention, the term “tissue generation” or “tissue engineering” means to rebuild specialized cells with the purpose of renewing or replacing cells, tissues or even whole organs of a human or animal. Methods of tissue engineering are known to those skilled in the art, but include the use of a scaffold (an extracellular matrix) upon which the cells are applied in order to generate tissues/organs. These methods can be used to generate an “artificial” windpipe, bladder, liver, pancreas, stomach, intestines, blood vessels, heart tissue, bone, bone marrow, mucosal tissue, nerves, muscle, skin, kidneys or any other tissue or organ. Methods of generating tissues may include additive manufacturing, otherwise known as three-dimensional (3D) printing, which can involve directly printing cells to make tissues.

As used within the present invention, the term “cell generation” means the reprogramming of pluripotent stem cells into mature cells. In this aspect of the present invention, the heterologous nucleic acid sequence for insertion into the intron consists of preferably one or more master regulators. These heterologous nucleic acid sequences may enable the cell to be programmed into a particular lineage, and different heterologous nucleic acid sequences will be used in order to direct differentiation into mature cell types. Any type of mature cell is contemplated.

Where the cell used in any of the methods of the present invention is pluripotent, the resultant cell may be a lineage restricted-specific stem cell, progenitor cell or a mature cell type with the desired properties, by expression of a master regulator. These lineage-specific stem cells, progenitor or mature cells may be used in any suitable fashion. For example, the mature cells may be used directly for transplantation into a human or animal body, as appropriate for the cell type. Alternatively, the cells may form a test material for research, including the effects of drugs on gene expression and the interaction of drugs with a particular gene. The cells for research can involve the use of an heterologous nucleic acid sequence with a genetic sequence of unknown function, in order to study the controllable expression of that genetic sequence. Additionally, it may enable the cells to be used to produce large quantities of desirable materials, such as growth factors or cytokines.

As used within the present invention, the term “neuroregeneration” means the growth or repair of nervous tissue or cells. This may include renewed neurons, glia cells, axons, myelin sheets or synapses.

In one embodiment of the method of the present invention, the method is for detecting intrabodies, e.g. encoded by INSPECT. This is to be understood to be opposed to the other options for an INSPECT encoded reporter as mentioned herein, such as luciferase or fluorescent proteins. With this embodiment, the skilled person would have the additional benefit that the stoichiometries of intrabody to target can be controlled, because intrabodies are only expressed if the target is expressed, resulting in a 1:1 stoichiometry.

The present invention also relates to a nucleic acid construct or part thereof comprising or consisting of any of SEQ ID NOs: 1 to 43 (and sequences which are at least 60% or more, e.g., at least 65%, at least 70%, at least 75%, at least 80%, at least 85%, at least 90%, at least 95%, at least 96%, at least 97%, at least 98%, at least 99% or 100% identical to sequences having SEQ ID NOs: 1 to 43 as described herein). It is preferred that such a nucleic acid construct or part thereof is for use in therapy. It is also preferred that such a nucleic acid construct or part thereof is for use in the treatment or prevention of cancer.

As used within the present invention, the term “therapy” means a treatment intended to relieve or heal a disorder.

In a further aspect, the present invention also comprises a vector comprising the nucleic acid construct as described elsewhere herein.

As used within the present invention, the term “vector” is a nucleic acid molecule, such as a DNA molecule, which is used as a vehicle to artificially carry genetic material into a cell. The vector is generally a nucleic acid sequence that consists of an insert (such as an heterologous nucleic acid sequence or gene for a transcriptional regulator protein) and a larger sequence that serves as the “backbone” of the vector. The vector may be in any suitable format, including plasmids, mini-circle, or linear DNA. The vector may comprise at least the gene for the transcriptional regulator or heterologous nucleic acid sequence operably linked to an inducible promoter, together with the minimum sequences to enable insertion of the genes into the relevant intron. Optionally, the vectors also possess an origin of replication (ori), which permits amplification of the vector, for example in bacteria. Additionally, or alternatively, the vector includes selectable markers such as antibiotic resistance genes, genes for coloured markers and suicide genes.

In a further aspect, the present invention also comprises a cell comprising the nucleic acid construct or part thereof or the vector as described elsewhere herein.

In the present invention, the term “cell” may be a mature cell type. Such cells are differentiated and specialised and are not able to develop into a different cell type. Mature cell types could be any cell from the human or animal body. It is preferably a mammalian cell, such as a cell from a rodent, such as mice and rats; marsupial such as kangaroos and koalas; non-human primate such as a bonobo, chimpanzee, lemurs, gibbons and apes; camelids such as camels and llamas; livestock animals such as horses, pigs, cattle, buffalo, bison, goats, sheep, deer, reindeer, donkeys, bantengs, yaks, chickens, ducks and turkeys; domestic animals such as cats, dogs, rabbits and guinea pigs. The cell is preferably a human cell. In certain aspects, the cell is preferably one from a livestock animal.

Alternatively, the cells may be a tissue-specific stem cell, which may also be autologous or donated. Suitable cells include epiblast stem cells, induced neural stem cells and other tissue-specific stem cells. In certain embodiments, it may be preferred that the cell used is an embryonic stem cell or stem cell line. Numerous embryonic stem cell lines are now available, for example, WA01 (HI) and WA09 (H9) can be obtained from WiCell, and KhES-1, KhES-2, and KhES-3 can be obtained from the Institute for Frontier Medical Sciences, Kyoto University (Kyoto, Japan). It may be preferred that the embryonic stem cell is derived without destruction of the embryo, particularly where the cells are human, since such techniques are readily available (Young et al., 2008).

The cells used in the method of the present invention may thus be any type of adult stem cells; these are unspecialised cells that can develop into many, but not all, types of cells. Adult stem cells are undifferentiated cells found throughout the body that divide to replenish dying cells and regenerate damaged tissues. Also known as somatic stem cells, they are not pluripotent. Adult stem cells have been identified in many organs and tissues, including brain, bone marrow, peripheral blood, blood vessels, skeletal muscle, skin, teeth, heart, gut, liver, ovarian epithelium, and testis. In order to label a cell as somatic stem cell, the skilled person must demonstrate that a single adult stem cell can generate a line of genetically identical cells that then gives rise to all the appropriate differentiated cell types of the tissue. To confirm experimentally that a putative adult stem cell is indeed a stem cell, the cell must either give rise to these genetically identical cells in culture, or a purified population of these cells must repopulate tissue after transplantation into an animal. Suitable cell types include, but are not limited to, neural, mesenchymal and endodermal stem and precursor cells.

The cells produced according to any of the methods of the invention have applications in diagnostic and therapeutic methods. The cells may be used in vitro to study cellular development, provide test systems for new drugs, enable screening methods to be developed, scrutinise therapeutic regimens, provide diagnostic tests and the like. These uses form part of the present invention. Alternatively, the cells may be transplanted into a human or animal patient for diagnostic or therapeutic purposes. The use of the cells in therapy is also included in the present invention. The cells may be allogeneic (i.e. mature cells removed, modified and returned to the same individual) or from a donor (including a stem cell line).

The present invention also relates to the use of the nucleic acid construct, the vector, or the cell as described elsewhere herein for detecting the cell identity, the cell state or the time point of expression of the nucleic acid construct. In an additional embodiment, the present invention comprises the use of the nucleic acid construct, the vector, or the cell as described elsewhere herein for detecting the expression of a gene of interest, the protein encoded by the gene of interest, the cell identity, the cell state or the time point of expression of the gene of interest.

As used within the present invention, the term “cell identity” means the developmental origin and central features of a mature cell, which distinguish one cell population from another. This may include the gene expression and metabolism of a cell.

In the present invention, the term “cell state” means the current physiological condition and properties of a cell including the expression of genes, epigenetic signatures and metabolism.

In a further aspect, the present invention also comprises the use of the nucleic acid construct, the vector, or the cell as described elsewhere herein for enriching cells.

In a further aspect, the present invention comprises the nucleic acid construct, the vector, or the cell as described elsewhere herein for use in the treatment or prevention of a disease. Preferably, the disease is selected from the group consisting of retinopathies, tauopathies, motor neuron diseases, muscular diseases, neurodevelopmental and neurodegenerative diseases. More preferably, the disease is selected from the group consisting of cystic fibrosis, retinitis pigmentosa, myotonic dystrophy, Alzheimer's disease and Parkinson's disease.

In a further aspect, the present invention also comprises the nucleic acid construct, the vector, or the cell as described elsewhere herein for use in tissue generation, gene therapy and in vitro reprogramming of cells.

As used within the present invention, the term “gene therapy” may be defined as the intentional insertion of foreign DNA into the nucleus of a cell with therapeutic intent. Such a definition includes the provision of a gene or genes to a cell to provide a wild type version of a faulty gene, the addition of genes for RNA molecules that interfere with target gene expression (which may be defective), provision of suicide genes (such as the enzymes herpes simplex virus thymidine kinase (HSV-tk) and cytosine deaminase (CD), which convert the harmless prodrug ganciclovir (GCV) into a cytotoxic drug), DNA vaccines for immunisation or cancer therapy (including cellular adoptive immunotherapy) and any other provision of genes to a cell for therapeutic purposes. Somatic stem cells and mature cell types may be modified according to the present invention and then used for applications such as gene therapy or genetic vaccination.

Typically, the method of the invention may be used for insertion of a desired genetic sequence for transcription in a cell, preferably expression, particularly in DNA vaccines. DNA vaccines typically encode a modified form of an infectious organism's DNA. DNA vaccines are administered to a subject where they then express the selected protein of the infectious organism, initiating an immune response against that protein, which is typically protective. DNA vaccines may also encode a tumour antigen in a cancer immunotherapy approach. A DNA vaccine may comprise a nucleic acid sequence encoding an antigen for the treatment or prevention of a number of conditions, including, but not limited to, cancer, allergies, toxicity and infection by a pathogen, such as, but not limited to, fungi, viruses including Human Papilloma Viruses (HPV), HIV, HSV2/HSV1, Influenza virus (types A, B and C), Polio virus, RSV virus, Rhinoviruses, Rotaviruses, Hepatitis A virus, Measles virus, Parainfluenza virus, Mumps virus, Varicella-Zoster virus, Cytomegalovirus, Epstein-Barr virus, Adenoviruses, Rubella virus, Human T-cell Lymphoma type I virus (HTLV-I), Hepatitis B virus (HBV), Hepatitis C virus (HCV), Hepatitis D virus, Pox virus, Zika virus, Marburg and Ebola; bacteria including Meningococcus, Haemophilus influenza (type b); and parasitic pathogens. DNA vaccines may comprise a nucleic acid sequence encoding an antigen from any suitable pathogen. The antigen may be from a pathogen responsible for a human or veterinary disease and in particular may be from a viral pathogen.

DNA vaccines inserted into the intron may also comprise a nucleic acid sequence encoding tumour antigens. Examples of tumour associated antigens include, but are not limited to, cancer-antigens such as members of the MAGE family (MAGE 1, 2, 3 etc.), NY-ESO-1 and SSX-2, differentiation antigens, such as tyrosinase, gpIOO, PSA, Her-2 and CEA, mutated self-antigens and viral tumour antigens, such as E6 and/or E7 from oncogenic HPV types. Further examples of particular tumour antigens include MART-I, Melan-A, p97, beta-HCG, Gal NAc, MAGE-I, MAGE-2, MAGE-4, MAGE-12, MUCI, MUC2, MUC3, MUC4, MUC18, CEA, DDC, PIA, EpCam, melanoma antigen gp75, Hker 8, high molecular weight melanoma antigen, KI 9, Tyrl, Tyr2, members of the pMel 17 gene family, c-Met, PSM (prostate mucin antigen), PSMA (prostate specific membrane antigen), prostate secretary protein, alpha-fetoprotein, CA 125, CA 19.9, TAG-72, BRCA-I and BRCA-2 antigen. The inserted genetic sequence may produce other types of therapeutic DNA molecules. For example, such DNA molecules can be used to express a functional gene, where a subject has a genetic disorder caused by a dysfunctional version of that gene. Examples of such diseases include Duchenne muscular dystrophy, cystic fibrosis, Gaucher's Disease, and adenosine deaminase (ADA) deficiency. Other diseases where gene therapy may be useful include inflammatory diseases, autoimmune, chronic and infectious diseases, including such disorders as AIDS, cancer, neurological diseases, cardiovascular disease, hypercholestemia, various blood disorders, including various anaemias, thalassemia and haemophilia, and emphysema.

For the treatment of solid tumours, genes encoding toxic peptides (i.e., chemotherapeutic agents such as ricin, diphtheria toxin and cobra venom factor), tumour suppressor genes, such as p53, genes coding for mRNA sequences, which are antisense to transforming oncogenes, antineoplastic peptides, such as tumour necrosis factor (TNF) and other cytokines, or transdominant negative mutants of transforming oncogenes, may be expressed.

The present invention also comprises the nucleic acid construct, the vector, or the cell as described elsewhere herein for use as a medicament.

As used within the present invention, the term “medicament” means a healing substance or remedy used for the treatment of diseases or suboptimal health conditions.

In a further aspect, the present invention also comprises the use of the nucleic acid construct, the vector, or the cell as described elsewhere herein in tissue engineering.

In a further aspect, the present invention also comprises a kit for detecting a nucleic acid construct or part thereof and/or detecting the expression product of the nucleic acid construct or part thereof, wherein the kit comprises:
a. at least one heterologous nucleic acid sequence, which not encodes a protein;
   at least one nucleic acid sequence for transcription of the nucleic acid construct or part thereof, and
   at least one nucleic acid sequence for exporting the nucleic acid construct out of the nucleus,
or
b. at least one heterologous nucleic acid sequence, which encodes a protein,
   at least one nucleic acid sequence for transcription of the nucleic acid construct or part thereof,
   at least one nucleic acid sequence for translation of the nucleic acid construct or part thereof,
   at least one nucleic acid sequence for preventing degradation of the nucleic acid construct or part thereof, and
   at least one nucleic acid sequence for exporting the nucleic acid construct or part thereof out of the nucleus, and
      a second vector coding for a guided endonuclease, preferably wherein the endonuclease is selected from the group consisting of Cas9, Cas12a, TALEN5, ZFNs and meganucleases.

In the present invention, the term “kit” means a set of equipment and substances recapitulating the method of the present invention enabling any person to produce cells containing the nucleic acid construct or the vector disclosed anywhere herein. The same definitions given above with regard to the method of the present invention also apply to the kit of the present invention.

In one embodiment of the kit of the present invention, the at least one nucleic acid sequence for transcription of the nucleic acid construct or part thereof comprises a splice donor nucleic acid sequence and a splice acceptor nucleic acid sequence; preferably wherein the splice donor nucleic acid sequence comprises or consists of SEQ ID NO: 1 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 1) and/or, wherein the splice acceptor nucleic acid sequence comprises or consists of SEQ ID NO: 2 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 2).

Preferably, the splice donor nucleic acid sequence comprises or consists of a sequence being at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% identical or homologue to the SEQ ID NO: 1 as depicted herein.

Preferably, the splice acceptor nucleic acid sequence comprises or consists of a sequence being at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% identical or homologue to the SEQ ID NO: 1 as depicted herein. More preferably, the splice donor nucleic acid sequence comprises or consists of SEQ ID NO: 1 and/or the splice acceptor nucleic acid sequence comprises or consists of SEQ ID NO: 2.

In a further embodiment of the kit of the present invention, the at least one nucleic acid sequence for exporting the nucleic acid construct or part thereof out of the nucleus is a viral sequence, preferably comprises or consists of CTE according to SEQ ID NO: 3 or SEQ ID NO: 25 or 37 or 39 (or a sequence which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 3 or 25 or 37 or 39) and/or comprises or consists of WPRE according to SEQ ID NO: 4 or 42 (or a sequence which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 4 or 42).

Preferably, the respective viral sequence comprises or consists of a sequence being at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% identical or homologue to the SEQ ID NO: 3 as depicted herein.

Preferably, in a further embodiment, the respective viral sequence comprises or consists of a sequence being at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% identical or homologue to the SEQ ID NO: 4 as depicted herein. More preferably, the viral sequence comprises or consists of CTE according to SEQ ID NO: 3 and/or comprises or consists of WPRE according to SEQ ID NO: 4.

In a further embodiment of the kit of the present invention, the at least one nucleic acid sequence for preventing degradation of the nucleic acid construct or part thereof is a poly-A-tail, preferably a synthetic poly-A-tail, more preferably wherein the synthetic poly-A-tail comprises at least 30 adenosines.

In one embodiment of the kit of the present invention, the first plasmid further comprises an internal ribosomal entry site (IRES); wherein the at least one nucleic acid sequence for translation of the nucleic acid construct or part thereof is for translation of the heterologous nucleic acid sequence and is initiated by an internal ribosomal entry site (IRES); preferably the internal ribosomal entry site of the virus Encephalomyocarditis virus (EMCV) according to SEQ ID NO: 5 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 5) or the internal ribosomal entry site of the Hepatitis C virus (HCV) according to SEQ ID NO: 6 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 6); and an open reading frame (ORF).

In one embodiment of the kit of the present invention, the heterologous nucleic acid sequence encodes a protein or enzyme selected from the group consisting of a fluorescent protein, preferably green fluorescent protein; a bioluminescence-generating enzyme, preferably NanoLuc, NanoKAZ, TurboLuc, Cypridina, Firefly, Renilla luciferase, split luciferase, split APEX2 or mutant derivatives thereof; an enzyme, which is capable of generating a coloured pigment, preferably tyrosinase or an enzyme of a multi-enzymatic process, more preferably the violacein or betanidin synthesis process, a genetically encoded receptor for multimodal contrast agents, preferably Avidin, Streptavidin or HaloTag or mutant derivatives thereof; an enzyme, which is capable of converting a non-reporter molecule into a reporter molecule, preferably TEV protease and picornaviral proteases, more preferably rhinoviral 3C proteases and polioviral 3C protease, SUMO proteases and mutant derivatives thereof; an enzyme, which is capable of inactivating a toxic compound, preferably blasticidin-S-deaminase, puromycin-N-acetyltransferase, neomycin phosphotransferase, hygromycin B phosphotransferase and mutant derivatives thereof, an enzyme, which is capable of converting pro-drug/toxin-mediated toxicity, preferably thymidine kinase and mutant derivatives thereof and a small-molecule sensor protein, preferably calmodulin, troponin C, S100 and mutant derivatives thereof.

In another embodiment the present invention relates to an overarching differentiating concept, in which the information encoded in the “synthetic exon” is specifically coupled to the regulation of a specific gene (e.g., specific to the splicing of the synthetic exon), preferably dependent on the regulation of a specific promoter. Exemplary overarching differentiating embodiments of the present invention relate to the method/s of the present invention that are suitable for (e.g., can be used for) physiological monitoring of gene regulation, e.g., for monitoring the coding transcript/s and/or non-coding transcript/s:

In some aspects of the present invention, the methods/compositions/kits of the present invention relate to/comprise an endogenous mRNA; and thus the resulting endogenous protein translated from it is not modified, while other methods modify the mRNA (e.g., IRES) or both, the mRNA and the protein (e.g., P2A). In some aspects, the methods/compositions/kits of the present invention are suitable for monitoring the expression dynamics of non-coding RNA. Accordingly, there is a unique combination of advantages of the methods/compositions/kits of the present invention compared to other known methods. This advantage includes that not only a reporter protein (or RNA) can be expressed in an intron-dependent fashion but also a protein that senses or activates a process in the cell (e.g. in the extreme case cell death). In some aspects, the methods/compositions/kits of the present invention relate to a specific intervention/use that is disclosed in the Cre-dependent invertible polyA signal that leads to a premature termination of transcription but other interventions/uses are also possible.

In some aspects of the present invention, a coding transcript that can be combined with a non-coding RNA code (e.g., barcode), e.g., encoded on the DNA level, that preferably contains information about the intron-specific gene regulation. This barcode may, for example, contain an identifier (ID) of the intron/locus (intron ID), and/or ID of the cell (cell ID), and/or an ID representing a counter or timer (counter ID, timer ID). In some aspects of the present invention, a barcode within the intron may be stabilized via triple helices. In some aspects of the present invention, a barcode within the intron may be stabilized indirectly by stimulating its nuclear export via RNA motifs to escape intron-degradation in the nucleus (e.g., CTE, RTEm26 (mutated version of RTE, CTE from the TAP gene, CAE, WPRE). In some aspects of the present invention, the coding transcript can code for a protein that modifies the polynucleotide of the non-coding RNA code. This may occur at the level of the RNA (e.g., via dead Cas13 (dCas13- and ddCas13-based fusion proteins). dCas13 as used herein may refer to Cas13 protein with mutations that deactivate the HEPN nuclease domains but with an intact pre-crRNA processing domain. ddCas13 (double-dead Cas13) as used herein may refer Cas13 protein with mutations that deactivate the HEPN nuclease domains and also mutation that inactivates the pre-crRNA processing domain. In some aspects of the present invention, the encoded protein of the present invention can also be a DNA-editing enzyme which modifies a polynucleotide on the DNA and/or RNA level using guided nucleases, i.e., by generations of random insertions and deletions (InDel), or a chimeric fusion of a nuclease-dead RNA-guided CRISPR-effector, e.g., Cas9, dCas9 (e.g., nuclease-dead Cas9 mutant that does not exhibit nuclease activity), and nCas9 (e.g., nickase version of Cas9 where one single nuclease domain of the two are inactivated (e.g., inactive RuvC with active HNH domain or active RuvC with inactive HNH domain)), fused to base-editing enzymes, e.g., cytidine deaminases (converts c>t) or adenine base editors (converts a>g), or a chimeric fusion of a DNA-dependent RNA-polymerase fused to the aforementioned base-editing enzymes. Editing efficiency of the chimeric enzymes of the present invention can be enhanced by additional fusions to uracil glycosylase inhibitors (UGI). In some aspects of the present invention, the non-coding RNA code could also encode information that may be acted upon by cellular processes, e.g., via toehold switches or padlock probes, unlocks a specific motif upon an RNA key, e.g., a guide sequence for Cas9, Cas13 and/or Cas12a handle (e.g., sgRNA (Cas9), crRNA (Cas12a, Cas13), pre-crRNA (Cas12a, Cas13) (e.g., Felletti et al., 2016; Nature Communications volume 7, Article number: 12834). In some aspects of the present invention, The RNA/DNA of the present invention may also code for an artificial shRNA or microRNA that is, e.g., repurposed as barcode and is exported during its maturation to the cytosolic compartment.

In some aspects of the present invention, the RNA export motif of the present invention comprises or consists of a sequence being at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% identical or homologous to the SEQ ID NOs: 37 (CTEv4), 39 (CTEv2), 40 (CAE-ml), 41 (RTEm26-m1), 42 (WPRE-m2) or 43 (TAP-CTE-m1) as depicted herein.

In some aspects of the present invention the RNA stabilization motif of the present invention comprises or consists of a sequence being at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% identical or homologous to the SEQ ID NO: 38 (MmuMalat1 triple helix) as depicted herein.

In some aspects of the present invention, hidden splice donor/acceptor site/s are destroyed.

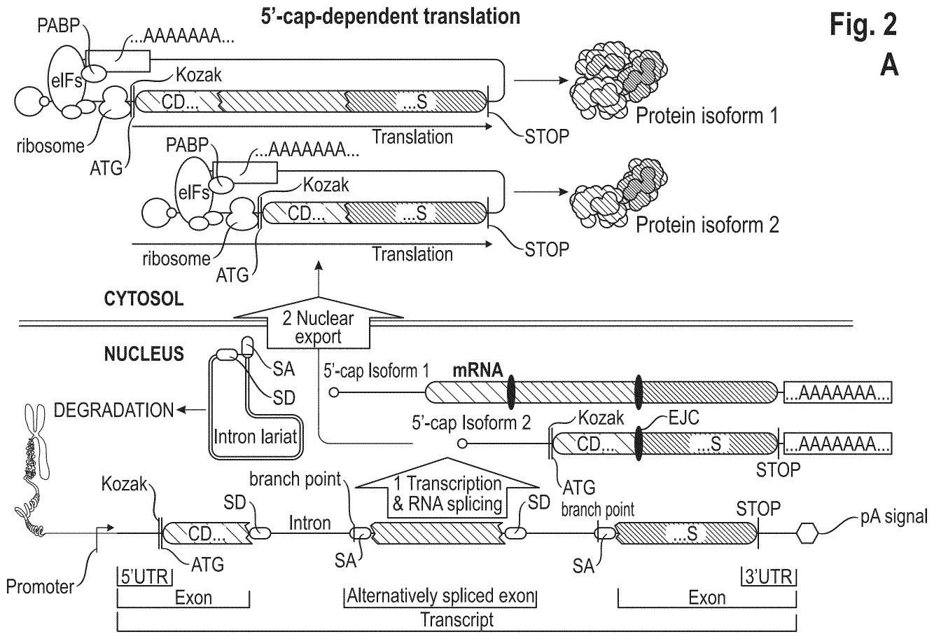

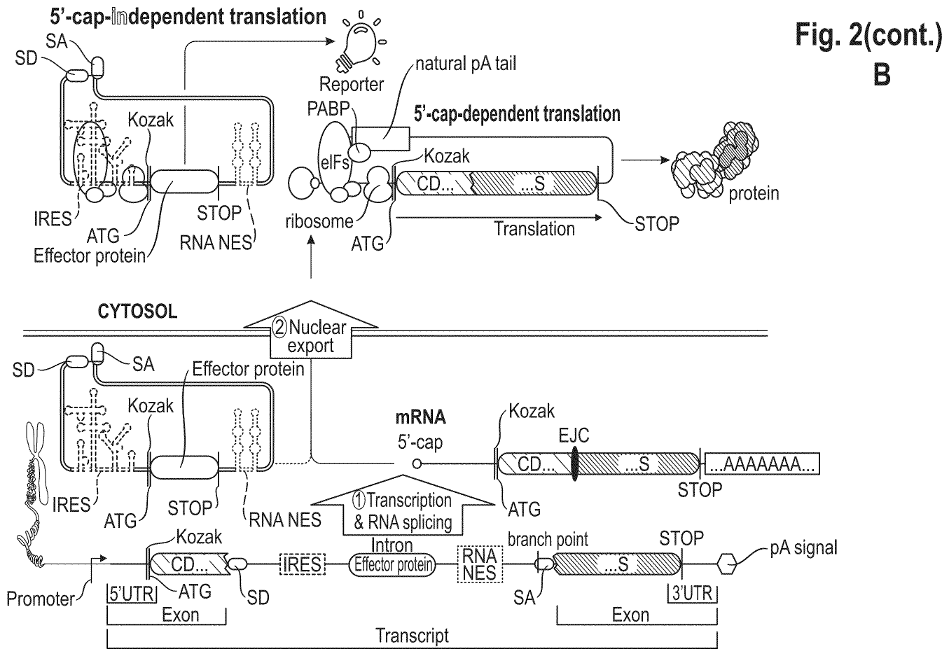

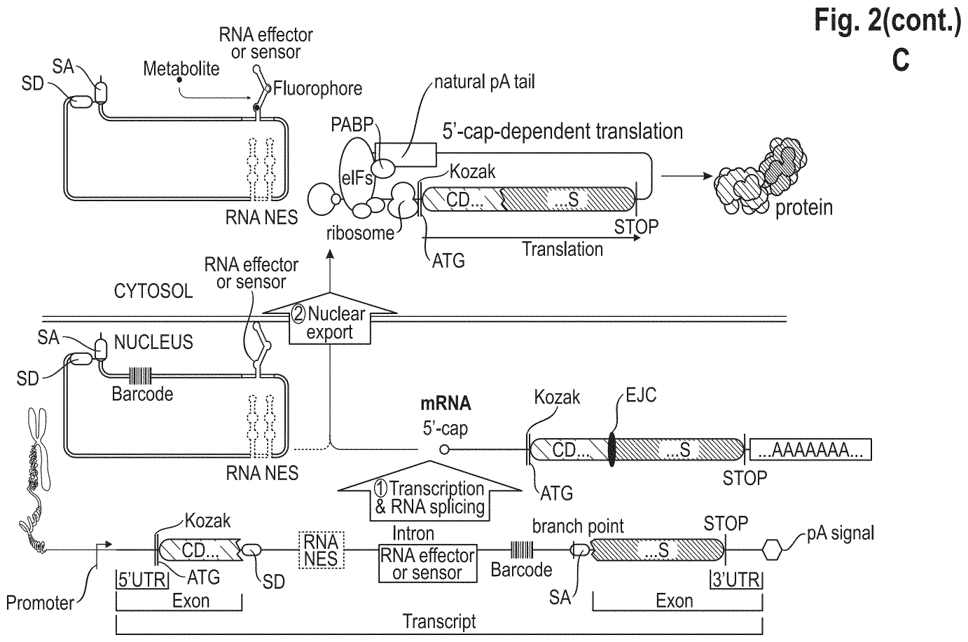

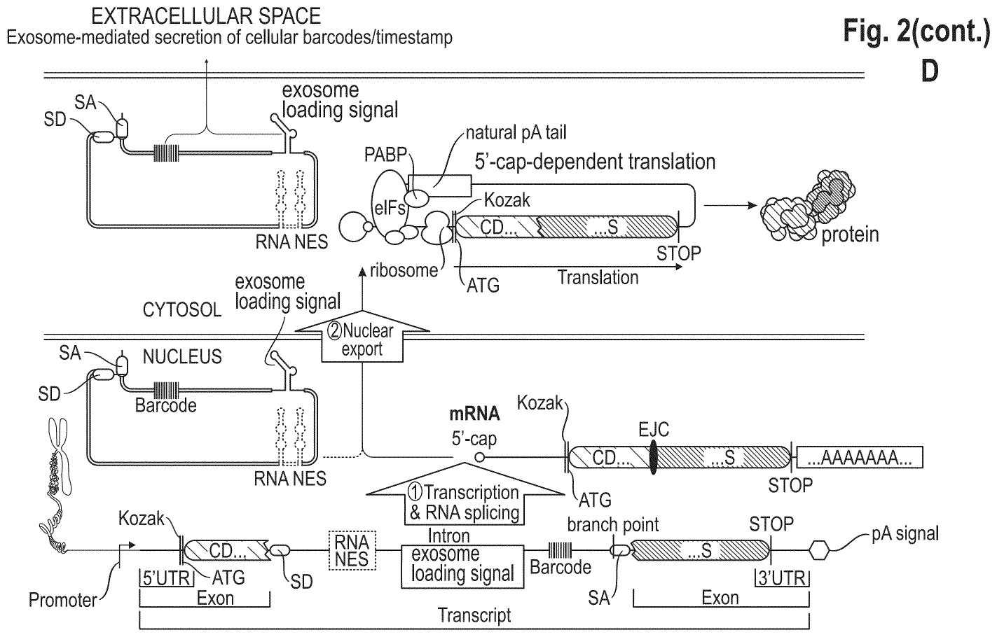

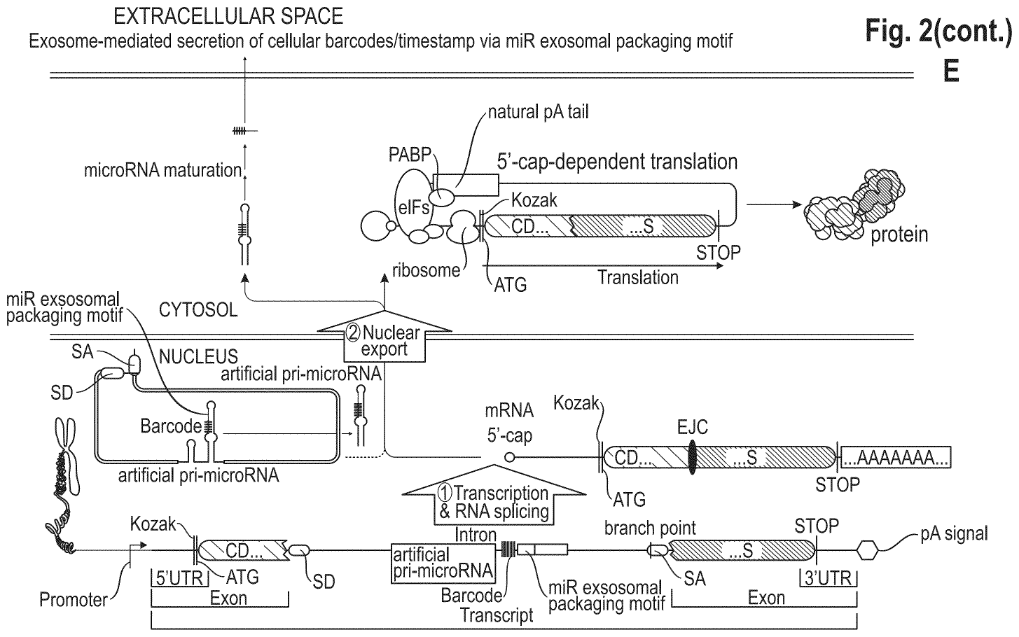

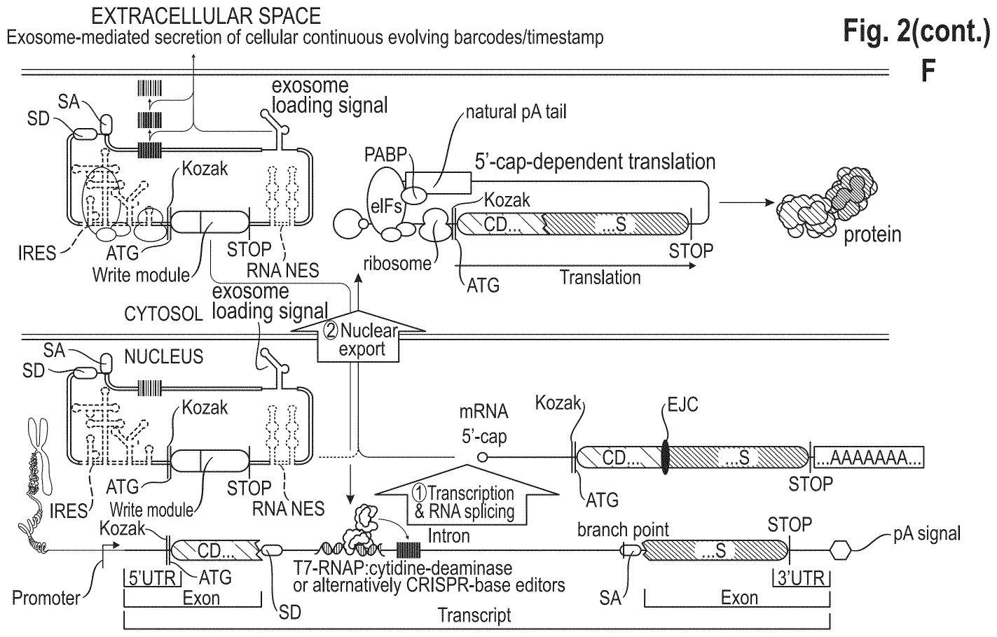

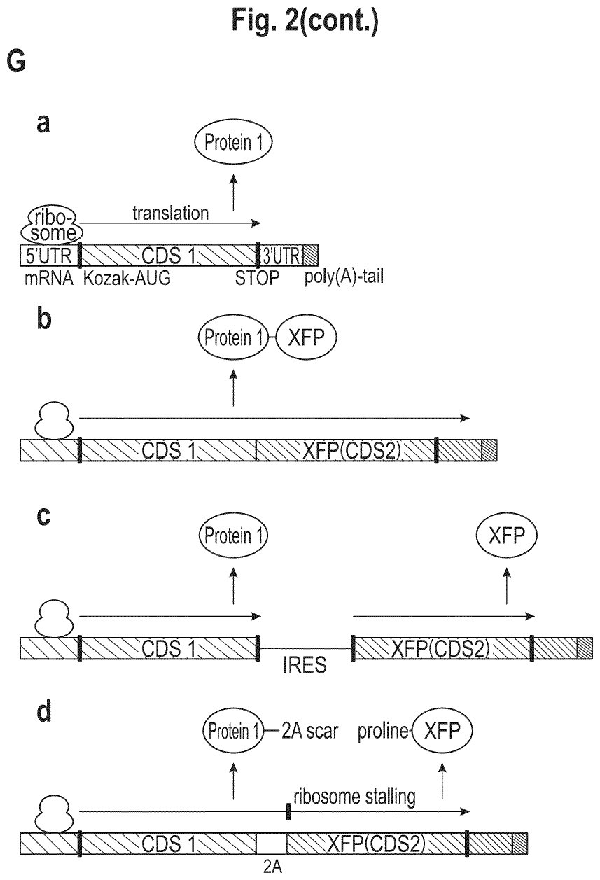

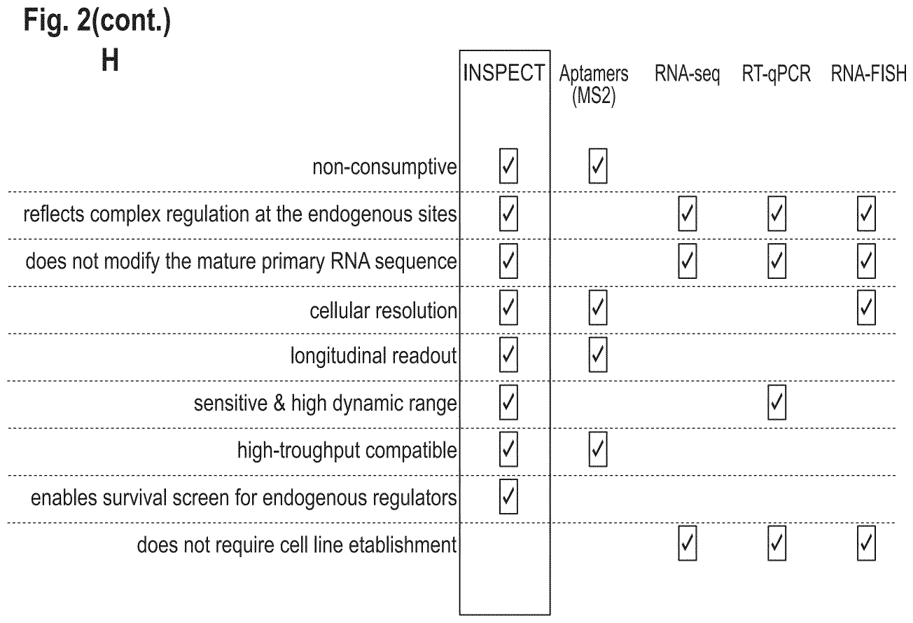

In some aspects of the present invention, the intron-specific transcript can also be secreted from the cell, such that the intron-specific information can be read out via, e.g., RT-qPCR, sequencing and/or in vitro translated into proteins to e.g., obtain multi-time point information. For example, this may be realized by using an “export signal” that is read by an endogenous secretion machinery (e.g., mIR223:Y-box, exosomes)→(e.g., FIG. 2) and/or heterologous or engineered “export signal” that interacts with a heterologous or engineered cell export machinery (examples are MCP:MS2, L7ae:C/Dbox, pumilios, dCas13, (polyA) binding protein, adapters to proteins that cause cell budding (e.g., gag, ARC).

Advantages of the methods/compositions/kits of the present invention include (e.g., FIG. 2h): use for monitoring: gene expression and/or protein translation and/or RNA encoding and/or RNA regulation (e.g., non-invasively/multi-time point, in vitro, ex vivo, in vivo, etc.), wherein said methods/compositions/kits preferably have one or more of the following: non-consumptiveness, capacity to reflect complex regulation at an endogenous site, capacity not to modify a mature primary RNA sequence, cellular resolution, longitudinal readout, sensitive and high dynamic range, high-throughput compatibility, capacity to enable survival screen for endogenous regulator/s. Preferably said monitoring is carried out by the means of PET (positron emission tomography) and/or SPECT (single photon emission computed tomography).

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. The term “at least one” refers, if not particularly defined differently, to one or more such as two, three, four, five, six, seven, eight, nine, ten or more. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number.

For example, less than 20 mean less than the number indicated. Similarly, more than or greater than means more than or greater than the indicated number, e.g. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

The term “about” means plus or minus 10%, preferably plus or minus 5%, more preferably plus or minus 2%, most preferably plus or minus 1%. When used herein, the term “about” may be understood to mean that there can be variation in the respective value or range (such as pH, concentration, percentage, molarity, number of amino acids, time etc.) that can be up to 5%, up to 10% of the given value. For example, if a formulation comprises about 5 mg/ml of a compound, this is understood to mean that a formulation can have between 4.5 and 5.5 mg/ml.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be gained from the following examples, offered for illustrative purposes only. The examples are not intended to limit the scope of the present invention in any way.

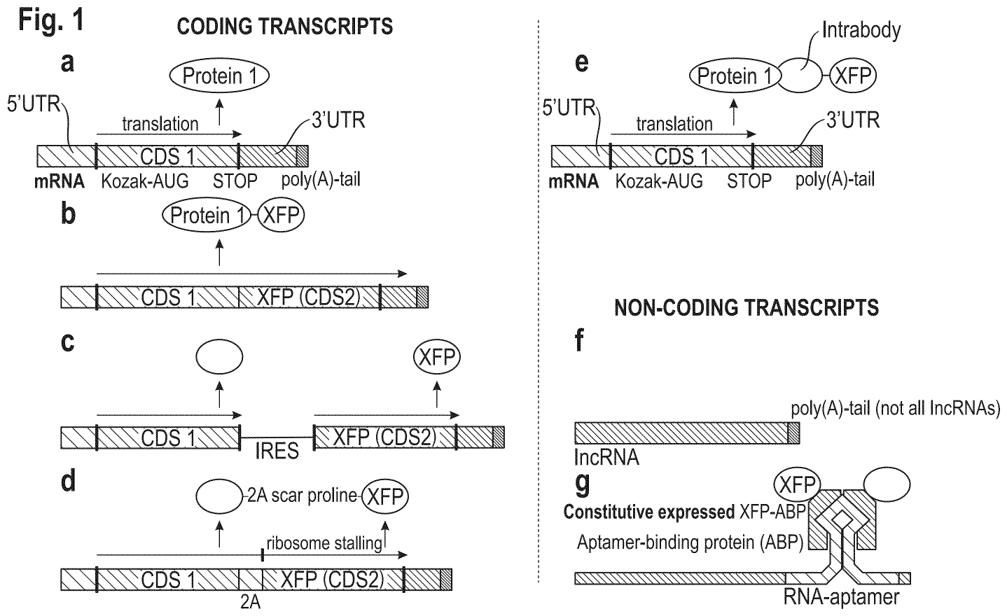

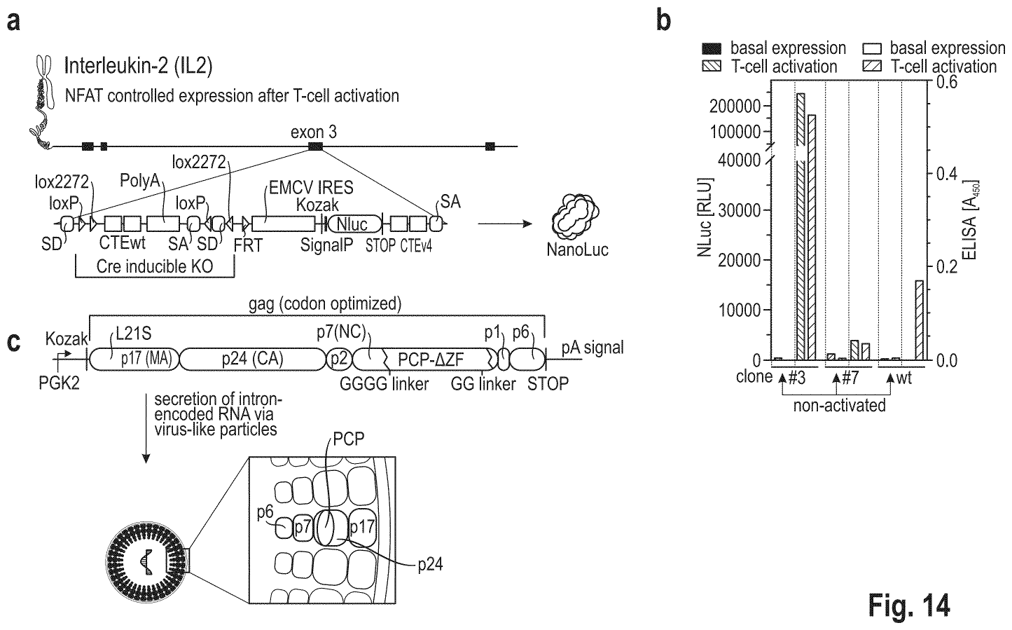

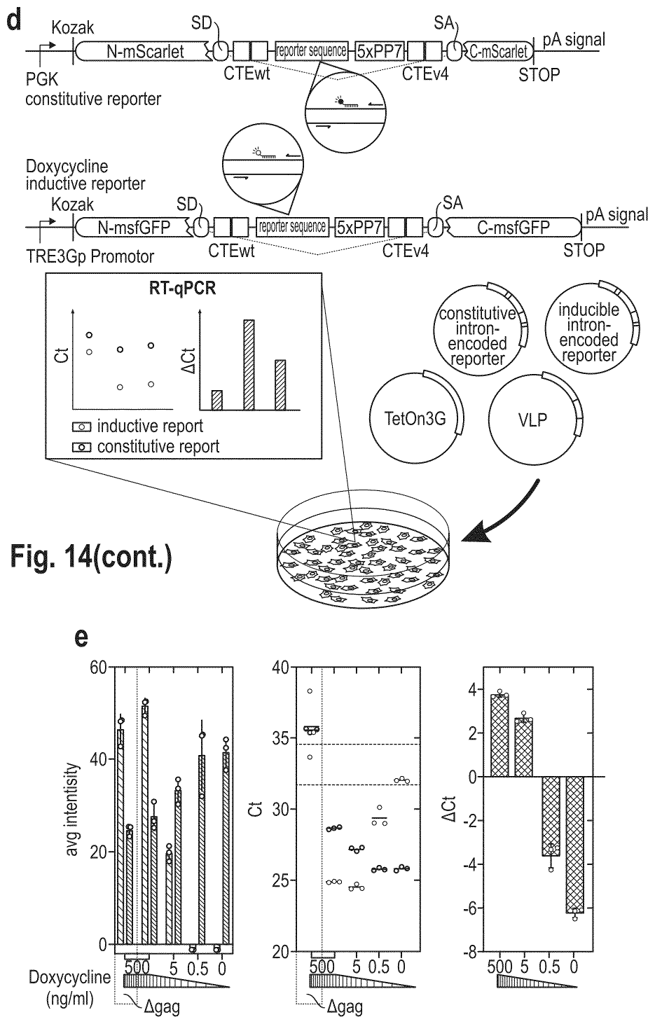

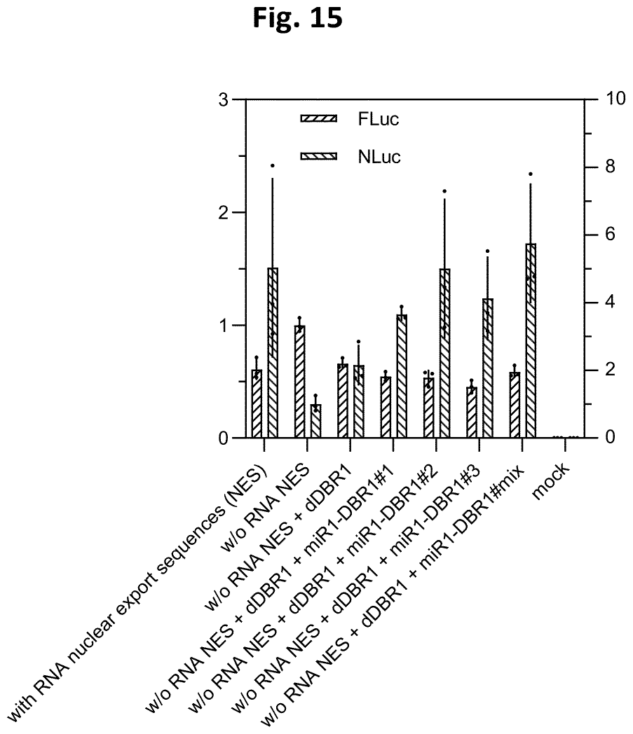

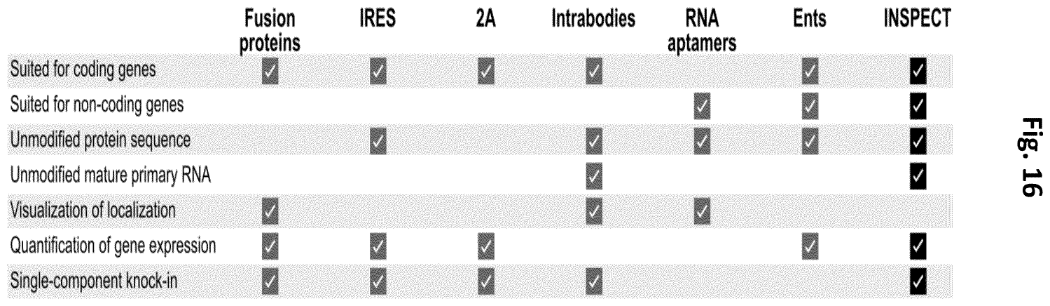

The invention is also characterized by the following items:
1. A method for detecting a nucleic acid (e.g., DNA or RNA) construct or part thereof and/or detecting the expression product of the nucleic acid construct or part thereof, wherein the method comprises inserting a nucleic acid construct or part thereof into an intron or a synthetic intron, wherein the nucleic acid construct comprises:
   a. at least one heterologous nucleic acid sequence, which does not encode a protein;
      at least one nucleic acid sequence for transcription of the nucleic acid construct or part thereof, and at least one nucleic acid sequence for exporting the nucleic acid construct out of the nucleus, or
   b. at least one heterologous nucleic acid sequence, which encodes a protein, at least one nucleic acid sequence for transcription of the nucleic acid construct or part thereof,
      at least one nucleic acid sequence for preventing degradation of the nucleic acid construct or part thereof,
      at least one nucleic acid sequence for exporting the nucleic acid construct out of the nucleus or part thereof, and at least one nucleic acid sequence for translation of the nucleic acid construct or part thereof.
2. Method according to item 1b, wherein the at least one nucleic acid sequence for translation of the nucleic acid construct or part thereof is a nucleic acid sequence for translation of the heterologous nucleic acid sequence.
3. Method according to item 1 or 2, wherein the nucleic acid construct or part thereof is under the control of an endogenous promoter of the gene comprising the expression product of the nucleic acid construct or part thereof.
4. Method according to any one of the previous items, wherein the at least one nucleic acid sequence for transcription of the nucleic acid construct or part thereof comprises a splice donor nucleic acid sequence and a splice acceptor nucleic acid sequence; preferably wherein the splice donor nucleic acid sequence comprises or consists of SEQ ID NO: 1 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 1), and/or, wherein the splice acceptor nucleic acid sequence comprises or consists of SEQ ID NO: 2 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 2).
5. Method according to any one of the previous items, wherein the at least one nucleic acid sequence for exporting the nucleic acid construct or part thereof out of the nucleus is a viral sequence, preferably wherein the at least one nucleic acid sequence for exporting the nucleic acid construct or part thereof out of the nucleus comprises or consists of CTE according to SEQ ID NO: 3 or SEQ ID NO: 25 or 37 or 39 or SEQ ID NO: 44 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 3 or 25 or 37 or 39 or 44) and/or comprises or consists of WPRE according to SEQ ID NO: 4 or 42 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 4 or 42).
6. Method according to any one of the previous items 1 b and 2 to 4, wherein the at least one nucleic acid sequence for translation of the nucleic acid construct or part thereof is for translation of the heterologous nucleic acid sequence and is initiated by an internal ribosomal entry site (IRES); preferably wherein the at least one nucleic acid sequence for translation of the nucleic acid construct or part thereof is the internal ribosomal entry site of the virus Encephalomyocarditis virus (EMCV) according to SEQ ID NO: 5 (or a sequence which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 5) or the internal ribosomal entry site of the Hepatitis C virus (HCV) according to SEQ ID NO: 6 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 6); and an open reading frame (ORF).
7. Method according to any one of the previous items 1 b and 2 to 6, wherein the at least one nucleic acid sequence for preventing degradation of the nucleic acid construct or part thereof is a poly-A-tail, preferably a synthetic poly-A-tail, more preferably, wherein the synthetic poly-A-tail comprises at least 30 adenosines, and even more preferred, wherein the poly-A-tail comprises or consists of the sequence according to SEQ ID NO: 7 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 7).
8. Method according to any one of the previous items 1 b and 2 to 7, wherein the at least one nucleic acid sequence for preventing degradation of the nucleic acid construct or part thereof is a polyadenylation signal, preferably a late SV40 polyadenylation signal or a rabbit beta-globin polyadenylation signal, more preferably the late SV40 polyadenylation signal is mutated to be unidirectional.
9. Method according to item 8, wherein the polyadenylation signals are integrated in the nucleic acid construct in antisense direction and are enclosed with loxP sites and wherein after transcription the inverted polyadenylation signal is not separated from the endogenous gene product.
10. Method according to item 9, wherein after the transcription a Cre recombinase (e.g., SEQ ID NO: 8 or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 8) is administered to the transcript to invert the polyadenylation signals into sense direction.
11. Method according to any one of the previous items, wherein the method is non- or minimally invasive for the expression product of the intron or synthetic intron such that a native and/or fully functional protein is expressed compared to the protein without insertion of the nucleic acid construct or part thereof.
12. Method according to any one of the previous items, comprising the insertion of the nucleic acid construct with targeted transgene insertion.
13. Method according to any one of the previous items, wherein the at least one heterologous nucleic acid sequence encodes for a protein-coding RNA, a non-coding RNA, a miRNA, an aptamer, a siRNA, a synthetic RNA sequence or a barcode for extranuclear detection.
14. Method according to any one of the previous items, wherein the at least one heterologous nucleic acid sequence is detected and enables to detect a specific cell.
15. Method according to any one of the previous items, wherein the at least one heterologous nucleic acid sequence is detected and provides information about the transcriptional regulation of the cell or a time stamp of a cellular process.
16. Method according to any one of the previous items, wherein the heterologous nucleic acid sequence encodes a protein or enzyme selected from the group consisting of a fluorescent protein, preferably green fluorescent protein; a bioluminescence-generating enzyme, preferably NanoLuc, NanoKAZ, TurboLuc, Cypridina, Firefly, Renilla luciferase, split luciferase, split APEX2 or mutant derivatives thereof; an enzyme, which is capable of generating a coloured pigment, preferably tyrosinase or an enzyme of a multi-enzymatic process, more preferably the violacein or betanidin synthesis process, a genetically encoded receptor for multimodal contrast agents, preferably Avidin, Streptavidin or HaloTag or mutant derivatives thereof; an enzyme, which is capable of converting a non-reporter molecule into a reporter molecule, preferably TEV protease and picornaviral proteases, more preferably rhinoviral 3C proteases and polioviral 3C protease, SUMO proteases and mutant derivatives thereof; an enzyme, which is capable of inactivating a toxic compound, preferably blasticidin-S-deaminase, puromycin-N-acetyltransferase, neomycin phosphotransferase, hygromycin B phosphotransferase and mutant derivatives thereof, an enzyme, which is capable of converting pro-drug/toxin-mediated toxicity, preferably thymidine kinase and mutant derivatives thereof and a small-molecule sensor protein, preferably calmodulin, troponin C, S100 and mutant derivatives thereof.
17. Method according to item 15, wherein the method further comprises combining the expression of the protein or enzyme encoded by the heterologous nucleic acid sequence to the natural expression of the gene comprising the nucleic acid construct or part thereof by using the same promotor.
18. Method according to any one of the previous items, wherein the heterologous nucleic acid sequence encodes a resistance gene for cell-toxic compounds, preferably wherein the method additionally comprises detecting the survival of the cells comprising the nucleic acid construct or part thereof, more preferably wherein the resistance gene for cell-toxic compounds is used as a selection marker of the cells comprising the nucleic acid construct or part thereof.
19. Method according to any one of the previous items, wherein the heterologous nucleic acid sequence encodes a Cas enzyme, e.g., selected from the group consisting of: Cas9 (e.g., CRISPR-associated endonuclease Cas9, e.g., having EC:3.1.-.- enzymatic activity and/or SEQ ID NO: 9 or UniProtKB Accession Number/s: Q99ZW2, G3ECR, J7RUA5, A0Q5Y3, J3F2B0, C9X1G5, Q927P4, Q8DTE3, Q6NKI3, A11Q68 or Q9CLT2); Cas12a (e.g., CRISPR-associated endonuclease Cas12a, e.g., having EC:3.1.21.1 and/or EC:4.6.1.22 enzymatic activity and/or UniProtKB Accession Number/s: A0Q7Q2, A0A182DWE3 or U2UMQ6, e.g., U2UMQ6 enzyme and/or its variants/mutants may also referred to as Cas12a/Cpf1 enzymes and/or is/are the preferred Cas12a enzyme/s for use in mammalian systems); Cas12b (e.g., CRISPR-associated endonuclease Cas12b, e.g., having EC:3.1.-.- enzymatic activity and/or UniProtKB Accession Number/s: T0D7A2, e.g., T0D7A2 enzyme and/or its variants/mutants may have temperature optimum at about 48° C. and/or may be the preferred Cas12b enzyme/s for use in non-mammalian systems and/or in organisms able to function at a temperature at about 48° C. and/or about 37° C. (e.g., BhCas12b, e.g., having RefSeq Accession Number: WP_095142515.1 and/or BhCas12b v4 mutant/s comprising: K846R and/or S893R and/or E837G mutations, e.g., using the numbering of WP_095142515.1; e.g., as reported by Strecker et al., 2019; Nat Commun. 2019 Jan. 22; 10(1):212. doi: 10.1038/s41467-018-08224-4)); Cas12c (e.g., CRISPR-associated protein 12c, e.g., selected from the group consisting of: SEQ ID NO: 34 (Cas12c1), SEQ ID NO: 35 (Cas12c2) and SEQ ID NO: 36 (OspCas12c); e.g., as reported by Yan et al., 2019; Science. 2019 Jan. 4; 363(6422):88-91. doi: 10.1126/science.aav7271. Epub 2018 Dec. 6; Cas13a (e.g., CRISPR-associated endoribonuclease Cas13a, e.g., having EC:3.1.-.-enzymatic activity and/or UniProtKB Accession Number/s: C7NBY4, P0DOC6, U2PSH1, A0A0H5SJ89, P0DPB7, E4T0I2 or P0DPB8); Cas13b (e.g., CRISPR-associated protein 13b, e.g., UniProtKB Accession Number/s: E6K398); Cas13d (e.g., CRISPR-associated protein 13d, e.g., UniProtKB Accession Number/s: B0MS50 or A0A1C5SD84); Cas14 (e.g., CRISPR-associated protein Cas14, e.g., GenBank Accession Number/s: QBM02559.1, SUY72868.1, VEJ66719.1, SUY81478.1, SUY85836.1 or STC69301.1); CasX (e.g., UniProtKB Accession Number/s: A0A357BT59); and/or sequences which are at least 60% or more, e.g., at least 65%, at least 70%, at least 75%, at least 80%, at least 85%, at least 90%, at least 95%, at least 96%, at least 97%, at least 98%, at least 99% or 100% identical to the Cas sequences as described herein in item 19 (e.g., having the corresponding Cas enzymatic activity) and fusion proteins thereof.
20. Method according to any one of the previous items, wherein the heterologous nucleic acid sequence encodes an amino acid, which can be metabolized to an antibiotic or derivative thereof, preferably for inducing a genetic system, more preferably for inducing the genetic Tet-On/Tet-OFF system.
21. Method according to any one of the previous items, wherein the heterologous nucleic acid sequence encodes an enzyme of a biosynthesis pathway generating a toxin or a mutant thereof.
22. Method according to any one of the previous items, wherein the heterologous nucleic acid sequence is a suicide gene or a gene, which induces a cell death cascade.
23. Method according to any one of the previous items, wherein the heterologous nucleic acid sequence further comprises a polynucleotide encoding a protein, which functions as an activator of the expression of the gene comprising the nucleic acid construct or part thereof.
24. Method according to any one of the previous items, wherein the heterologous nucleic acid sequence encodes a transcription factor.
25. Method according to item 24, wherein the transcription factor is used to force or refine determination of a stem cell into a defined mature cell.
26. Method according to any one of the previous items, wherein the heterologous nucleic acid sequence encodes a transcriptional regulator or a repressor protein.
27. Method according to any one of the previous items, wherein the heterologous nucleic acid sequence encodes a protein, which is a hormone or has the function of a hormone.
28. Method according to any one of the previous items, wherein the heterologous nucleic acid sequence encodes a protein, which is a receptor, preferably a hormone receptor or a mutant derivate thereof.
29. Method according to any one of the previous items, wherein the heterologous nucleic acid sequence encodes an affinity domain or tag to bind protein, DNA or RNA.
30. Method according to item 29, wherein the protein affinity domain is used to capture the expression product of the nucleic acid construct or part thereof, preferably the expression product of the heterologous nucleic acid sequence.
31. Method according to any one of the previous items, wherein the heterologous nucleic acid sequence encodes an antibody or antibody fragment.
32. Method according to item 31, wherein the antibody or antibody fragment is used to capture the expression product of the nucleic acid construct or part thereof, preferably the expression product of the heterologous nucleic acid sequence.
33. Method according to any one of the previous items, wherein the protein or enzyme encoded by the heterologous nucleic acid sequence is for preventing pathological changes within the cell.
34. Method according to any one of the previous items, wherein: i) said method is suitable for detecting biological function/s, preferably the regulation of tissue and cell generation, more preferably neuro-regeneration; and/or ii) said method is for monitoring gene regulation, e.g., of coding transcripts; and/or iii) in said method a coding transcript is combined with a non-coding RNA code (e.g., barcode), e.g., encoded on the DNA level, that preferably contains information about the intron-specific gene regulation; and/or iv) in said method an intron-specific transcript is secreted from a cell, preferably such that the intron-specific information is readable via, e.g., RT-qPCR, sequencing and/or in vitro translated into proteins, e.g., in order to obtain multi-time point information; and/or v) said nucleic acid construct comprising an RNA export motif (e.g., SEQ ID NOs: 37, 39, 40, 41, 42, 43) and/or RNA stabilization motif (e.g., SEQ ID NO: 38 or SEQ ID NO: 45).
35. Method according to any one of the previous items, wherein said nucleic acid (e.g., DNA or RNA) construct comprises one or more of SEQ ID NO: 1-50 and/or corresponding DNA and/or RNA sequence/s (e.g., both DNA and RNA constructs according to SEQ ID NOs: 1-50 are encompassed by the present invention, e.g., complementary sequences are encompassed by the present invention, e.g., if a DNA sequence is provided a corresponding transcribed RNA sequence is within scope of the present invention, if an RNA sequence is provided, a corresponding reverse-transcribed DNA sequence is encompassed by the present invention) and/or a nucleic acid (e.g., DNA or RNA) encoding polypeptide/s of SEQ ID NOs: 51-54.
36. Nucleic acid (e.g., DNA or RNA) construct comprising or consisting of any of SEQ ID NOs: 1 to 50 and/or a nucleic acid (e.g., DNA or RNA) encoding polypeptide of SEQ ID NOs: 51-54 or sequences which are at least 60% or more, e.g., at least 65%, at least 70%, at least 75%, at least 80%, at least 85%, at least 90%, at least 95%, at least 96%, at least 97%, at least 98%, at least 99% or 100% identical to sequences SEQ ID NOs: 1 to 50 as described herein.
37. Nucleic acid construct according to any one of the preceding items, for use in therapy.
38. Nucleic acid construct according to any one of the preceding items, for use in the treatment or prevention of cancer.
39. A vector comprising any nucleic acid construct according to any one of the preceding items.
40. A cell (e.g., recombinant and/or isolated cell) comprising any nucleic acid construct according to any one of the preceding items or the vector according to any one of the preceding items.
41. Use of any nucleic acid construct according to any one of the preceding items, the vector according to any one of the preceding items or the cell according to any one of the preceding items for detecting the cell identity, the cell state or the time point of expression of the nucleic acid construct.
42. Use of any nucleic acid construct according to any one of the preceding items, the vector according to any one of the preceding items or the cell according to any one of the preceding items for enriching cells.
43. The nucleic acid construct according to any one of the preceding items, the vector according to any one of the preceding items or the cell according to any one of the preceding items for use in the treatment or prevention of a disease, preferably wherein the disease is selected from the group consisting of retinopathies, tauopathies, motor neuron diseases, muscular diseases, neurodevelopmental and neurodegenerative diseases, more preferably selected from the group consisting of cystic fibrosis, retinitis pigmentosa, myotonic dystrophy, Alzheimer's disease and Parkinson's disease.
44. The nucleic acid construct according to any one of the preceding items, the vector according to any one of the preceding items or the cell according to any one of the preceding items for use in tissue generation, gene therapy and in vitro reprogramming of cells.
45. The nucleic acid construct according to any one of the preceding items, the vector according to any one of the preceding items or the cell according to any one of the preceding items for use as a medicament.
46. Use of any nucleic acid construct according to any one of the preceding items, the vector according to any one of the preceding items or the cell according to any one of the preceding items in tissue engineering.
47. Kit for detecting a nucleic acid construct or part thereof and/or detecting the expression product of the nucleic acid construct or part thereof, wherein the kit comprises: a first vector comprising nucleic acid construct or part thereof, which comprises
   a. at least one heterologous nucleic acid sequence, which does not encode a protein;
      at least one nucleic acid sequence for transcription of the nucleic acid construct or part thereof, and at least one nucleic acid sequence for exporting the nucleic acid construct out of the nucleus, or
   b. at least one heterologous nucleic acid sequence, which encodes a protein, at least one nucleic acid sequence for transcription of the nucleic acid construct or part thereof, at least one nucleic acid sequence for translation of the nucleic acid construct or part thereof, at least one nucleic acid sequence for preventing degradation of the nucleic acid construct or part thereof, and at least one nucleic acid sequence for exporting the nucleic acid construct out of the nucleus or part thereof, and a second vector coding for a guided endonuclease, preferably wherein the endonuclease is selected from the group consisting of Cas9 (e.g., SEQ ID NO: 9), Cas12a, TALENs, ZFNs and meganucleases.
48. Kit according to any one of the preceding items, wherein the at least one nucleic acid sequence for transcription of the nucleic acid construct or parts thereof comprise a splice donor nucleic acid sequence and a splice acceptor nucleic acid sequence; preferably wherein the splice donor nucleic acid sequence comprises or consists of SEQ ID NO: 1 (or a sequence which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 1) and/or wherein the splice acceptor nucleic acid sequence comprises or consists of SEQ ID NO: 2 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 2).
49. Kit according to any one of the preceding items, wherein the at least one nucleic acid sequence for exporting the nucleic acid construct or part thereof out of the nucleus is a viral sequence, preferably comprises or consists of CTE according to SEQ ID NO: 3 or SEQ ID NO: 25 or SEQ ID NO: 44 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 3 or 25 or 44) and/or comprises or consists of WPRE according to SEQ ID NO: 4 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 4).
50. Kit according to any one of the preceding items, wherein the first plasmid further comprises an internal ribosomal entry site (IRES); wherein the at least one nucleic acid sequence for translation of the nucleic acid construct or part thereof is for translation of the heterologous nucleic acid sequence and is initiated by an internal ribosomal entry site (IRES); preferably the internal ribosomal entry site of the virus Encephalomyocarditis virus (EMCV) according to SEQ ID NO: 5 (or a sequence, which is at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 5) or the internal ribosomal entry site of the Hepatitis C virus (HCV) according to SEQ ID NO: 6 (or a sequence which are at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 6); and an open reading frame (ORF).
51. Kit according to any one of the preceding items, wherein the at least one nucleic acid sequence for preventing degradation of the nucleic acid construct or part thereof is a poly-A-tail, preferably a synthetic poly-A-tail, more preferably wherein the synthetic poly-A-tail comprises at least 30 adenosines, and even more preferred wherein the poly-A-tail comprises or consist of the sequence according to SEQ ID NO: 7 (or a sequence which are at least 60% or more, e.g., at least 65%, at least 70%, at least 75%, at least 80%, at least 85%, at least 90%, at least 95%, at least 96%, at least 97%, at least 98%, at least 99% or 100% identical to the sequence having SEQ ID NO: 7).
52. Kit according to any one of the preceding items, the heterologous nucleic acid sequence encodes a protein or enzyme selected from the group consisting of a fluorescent protein, preferably green fluorescent protein; a bioluminescence-generating enzyme, preferably NanoLuc, NanoKAZ, TurboLuc, Cypridina, Firefly, Renilla luciferase, split luciferase, split APEX2 or mutant derivatives thereof; an enzyme, which is capable of generating a coloured pigment, preferably tyrosinase or an enzyme of a multi-enzymatic process, more preferably the violacein or betanidin synthesis process, a genetically encoded receptor for multimodal contrast agents, preferably Avidin, Streptavidin or HaloTag or mutant derivatives thereof; an enzyme, which is capable of converting a non-reporter molecule into a reporter molecule, preferably TEV protease and picornaviral proteases, more preferably rhinoviral 3C proteases and polioviral 3C protease, SUMO proteases and mutant derivatives thereof; an enzyme, which is capable of inactivating a toxic compound, preferably blasticidin-S-deaminase, puromycin-N-acetyltransferase, neomycin phosphotransferase, hygromycin B phosphotransferase and mutant derivatives thereof, an enzyme, which is capable of converting pro-drug/toxin-mediated toxicity, preferably thymidine kinase and mutant derivatives thereof and a small-molecule sensor protein, preferably calmodulin, troponin C, S100 and mutant derivatives thereof.
53. Kit according to any one of the preceding items, wherein said kit comprises the nucleic acid construct (e.g., DNA or RNA) according to any one of the preceding items.
54. The method, nucleic acid construct, vector, cell or kit according to any one of the preceding items, for use in monitoring gene expression (e.g., non-invasively, in vitro, ex vivo or in vivo monitoring); preferably said monitoring is carried out at an endogenous site; further preferably said method, nucleic acid construct, vector, cell or kit does not modify a mature primary RNA sequence; most preferably said method, nucleic acid construct, vector, cell or kit is/has one or more of the following: i) non-consumptive; ii) cellular resolution; iii) longitudinal readout; iv) sensitive and/or high dynamic range; v) high-throughput compatibility; vi) capacity to enable survival screen (e.g., cell survival), e.g., for endogenous regulator/s; vii) said monitoring is carried out by the means of PET (positron emission tomography) and/or SPECT (single photon emission computed tomography).
55. The method, nucleic acid construct, vector, cell or kit according to any one of the preceding items, arranged and/or as shown in any FIG. 1-16 herein.
56. The method, nucleic acid construct, vector, cell or kit according to any one of the preceding items, for monitoring of gene expression and/or non-coding RNA, preferably for non-invasive monitoring of gene expression and/or non-coding RNA.
57. The method, nucleic acid construct, vector, cell or kit according to any one of the preceding items, wherein said nucleic construct comprises a synthetic intron, preferably said synthetic intron is recognized as an exon by the cell (e.g., said synthetic intron behaves like an exon within the cell).
58. The method, nucleic acid construct, vector, cell or kit according to any one of the preceding items, wherein said nucleic construct encoding one or more intrabodies (e.g., intrabody is an antibody that works within the cell to bind to an intracellular protein), preferably wherein the respective stoichiometries of said intrabody to a target (e.g., intrabody target) are controlled (e.g., intrabodies are only expressed if the target is expressed).
59. The method, nucleic acid construct, vector, cell or kit according to any one of the preceding items, wherein said method, nucleic acid construct, vector, cell or kit is for exporting a transcript that is non-coding for a gene (e.g., a RNA-barcode that can be secreted by the cellular-export unit based on gag or a guide-RNA for CRISPR effector/s such as Cas13, which act in the nucleus (e.g., with lower priority also Cas9 variants although they have to act in the nucleus).
60. The method, nucleic acid construct, vector, cell or kit according to any one of the preceding items, wherein said non-coding RNA is preferably a guide-RNA, e.g., for CRISPR effector/s, e.g., Cas13.
61. The method, nucleic acid construct, vector, cell or kit according to any one of the preceding items, wherein said method, nucleic acid construct, vector, cell or kit is for exporting an intron-encoded transcript into the cytosol which can then be translated into an effector protein or be used as an RNA-barcode, e.g., for sequence-based analysis of cell states either in the cytosol or after secretion from the cell; or the transcript can also be an effector molecule itself that can influence cellular processes, e.g., as guide RNA for Cas13.

### EXAMPLES OF THE INVENTION

The following Examples illustrate the invention but are not to be construed as limiting the scope of the invention.

Background

For classic mRNA translation in eukaryotes, the “closed-loop” model describes the circularization of the mRNA via the mRNA binding proteins on its 5′-cap and on its 3′-end (FIG. 1). In the reporter system of the present invention, the closed-loop model was mimicked by the IRES on the 5′-end.

Typically, nuclear export of mature mRNA transcripts to the cytoplasm is mediated by binding of several proteins and protein complexes to the mRNA, e.g., the cap-binding complex (CBC, composed CBP20 and CBP80), TAP (NXF1), p15 (NXT1) and the poly(A)-binding protein PABP2 (PAPBN1). Those components stimulate the nuclear export of the mRNA. The splicing machinery removes introns of the pre-mRNA and usually, the 5′-2′ linked intron lariat is debranched by DBR1, followed by exonuclease-mediated degradation.

Nuclear export of an mRNA is followed by translation, where the initiation is described by a scanning model, in which the 40S subunit of the ribosome is recruited initially to the 5′-cap multimeric complex of the mRNA, forming the 43S preinitiation complex (PIC) and migrates until finding the first AUG codon within an optimal consensus (Kozak) sequence.

Since many viral transcripts are neither 5′-capped nor polyadenylated, they exploit an alternative strategy for exporting their transcripts from the nucleus and translation initiation. One prominent example for RNA export is the retroviral REV-RRE system from HIV that mediates its RNA-genome export via a REV-mediated binding and nuclear export in its late life-cycle.

### Experimental Design

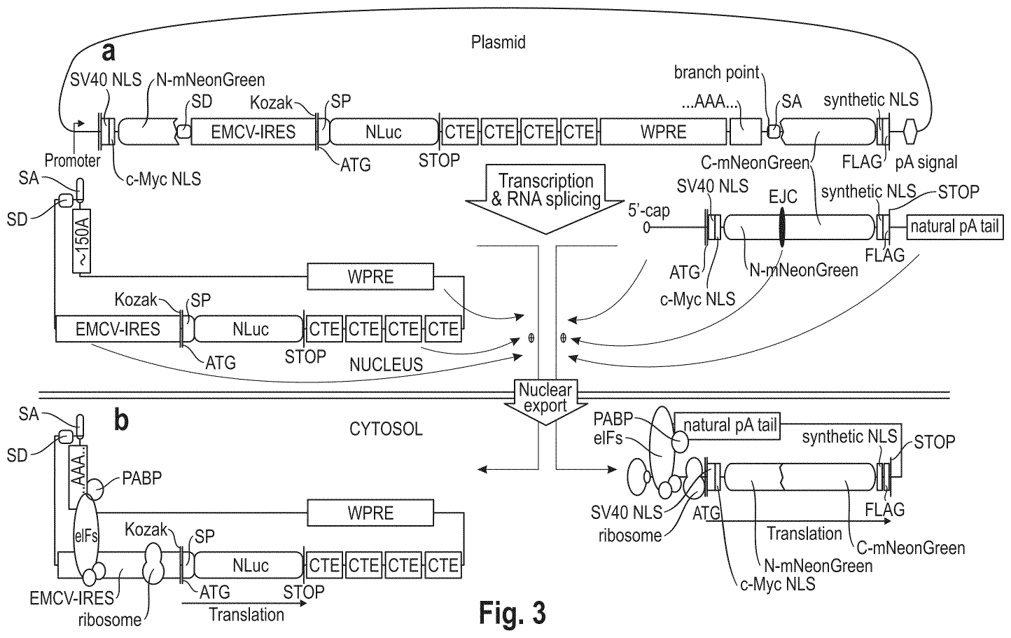

To establish an intron-specific exon-independent coding transcript system, the inventors first created a surrogate reporter comprising a constitutive promoter-driven nuclear-localized fluorescent protein (FIG. 3). The inventors inserted a synthetic intron consisting of a modified rabbit beta-globin intron 1 into the CDS of mNeonGreen (FIG. 3). To test the efficiency of equipping introns with coding sequences, they inserted elements for cap- and poly(A)-independent nuclear export and translation.

To create an export system which only relies on host factors, the inventors used a one-component system from another retrovirus, the Mason-Pfizer monkey virus (MPMV), a region called the constitutive transport element (CTE) on the RNA recruits TAP and p15 from the host export machinery and ensure the export of the viral transcript to the cytoplasm. A better-known system for improving nuclear export of RNA is the Woodchuck Hepatitis Virus (WHP) Posttranscriptional Regulatory Element (WPRE), which has widely been used in transgenic expression systems to enhance mRNA stability and protein yield. WPRE stimulates the nuclear export via karyopherin (CRM1) which explains its positive effect on gene expression on non-polyadenylated transcripts of lentiviral vectors. CRM1 acts as a protein export receptor and exports a subset of endogenous RNAs as well as viral RNAs via adaptor proteins. Translation initiation is mediated in many RNA viruses by an internal ribosome entry site (IRES) located in the 5′-UTR. In contrast to CTE, which is cap-independent but still requires a 5′-3′-direction scanning, an IRES does not require scanning of the ribosome but serves as a ribosome landing pad and promotes cap-independent, internal initiation of RNA translation.

In the present experiments, the inventors compared the IRES efficiencies of hepatitis C virus (HCV) and encephalomyocarditis virus (EMCV). Capped mRNAs recruit the eIF4F complex (consisting of eIF4E, eIF4A, and eIF4G) to the 5′-cap, which allows binding of the 43S pre-initiation complex (40S ribosomal subunit-eIF3-Met-tRNAᵢ-eIF2-GTP-eIF1-eIF1A) and initiation of the scanning process (FIG. 2 a-f). FIG. 2a shows canonical gene expression of most protein-coding genes are driven by an RNA-polymerase II promoter, and 95% of them contain introns that are excised co-/post-transcriptionally leaving the remaining exons ligated scarlessly. This mechanism is called RNA-splicing and is one of the major steps beside 5′-capping (addition of a 7-methylguanylate cap to the 5′-end of the de-novo transcribed RNA) and 3′-polyadenylation (addition of poly(A) tail to the RNA) resulting in a mature mRNA. Some exons are alternatively spliced resulting in isoforms with and without this exon. A complex called exon-junction-complex (EJC) will mark the position ˜50 nt upstream of an exon-exon-junction after splicing. Afterward, a variety of proteins binds to the 5′-cap and the poly(A) tail stimulating the nuclear export of the mature mRNA. The excised intron is degraded after the 2′-5′ phosphodiester bonds of the circular intron is debranched by DBR1. Afterward, the exported mRNA, the 5′-cap-binding, and poly(A) binding proteins initiate translation of the CDS by recruiting the ribosomal subunits. The 5′- and 3′-untranslated region (upstream of the start codon ATG and downstream of the stop codon TAA/TGA/TAG) are called 5′-UTR and 3′-UTR. FIG. 2b shows a scheme of gene transcription and transcript modification and export equipped with an intron-encoded protein translation system. The internal ribosome entry site enables 5′-cap-independent translation of an effector protein that can encode proteinogenic reporters and/or sensors. The RNA nuclear export signal/motif enables 5′-cap-, polyA-, and EJC-independent export of the intronic RNA that is degraded otherwise. FIG. 2c shows a scheme of gene transcription and transcript modification and export equipped with an intron-encoded RNA-effector, more specifically an RNA-sensor or -reporter system. Shown here is an exemplary sensor-effector that encodes an aptamer that fluoresces (reporter) upon a specific metabolite (sensor) using an otherwise non-fluorogenic fluorophore. The RNA nuclear export signal/motif enables the export of the intronic RNA that is degraded otherwise inside the nucleus. FIG. 2d shows a scheme of gene transcription and transcript modification and export equipped with an intron-encoded RNA-barcode, that is additionally exported via the exosomal secretion pathway using motifs (exosomal loading motifs) facilitating exosomal packaging. The RNA nuclear export signal/motif enables the export of the intronic RNA that is degraded otherwise inside the nucleus and thereby enables the packaging of the barcode into exosomes using the exosomal ZIP-code. Readout of the Barcodes is performed using RT followed by NGS or other single-cell sequencing formats that is also compatible to sequence single exosomal vesicles. FIG. 2e is a modification of FIG. 2d where the barcode is embedded within an artificial microRNA that contains a microRNA-specific exosomal targeting motif that enables the secretion of microRNAs via the exosomal pathway. FIG. 2f is a combination of FIGS. 2b and 2d. It combines the proteinogenic coding capability with the RNA-barcoding system. The encoded protein is a DNA-modifying enzyme that preferentially modifies the DNA via base-editing and thereby is evolving the barcode. Depending on the base-editing frequency, the barcodes act as a unique cellular identifier (slow mutation rate) or as a timestamp (fast mutation rate). Similar to 2d, the secreted continuously evolving barcodes are readout via RT followed by NGS or other sequencing technologies such as single-cell transcriptome sequencing technologies. FIG. 2g shows the types of intron-specific information that can be encoded either at the RNA or protein level to serve as a reporter, sensor, or actuator. FIG. 2h tabulates the advantages of the disclosed method for non-invasive monitoring of gene expression.

The EMCV-IRES recruits the 43S particle through direct interaction between the IRES, whereas the HCV-IRES specifically recognizes the 40S subunit and eIF3 (FIG. 3).

The described process enhances mRNA stability and the probability of translation re-initiation. The model proposes that the initiation factors PABP and the eukaryotic translation initiation factor 4E (eIF4E) bind to the 3′-poly(A)-tail and the 5′-cap, respectively, while eIF4G acts as an adaptor protein in-between.

In the reporter system of the present invention, the closed-loop model was mimicked by the IRES on the 5′-end, which recruits the 40S subunit of the ribosome indirectly via a cap-independent binding of translation initiation factors (e.g., EMCV IRES), or directly (e.g., HCV IRES), on the other site (3′-end) by encoding a polyadenylic acid polymer (poly(A)) on the 3′-end of the intron, which recruits PABP and circularizes to the 5′-end. The poly(A) tail was directly encoded and not inserted as a poly(A)-signal which would lead to transcription termination and thus the KO of the host-gene. This aspect was crucial because the intronic reporter should not have an impact on the transcription of the tagged gene of interest. Also, the circular and covalently linked intron lariat mimics the closed-loop state of a translation-competent mRNA and should therefore be beneficial for translation.

Optimization of the System

The inventors carefully designed a minimal set of constructs with mNeonGreen (mNG) as a constitutively expressed exon-encoded protein. They inserted a synthetic intron derived from the first intron of rabbit β-globin gene and inserted it into mNG between Gln-849 and Val-850 (CAGIGTG) since those nucleotides follow the consensus sequence flanking an intronic sequence and thus ensure optimal splicing efficiency. As an intron-encoded protein, they used NanoLuc luciferase (NLuc) with an N-terminal secretion peptide (SP) from Gaussia princeps luciferase. The inventors permuted and combined different elements enabling cap-independent translation and cap- and poly(A) independent nuclear export elements and tested it transiently in HEK293T cells (FIG. 4a). First, they examined the IRES from the hepatitis C virus combined with different nuclear export elements. When transiently transfected in cells, the inventors noticed a time-dependent increase of NLuc signal in the supernatant with different slopes. As expected for SP-NLuc with HCV-IRES, only a marginal increase could be detected. Most likely, the intron escaped the nuclear compartment during cell division and was then translated cap-independently via the HCV-IRES (FIG. 4b). By contrast, inserting also WPRE downstream of HCV-IRES_SP-NLuc, the “intron-encoding capacity (IEC)” could be dramatically increased. CTE elements also have an equal or even better effect since a tandem CTE pair alone downstream of the HCV-IRES_SP-NLuc raised the signal even more than WPRE alone (FIG. 4b). Addition of directly encoded poly(A)s downstream of the elements also increased the signal. The highest signal was measured with all components combined (FIG. 4b). Next, the inventors tested if the IRES from the encephalomyocarditis virus would be more potent to drive cap-independent translation since it has been recently shown that EMCV IRES not only recruits the ribosomal subunits indirectly by the translation initiation factors but also recruits the 40S ribosomal subunit in the absence of eIF4G/4A. Additionally, the commonly used EMCV-IRES (e.g., pCITE-1, pIRES) contained non-optimal mutant variants of the IRES, such as an adenine insertion in the bifurcation loop, and thus are attenuated. The inventors used repaired mutant-free EMCV-IRES and replaced the HCV-IRES for some key constructs. They saw that the EMCV-IRES drove the SP-NLuc translation much more efficiently compared to HCV-IRES since the EMCV-counterpart with only a single WPRE-element already was comparable as the best candidate with HCV-IRES (FIG. 4c). Additionally, equipped with CTE elements and poly(A)s, the signal almost tripled (FIG. 4c). All constructs tested showed a similar expression of the exonic mNeonGreen, indicating the non-invasiveness of those reprogrammed introns (FIG. 4d). FIG. 4e shows the optimization of the nuclear export motifs and stabilizing motifs using a dual-luciferase system. The intron-encoded NanoLuc within the intron is inserted into the firefly luciferase CDS. After transfection, the intron is spliced out and exonic FLuc, as well as intronic NLuc, are expressed separately. Two days post-transfection dual-luciferase assay is performed for evaluation of the results. PEST degradation signal is fused to both, NanoLuc and firefly luciferase, to destabilize the luciferases for a more dynamic signal response. Malat1 triple helix was also tested which stabilizes the 3′-end of a linear RNA. CTEv4, SEQ ID NO: 37 is a variant of CTE without a potential detrimental cryptic splice donor. MmuMalat1 triple helix (SEQ ID NO: 38) is an RNA-stabilizing motif that is derived from the lncRNA Malat1 that protects the 3′-end from degradation. FIG. 4f shows the results from the optimization of the nuclear export motifs and stabilizing motifs from FIG. 4e. FLuc (exonic signal) indicates the integrity of the exon and thus the RNA-splicing itself. NLuc (intronic signal) indicates the nuclear export and translation efficiency of the otherwise degraded intron. Construct IDs 3 and 4 were 20-30-fold better compared to the control construct without nuclear export or stabilization motifs.

Modularity of the Intron-Encoded Protein

After optimization of the intron-encoding capability of the system, the inventors wondered if more complex proteins could be intronically expressed. They selected the sodium-iodide symporter (NIS alias SLC5A5), a multipass transmembrane protein which was inserted into the membrane at the endoplasmic reticulum, as a complex IEP. The expression of NIS could be monitored by measuring the accumulation of radioactive iodine (131I−), which was normally not absorbed by non-thyroid cells (FIG. 5a). SP-NLuc was used as an intron-encoded protein for control. After 48 h post-transfection, the cells were incubated at the specified times, and the accumulated iodine was read out via a γ-scintillator (FIG. 5a). Cells transfected with the intron-encoded NIS showed a dramatic incubation-time-dependent increase in accumulated radioactivity (FIG. 5b), which shows that complex multipass transmembrane proteins can also be encoded in the intron.

Surprisingly, the 3-fold larger size of NIS compared to SP-NLuc did not change the splicing efficiency, as shown by the comparable fluorescence of the exon-encoded nuclear mNG (FIG. 5c) indicating the general usability of introns to encode proteins. The intron-encoded NIS may already prove to be a valuable tool for tracking genes with non-invasive imaging. Besides the 131I−, there are also isotopes such as 124I− (β− and β+ emitter), which are excellent isotopes for positron emission tomography imaging. For example, engineered (CAR)-T-cells could be tracked non-invasively in pre-clinical or clinical settings, where the reporter could be inserted into IL2, an early response marker for activated T-cells. Those activated (CAR)-T-cells express the NIS without the gene for IL2 being modified at the mRNA level since the reporter system is excised at the pre-mRNA level and was translated independently (FIG. 5d). Also, NIS is not immunogenic because it was a human protein unchanged in its sequence, which eases its usage under clinical settings.

Design and Integration of a Non-Leaky and Efficient KO-Switch

Many biological questions regarding the physiological function of a gene are still solved by classic (conditional) knock-outs (KOs). Thus, the inventors sought not only to have an intron-encoded protein but also integrate a knock-out-switch into the system in a way that does not disturb the host gene in its non-activated basal state. For this purpose, the off-switch was placed upstream of the IRES, consisting of the following elements: three inverted poly(A) signals composed of those of the SV40 late poly(A) signal, the rabbit β-globin poly(A) signal and a synthetic poly(A) signal (FIG. 6a). As the SV40 late poly(A) signal also encodes a poly(A) signal in the reverse complementary direction (early poly(A) signal), two mutations were introduced which destroyed the two AAUAAA motifs in the early poly(A) direction. Additionally, an inverted splice acceptor from the second rabbit β-globin intron was placed downstream of the inverted triple poly(A) signal (FIG. 6a). Two semi-orthogonal loxP sites (loxP-WT and lox2272, both are recognized by the Cre recombinase but can only recombine with its sequence-identical site but not with each other; so a semi-orthogonal system) are positioned upstream and downstream of the inverted SA_3×poly(A) in a way, that upon Cre recombinase expression, the inverted SA_3×poly(A) was re-inverted into its active functional state, resulting in a KO of the host-gene. The splice acceptor (SA) ensures that upon Cre-mediated activation of the KO-switch, the usage the poly(A) signals for transcript termination. Without the proximal SA, the poly(A) site could potentially be skipped without being cleaved, since splicing of the intron splice donor (SD) and acceptor of the system are highly efficient and might be faster than the poly(A)-signal-mediated cleavage resulting in a functional host mRNA/ncRNA. The SA of the SA_3×poly(A) ensures the usage of the poly(A) by preventing the usage of the downstream SA of the original intron-encoded construct. The off-switch was placed upstream of the IRES to not only couple the on/off-state to the host gene but also the intron encoded protein to this switch. To facilitate easy selection of cells containing the system in the gene of interest, the inventors couple an inverted EF1α-promoter-driven puromycin N-acetyltransferase (PuroR) and Herpex simplex thymidine kinase (HSV-Tk) expression cassette downstream of the inverted poly(A) signal enabling puromycin-mediated selection. Afterward, the cassette was removed upon FIp recombinase expression, and the cells were counter-selected with ganciclovir. Ganciclovir killed cells that still contained the cassette, because HSV-TK converts ganciclovir to a DNA-damaging agent.

The inventors tested this KO-switch again transiently in the exonic mNeeonGreen-NLS system and co-expressed Cre or FIp recombinases to benchmark the KO-efficiency (FIG. 6a). Upon FIp recombinase expression, both the mNeonGreen and the NLuc activity in the supernatant increased, which can be explained by the excision of the inverted EF1α-driven cassette, the transcriptional interference of the CAG-driven mNeonGreen by the EF1α-promoter does not occur anymore (FIG. 6b,d,e). Upon Cre recombinase expression, the exonic mNeonGreen signal and the intronic NLuc signal was dramatically decreased, indicating an efficient Cre-mediated off-switch (FIG. 6c,d,e).

### Example 1: Non-Invasive Transcriptional Coupling of the lncRNA NEAT1 Using the Reporter System

Ultimately, the inventors wanted to showed that they can transcriptionally couple a non-coding RNA non-invasively via the system to a secretory luciferase and knock it out afterward via Cre recombinase. They selected the long non-coding RNA (lncRNA) NEAT1, which plays a role in pluripotency maintenance in human iPSC and ESC by controlling the phase separation of TDP-43 (TARDBP). NEAT1 is expressed in two isoforms, the short (v1, 3.7 kbp, RefSeq Accession Number: NR_028272.1) and the long version (v2, 22.7 kbp, RefSeq Accession Number: NR_131012.1). TDP-43, which usually shows an increased expression in stem cells, stimulating the premature polyadenylation of NEAT1_v1, thus exclusively expressing v1. If the level of TDP-43 decreases during cell differentiation, NEAT1_v2 is also expressed more frequently because the alternative poly(A) site (APA) of NEAT1_v1 is used less. Since NEAT1_v2 is an essential part of so-called nuclear bodies called paraspeckles (an agglomeration of NEAT1 RNA and sequestered proteins), differentiation also will induce paraspeckle formation. Since NEAT1_v2 also contains elements which bind TDP-43, induction of NEAT1_v2 leads to the phase separation of TDP-43, thus the expression of NEAT1_v2 triggers a positive feedback loop where more and more TDP-43 is taken from the solution and is sequestered into paraspeckles. NEAT1 is also induced in a variety of cellular stress, such as viral infections, DNA damage, in cancer, hypoxia, and heat shock.

The inventors introduced the reporter SP-NLuc using CRISPR/Cas9 into the shared region of NEAT1_v1 and NEAT1_v2 (FIG. 7a). After successful knock-in and selection (puromycin), and FIp-mediated cassette excision (FIG. 7b) and counter-selection (Ganciclovir), only homozygous clones were used for further analysis. A subclone with homozygous NEAT-KO was also created by transfecting a homozygous clone with a plasmid expressing Cre recombinase (FIG. 7c). Using smFISH analysis, the inventors showed that both the reporter clone and unmodified HEK293T cells have paraspeckles, but not the subclone with Cre, where the inverted SA_3×poly(A) signal was flipped in its sense direction. Consequently, the NLuc signal was also barely detectable in the KO clone, clearly demonstrating a transcriptional coupling between the gene and that of the intron-encoded reporter. At the same time, it was also shown that the protein encoded in the intron has no relevant upstream promoter-like sequences that generate false-positive background luciferase activity. Otherwise, a residual signal would be evident despite Cre recombinase. The quantification of the images of the reporter clone and unmodified HEK293T cells (representative examples shown in FIG. 7d) also showed that the number of paraspeckles-containing cells remained unchanged (FIG. 7f).

2. Material and Methods

2.1 Molecular Cloning

PCR for Molecular Cloning

Single-stranded primer deoxyribonucleotides were diluted to 100 μM in nuclease-free water (Integrated DNA Technology (IDT)). PCR reaction with plasmid and genomic DNA template was performed with Q5 Hot Start High-Fidelity 2× Master Mix or with 5× High-Fidelity DNA Polymerase and 5× GC-enhancer (New England Biolabs (NEB)) according to manufacturer's protocol. Samples were purified by gel DNA agarose gel electrophoresis and subsequent purification using Monarch® DNA Gel Extraction Kit (NEB).

DNA digestion with restriction endonucleases: Samples were digested with NEB restriction enzymes according to the manufacturer's protocol in a total volume of 40 μl with 2-3 μg of plasmid DNA. Afterward, fragments were gel-purified by gel DNA agarose gel electrophoresis and subsequent purification using Monarch® DNA Gel Extraction Kit (NEB).

Molecular cloning using DNA ligases and Gibson assembly: Agarose-gel purified DNA fragment concentrations were determined by a spectrophotometer (NanoDrop 1000, Thermo Fisher Scientific). Ligations were carried out with 50-100 ng backbone-DNA (DNA fragment containing the ori) in 20 μl volume, with molar 1:1-3 backbone:insert ratios, using T4 DNA ligase (Quick Ligation™ Kit, NEB) at room temperature for 5-10 min. Gibson assemblies were performed with 75 ng backbone DNA in a 15 μl reaction volume and a molar 1:1-5 backbone:insert ratios, using NEBuilder® HiFi DNA Assembly Master Mix (2×) (NEB) for 20-60 min at 50° C.

DNA agarose gel electrophoresis: Gels were prepared with 1% agarose (Agarose Standard, Carl Roth) in 1×TAE-buffer and 1:10.000 SYBR Safe stain (Thermo Fisher Scientific), running for 20-40 min at 120 V. For analysis 1 kb Plus DNA Ladder (NEB) was used. Samples were mixed with Gel Loading Dye (Purple, 6×) (NEB).

2.2 Bacterial Strains (E. coli) for Molecular Cloning

Chemically- and electrocompetent Turbo/Stable cells (NEB) were used for transformation of circular plasmid DNA. For plasmid amplification, carbenicillin (Carl Roth) was used as a selection agent at a final concentration of 100 μg/ml. All bacterial cells were incubated in Lysogeny Broth-Medium (LB) and on LB agar plates including the respective antibiotics.

2.3 Bacterial Transformation with Plasmid DNA

For electroporation, either 1-5 μl Ligation or Gibson reaction was dialyzed against MilliQ water for 10-20 min on an MF-Millipore membrane filter (Merck). Afterward, 1-5 μl dialysate was mixed with 50 μl of thawed, electrocompetent cells, transferred to a pre-cooled 2 mm electroporation cuvette (Bio-Rad), shocked at 2.5 kV (Gene Pulser Xcell™ Electroporation Systems, Bio-Rad), and immediately mixed with 950 μl SOC-medium (NEB). The chemical transformation was performed by mixing 1-5 μl of Ligation or Gibson reaction with 50 μl thawed, chemically competent cells and incubated on ice for 30 min. Cells were then heat shocked at 42° C. for 30 s, further incubated on ice for 5 min, and finally mixed with 950 μl SOC-medium (NEB). Transformed cells were then plated on agar plates containing an appropriate type of antibiotic and concentrations according to the supplier's information. Plates were incubated overnight at 37° C. or over 48 hours at room temperature.

2.4 Plasmid DNA Purification and Sanger-Sequencing

Plasmid DNA transformed clones were picked and inoculated from agar plates in 2 ml LB medium with appropriate antibiotics and incubated for about 6 h (NEB Turbo) or overnight (NEB Stable). Plasmid DNA intended for sequencing or molecular cloning was purified with QIAprep Plasmid MiniSpin (QIAGEN) according to the manufacturer's protocol. Clones that were intended to be used in cell culture experiments were inoculated in 100 ml antibiotic-medium and grown overnight at 37° C. containing the appropriate antibiotic. Plasmid DNA was purified with the Plasmid Maxi Kit (QIAGEN). Plasmids were sent for Sanger sequencing (GATC-Biotech) and analyzed by Geneious Prime (Biomatters) sequence alignments.

2.5 Mammalian Cell Culture

Cell Lines and Cultivation

HEK293T cells (ECACC: 12022001, Sigma-Aldrich) were maintained at 37° C., in 5% CO₂, H₂O saturated atmosphere were in advanced Gibco™ Advanced DMEM (Gibco™, Thermo Fisher Scientific) supplemented with 10% FBS (Gibco™, Thermo Fisher Scientific), GlutaMAX (Gibco™, Thermo Fisher Scientific) and penicillin-streptomycin (Gibco™, Thermo Fisher Scientific) at 100 μg/ml at 37° C. and 5% CO2. Cells were passaged at 90% confluency by removing the medium, washing with DPBS (Gibco™, Thermo Fisher Scientific) and separating the cell with 2.5 ml of an Accutase® solution (Gibco™, Thermo Fisher Scientific). Cells were then incubated for 5-10 min at room temperature until a visible detachment of the cells was observed. Accutase™ was subsequently inactivated by adding 7.5 ml pre-warmed DMEM including 10% FBS and all supplements. Cells were then transferred into a new flask at an appropriate density or counted and plated on 96-well, 48-well or 6-well format for plasmid transfection.

2.6 Plasmid Transfection

Cells were transfected with X-tremeGENE HP (Roche) according to the protocol of the manufacturer. DNA amounts were kept constant in all transient experiments to yield reproducible complex formation and comparable results. In 96-well plate experiments, a total amount of 100 ng of plasmid DNA was used, in 48-well plates, a total amount of 300 ng of plasmid DNA was used, and in 6-well plates, a total amount of 2.4 μg of plasmid DNA was used per well. Cells were plated one day before transfection (25,000 cells/well in 100 μl for 96-well plates, 75,000 cells/well in 500 μl for 48-well plates, 600,000 cells/well in 3 ml for 6-well plate). 24 h post-transfection, 100 μl fresh medium was added on 96-well transfection per well, 48 h post-transfection 100 μL medium was removed and replaced with fresh medium on 96-well transfections per well. For modulation of alternative splicing with 5-iodotubercidin (5-ITU) (Sigma-Aldrich), 24 hours post-transfection 5-ITU (in DMSO) were applied on the cells. Control cells received the same volume of DMSO.

2.7 Generation of Stable Cell Lines Via CRISPR/Cas9

To generate a stable cell line (HEK293T, N2a), plasmids expressing a mammalian codon-optimized Cas9 from S. pyogenes (SpyCas9) with a tandem C-terminal SV40 nuclear localization signal (SV40 NLS) (CBh hybrid RNA-polymerase II promoter-driven) and a single-guide-RNA (sgRNA/gRNA, human U6 RNA-polymerase III promoter-driven) with a 19-21 bp cloned spacer targeting the exon-of-interest were used (for NEAT1, SEQ ID NO: 29). Notably, U6 promoter driven sgRNAs need a G for correct transcription start. If a target sgRNA does not contain a 5′-g, an extra g has to be added upstream the 20 nt spacer. Thus, 20×N for spacers containing a 5′-g. g+20N for spacers which does not contain a 5′-g can be used. The efficiency of CRISPR/Cas9 for a target site was performed by T7 endonuclease I assay (NEB) according to the manufacturer's protocol after 48-72 h post-transfection of cells with plasmids encoding Cas9 and the targeting sgRNA on a 48-well plate. Optionally, an i53 (SEQ ID NO: 11) expression plasmid (a genetically encoded 53bp1 inhibitor) was co-transfected to enhance homologous recombination (HR) after the Cas9-mediated double-strand break at the spacer-guided genomic site. Donor DNA plasmid contains the intein-flanked moiety including the selection-cassette to select for cells undergoing successful Cas9-mediated HR; moreover, the donor DNA plasmid contains homology arms of at least 800 bps flanking the to be inserted nucleic acid construct. 48 hours post-transfection (48-well or 6-well format), the medium was replaced with medium containing 50 μg/ml puromycin, if not otherwise indicated. Cells were observed daily and were detached with Accutase™ and re-plated with puromycin when surviving colonies reaches the colony size of about 50 cells. This step was repeated until no significant puromycin-mediated cell death could be observed. Those cells were plated without puromycin on 48-well plate and were transfected with a CAG-hybrid promoter-driven nuclear-localized FIp recombinase to excise the selection cassette. After one week, the cells were counter-selected with ganciclovir (2 and 10 μM) for another two weeks, before the cells were single-cell-sorted in 96-well plates and grown mono-clonally until colony size was big enough to be duplicated onto a second 96-well plate containing 2 μM ganciclovir. Cells which underwent successful cassette excision should survive ganciclovir treatment indicating and was a potential candidate for genotyping for zygosity. Those clones were detached and expanded on 48-well plates until confluency and half of the cell mass were then used subsequently for isolation of genomic DNA using Wizard® Genomic DNA Purification Kit (Promega). Genotyping of the genomic DNA was performed using LongAmp® Hot Start Taq 2× Master Mix (NEB) according to manufacturer's protocol with primer deoxynucleotides pairs (IDT) with at least one primer binding outside of the homology arms. The PCR product from clones, where the genotyping indicates homozygosity, were sent for Sanger-sequencing to verify its sequence integrity. NEAT1 was genotyped with following primers: SEQ ID NO: 30 and SEQ ID NO: 31. The reporter integrated KO-switch status was genotyped with: SEQ ID NO: 32 and SEQ ID NO: 33.

2.8 RNA-Analysis

2.8.1 Single-Molecule Fluorescence In-Situ Hybridization smFISH of NEAT1

HEK293T or its derived reporter clones were plated on 2-well p-slides (Ibidi) 24 hours before fixation (300,000 in 1.2 ml medium). Before fixation, cells were washed with DPBS (Gibco™, Thermo Fisher Scientific) and fixed for 10 min in 10% neutral buffered formalin (Sigma-Aldrich). After further three DPBS washing steps a 5 min, the cells were permeabilized for either overnight hours at 4° C. with ice-cold 70% ethanol or at RT for 1 hour. After three DPBS washing steps a 5 min, the samples were then incubated for 15 minutes with hybridization buffer prepared with 2× saline sodium citrate (SSC) solution+10% deionized formamide (Calbiochem®, Merck). Hybridization with Stellaris FISH probes was carried out in a total volume of 50 μl hybridization buffer containing 50 μg competitor tRNA from E. coli (Roche), 10% dextran sulfate (9011-18-1, VWR), 2 mg/ml UltraPure BSA (Thermo Fisher Scientific) and 10 mM ribonucleoside vanadyl complex (NEB) with probes in a final concentration of 1 ng/μl The preparations were covered with parafilm and incubated at 37° C. for at least 5 hours or overnight, and then washed twice with 37° C. preheated 2×SCC+10% deionized formamide at 37° C. for 30 minutes. Finally, the preparations were washed twice with DPBS at RT and then mounted with 10 μl ProLong Glass Antifade Mountant with NucBlue Stain (Thermo Fisher Scientific). The probes were pre-designed by Biosearch Technologies and supplied by the same. The probes included were human NEAT1 middle segment conjugated to Quasar570® (SMF-2037-1, Biosearch Technologies) and human NEAT1 5′-segment conjugated to Quasar670® (VSMF-2247-5). The automated quantification of the hybridization signal was performed with ImageJ (Fiji) software including the BioVoxxel toolbox plug-in.

2.8.2 Bioluminescence Quantification

For bioluminescence detection of secreted NLuc, the supernatant was collected (10 μL) 2 days post-seeding on 2-well p-slides (Ibidi) with 300,000 cells in 1.2 ml and detected using the Nano-Glo® Luciferase Assay System (Promega) on the Centro LB 960 (Berthold Technologies) plate reader with 0.5 s acquisition time.

### Example 2

Example 2 was carried out as shown in FIGS. 8-15 and accompanying figure legends herein.

### REFERENCES

1. Adriaens, C. et al. p53 induces formation of NEAT1 lncRNA-containing paraspeckles that modulate replication stress response and chemosensitivity. Nat. Med. 22, 861-868 (2016).
2. Araki, K., Araki, M. & Yamamura, K.-I. Site-directed integration of the cre gene mediated by Cre recombinase using a combination of mutant lox sites. Nucleic Acids Res. 30, e103 (2002).
3. Balzarini, J. et al. Engineering of a single conserved amino acid residue of herpes simplex virus type 1 thymidine kinase allows a predominant shift from pyrimidine to purine nucleoside phosphorylation. J. Biol. Chem. 281, 19273-19279 (2006).
4. Bao G, Rhee W J, Tsourkas A: Fluorescent probes for live-cell RNA detection. Annu Rev Biomed Eng, 2009; 11:25-47. doi: 10.1146/annurev-bioeng-061008-124920.
5. Beeharry, Y, Goodrum, G., Imperiale, C. J. & Pelchat, M. The Hepatitis Delta Virus accumulation requires paraspeckle components and affects NEAT1 level and PSP1 localization. Sci. Rep. 8, 6031 (2018).
6. Beyer, A. L. & Osheim, Y. N. Splice site selection, rate of splicing, and alternative splicing on nascent transcripts. Genes Dev. 2, 754-765 (1988).
7. Bochkov, Y. A. & Palmenberg, A. C. Translational efficiency of EMCV IRES in bicistronic vectors is dependent upon IRES sequence and gene location. Biotechniques 41, 283-4, 286, 288 passim (2006).
8. Braun, I. C., Rohrbach, E., Schmitt, C. & Izaurralde, E. TAP binds to the constitutive transport element (CTE) through a novel RNA-binding motif that is sufficient to promote CTE-dependent RNA export from the nucleus. EMBO J. 18, 1953-1965 (1999).
9. Carmody, S. R. & Wente, S. R. mRNA nuclear export at a glance. J. Cell Sci. 122, 1933-1937 (2009).
10. Carmo-Fonseca, M. & Kirchhausen, T. The timing of pre-mRNA splicing visualized in real-time. Nucleus 5, 11-14 (2014).
11. Carswell, S. & Alwine, J. C. Efficiency of utilization of the simian virus 40 late polyadenylation site: effects of upstream sequences. Mol. Cell. Biol. 9, 4248-4258 (1989).
12. Chamond, N., Deforges, J., Ulryck, N. & Sargueil, B. 40S recruitment in the absence of eIF4G/4A by EMCV IRES refines the model for translation initiation on the archetype of Type II IRESs. Nucleic Acids Res. 42, 10373-10384 (2014).
13. Choudhry, H. et al. Tumor hypoxia induces nuclear paraspeckle formation through HIF-2a dependent transcriptional activation of NEAT1 leading to cancer cell survival. Oncogene 34, 4546 (2015).
14. Chung Y, Klimanskaya I, Becker S, Li T, Maserati M, Lu S, Zdravkovic T, Ilic D, Genbacev O, Fisher S, Krtolica A, and Lanza R: Human Embryonic Stem Cell Lines Generated without Embryo Destruction. Cell Stem Cell, 2008; 2(2); 113-117.
15. Cong L, Ran F A, Cox D, Lin S, Barretto R, Habib N, Hsu P D, Wu X, Jiang W, Marraffini L A, and Zhang F: Multiplex Genome Engineering Using CRISPR/Cas Systems. Science, 2013; 15; 339(6121): 819-823.
16. Cullen, B. R. Nuclear mRNA export: insights from virology. Trends Biochem. Sci. 28, 419-424 (2003).
17. Darrouzet, E., Lindenthal, S., Marcellin, D., Pellequer, J.-L. & Pourcher, T. The sodium/iodide symporter: state of the art of its molecular characterization. Biochim. Biophys. Acta 1838, 244-253 (2014).
18. Donello, J. E., Loeb, J. E. & Hope, T. J. Woodchuck hepatitis virus contains a tripartite posttranscriptional regulatory element. J. Virol. 72, 5085-5092 (1998).
19. Gaj T, Gersbach C A, and Barbas C F: ZFN, TALEN and CRISPR/Cas-based methods for genome engineering. Trends Biotechnol, 2013; 31(7): 397-405 doi: 10.1016/j.tibtech.2013.04.004
20. Houseley, J. & Tollervey, D. The many pathways of RNA degradation. Cell 136, 763-776 (2009).
21. Imamura, K. et al. Long noncoding RNA NEAT1-dependent SFPQ relocation from promoter region to paraspeckle mediates IL8 expression upon immune stimuli. Mol. Cell 53, 393-406 (2014).
22. Kessler, M. M., Beckendorf, R. C., Westhafer, M. A. & Nordstrom, J. L. Requirement of A-A-U-A-A-A and adjacent downstream sequences for SV40 early polyadenylation. Nucleic Acids Res. 14, 4939-4952 (1986).
23. Kozak, M. How do eucaryotic ribosomes select initiation regions in messenger RNA? Cell 15, 1109-1123 (1978).
24. Kozak, M. The scanning model for translation: an update. J. Cell Biol. 108, 229-241 (1989).
25. Lander E S et al.: Initial sequencing and analysis of the human genome. Nature, 2001; 15; 409(6822):860-921.
26. Lanoix, J. & Acheson, N. H. A rabbit beta-globin polyadenylation signal directs efficient termination of transcription of polyomavirus DNA. EMBO J. 7, 2515-2522 (1988).
27. Le Hir, H., Gatfield, D., Izaurralde, E. & Moore, M. J. The exon-exon junction complex provides a binding platform for factors involved in mRNA export and nonsense-mediated mRNA decay. EMBO J. 20, 4987-4997 (2001).
28. Lellahi, S. M. et al. The long noncoding RNA NEAT1 and nuclear paraspeckles are up-regulated by the transcription factor HSF1 in the heat shock response. J. Biol. Chem. 293, 18965-18976 (2018).
29. Leppek, K., Das, R. & Barna, M. Functional 5′ UTR mRNA structures in eukaryotic translation regulation and how to find them. Nat. Rev. Mol. Cell Biol. 19, 158-174 (2018).
30. Levitt, N., Briggs, D., Gil, A. & Proudfoot, N. J. Definition of an efficient synthetic poly(A) site. Genes Dev. 3, 1019-1025 (1989).
31. Lv, J. et al. A Novel Ideal Radionuclide Imaging System for Non-invasively Cell Monitoring built on Baculovirus Backbone by Introducing Sleeping Beauty Transposon. Sci. Rep. 7, 43879 (2017).
32. Ma, H. et al. The long noncoding RNA NEAT1 exerts antihantaviral effects by acting as positive feedback for RIG-I signaling. Journal of (2017).
33. Miller W A, Wang Z, and Treder K: The amazing diversity of cap-independent translation elements in the 3′-untranslated regions of plant viral RNAs. Biochem Soc Trans, 2007; 35(Pt 6): 1629-1633.
34. Modic, M. et al. Cross-Regulation between TDP-43 and Paraspeckles Promotes Pluripotency-Differentiation Transition. Mol. Cell 74, 951-965.e13 (2019).
35. Oh, T., Bajwa, A., Jia, G. & Park, F. Lentiviral vector design using alternative RNA export elements. Retrovirology 4, 38 (2007).
36. Pan Q, Shai O, Lee L J, Frey B J, Blencowe B J.: Deep surveying of alternative splicing complexity in the human transcriptome by high-throughput sequencing. Nat Genet, 2008; 40(12):1413-5. doi: 10.1038/ng.259.
37. Pasquinelli, A. E. et al. The constitutive transport element (CTE) of Mason-Pfizer monkey virus (MPMV) accesses a cellular mRNA export pathway. EMBO J. 16, 7500-7510 (1997).
38. Penheiter, A. R., Russell, S. J. & Carlson, S. K. The Sodium Iodide Symporter (NIS) as an Imaging Reporter for Gene, Viral, and Cell-based Therapies. Current Gene Therapy 12, 33-47 (2012).
39. Pollard, V. W. & Malim, M. H. The HIV-1 Rev protein. Annu. Rev. Microbiol. 52, 491-532 (1998).
40. Popa, I., Harris, M. E., Donello, J. E. & Hope, T. J. CRM1-dependent function of a cis-acting RNA export element. Mol. Cell. Biol. 22, 2057-2067 (2002).
41. Rees H. A., Yeh W., and Liu D. R. Development of hRad51-Cas9 nickase fusions that mediate HDR without double strand breaks. Nat Comm, 2019 10: 2212: 1-12.
42. Schmohl, K. A. et al. Imaging and targeted therapy of pancreatic ductal adenocarcinoma using the theranostic sodium iodide symporter (NIS) gene. Oncotarget 8, 33393-33404 (2017).
43. Schnutgen, F. et al. A directional strategy for monitoring Cre-mediated recombination at the cellular level in the mouse. Nat. Biotechnol. 21, 562-565 (2003).
44. Shatsky, I. N., Dmitriev, S. E., Terenin, I. M. & Andreev, D. E. Cap- and IRES-independent scanning mechanism of translation initiation as an alternative to the concept of cellular IRESs. Mol. Cells 30, 285-293 (2010).
45. Sojka, D. K., Bruniquel, D., Schwartz, R. H. & Singh, N. J. IL-2 secretion by CD4+ T cells in vivo is rapid, transient, and influenced by TCR-specific competition. J. Immunol. 172, 6136-6143 (2004).
46. Stern, B., Olsen, L. C., Tröße, C., Ravneberg, H. & Pryme, I. F. Improving mammalian cell factories: The selection of signal peptide has a major impact on recombinant protein synthesis and secretion in mammalian cells. Trends Cell Mol. Biol. 2, 1-17 (2007).
47. Takata, Y, Kondo, S., Goda, N., Kanegae, Y. & Saito, I. Comparison of efficiency between FLPe and Cre for recombinase-mediated cassette exchange in vitro and in adenovirus vector production: RMCE efficiency of FLPe and Cre. Genes Cells 16, 765-777 (2011).
48. Teplova, M., Wohlbold, L., Khin, N. W., Izaurralde, E. & Patel, D. J. Structure-function studies of nucleocytoplasmic transport of retroviral genomic RNA by mRNA export factor TAP. Nat. Struct. Mol. Biol. 18, 990-998 (2011).
49. Tomek, W. & Wollenhaupt, K. The ‘closed loop model’ in controlling mRNA translation during development. Anim. Reprod. Sci. 134, 2-8 (2012).
50. Trösse, C., Ravneberg, H., Stern, B. & Pryme, I. F. Vectors encoding seven oikosin signal peptides transfected into CHO cells differ greatly in mediating Gaussia luciferase and human endostatin production although mRNA levels are largely unaffected. Gene Regul. Syst. Bio. 1, 303-312 (2007).
51. Venter J C et al.: The sequence of the human genome. Science, 2001; 16; 291(5507):1304-51.
52. Vicens, Q., Kieft, J. S. & Rissland, O. S. Revisiting the Closed-Loop Model and the Nature of mRNA 5′-3′ Communication. Mol. Cell 72, 805-812 (2018).
53. Wang E T, Sandberg R, Luo S, Khrebtukova I, Zhang L, Mayr C, Kingsmore S F, Schroth G P, and Burge C B: Alternative Isoform Regulation in Human Tissue Transcriptomes. Nature, 2008; 27; 456(7221): 470-476.
54. Wolff, J., Chaikoff, I. L., Goldberg, R. C. & Meier, J. R. THE TEMPORARY NATURE OF THE INHIBITORY ACTION OF EXCESS IODIDE ON ORGANIC IODINE SYNTHESIS IN THE NORMAL THYROID. Endocrinology 45, 504-513 (1949).
55. Yamazaki, T. et al. Functional Domains of NEAT1 Architectural lncRNA Induce Paraspeckle Assembly through Phase Separation. Mol. Cell 70, 1038-1053.e7 (2018).
56. Zhang, Q., Chen, C.-Y, Yedavalli, V. S. R. K. & Jeang, K.-T. NEAT1 long noncoding RNA and paraspeckle bodies modulate HIV-1 posttranscriptional expression. MBio 4, e00596-12 (2013).
57. Zhou, X., Li, B., Wang, J., Yin, H. & Zhang, Y. The feasibility of using a baculovirus vector to deliver the sodium-iodide symporter gene as a reporter. Nucl. Med. Biol. 37, 299-308 (2010).

1. A method for detecting a nucleic acid construct or part thereof and/or detecting the expression product of the nucleic acid construct or part thereof,
wherein the method comprises inserting a nucleic acid construct or part thereof into an intron or a synthetic intron,
wherein the nucleic acid construct comprises:
a. at least one heterologous nucleic acid sequence, which does not encode a protein;
at least one nucleic acid sequence for transcription of the nucleic acid construct or part thereof, and
at least one nucleic acid sequence for exporting the nucleic acid construct out of the nucleus,
or
b. at least one heterologous nucleic acid sequence, which encodes a protein,
at least one nucleic acid sequence for transcription of the nucleic acid construct or part thereof,
at least one nucleic acid sequence for preventing degradation of the nucleic acid construct or part thereof,
at least one nucleic acid sequence for exporting the nucleic acid construct out of the nucleus or part thereof, and
at least one nucleic acid sequence for translation of the nucleic acid construct or part thereof.

2. Method according to claim 1b, wherein the at least one nucleic acid sequence for translation of the nucleic acid construct or part thereof is a nucleic acid sequence for translation of the heterologous nucleic acid sequence.

3. Method according to claim 1 or 2, wherein the nucleic acid construct or part thereof is under the control of an endogenous promoter of the gene comprising the expression product of the nucleic acid construct or part thereof.

4. Method according to any one of the previous claims, wherein the at least one nucleic acid sequence for transcription of the nucleic acid construct or parts thereof comprises a splice donor nucleic acid sequence and a splice acceptor nucleic acid sequence; preferably wherein the splice donor nucleic acid sequence comprises or consists of SEQ ID NO: 1 and/or wherein the splice acceptor nucleic acid sequence comprises or consists of SEQ ID NO: 2.

5. Method according to any one of the previous claims, wherein the at least one nucleic acid sequence for exporting the nucleic acid construct or part thereof out of the nucleus is a viral sequence, preferably wherein the at least one nucleic acid sequence for exporting the nucleic acid construct or part thereof out of the nucleus comprises or consists of CTE according to SEQ ID NO: 3 and/or comprises or consists of WPRE according to SEQ ID NO: 4.

6. Method according to any one of the previous claims 1b and 2 to 4, wherein the at least one nucleic acid sequence for translation of the nucleic acid construct or part thereof is for translation of the heterologous nucleic acid sequence and is initiated by an internal ribosomal entry site (IRES); preferably wherein the at least one nucleic acid sequence for translation of the nucleic acid construct or part thereof is the internal ribosomal entry site of the virus Encephalomyocarditis virus (EMCV) according to SEQ ID NO: 5 or the internal ribosomal entry site of the Hepatitis C virus (HCV) according to SEQ ID NO: 6; and an open reading frame (ORF).

7. Method according to any one of the previous claims 1b and 2 to 6, wherein the at least one nucleic acid sequence for preventing degradation of the nucleic acid construct or part thereof is a poly-A-tail, preferably a synthetic poly-A-tail, more preferably wherein the synthetic poly-A-tail comprises at least 30 adenosines, and even more preferred wherein the poly-A-tail comprises or consists of the sequence according to SEQ ID NO: 7.

8. Method according to any one of the previous claims 1b and 2 to 7, wherein the at least one nucleic acid sequence for preventing degradation of the nucleic acid construct or part thereof is a polyadenylation signal, preferably a late SV40 polyadenylation signal or a rabbit beta-globin polyadenylation signal, more preferably the late SV40 polyadenylation signal is mutated to be unidirectional.

9. Method according to claim 8, wherein the polyadenylation signals are integrated in the nucleic acid construct in antisense direction and are enclosed with loxP sites and wherein after transcription the inverted polyadenylation signal is not separated from the endogenous gene product.

10. Method according to claim 9, wherein after the transcription a Cre recombinase (SEQ ID NO: 8) is administered to the transcript to invert the polyadenylation signals into sense direction.

11. Method according to any one of the previous claims, wherein the method is non- or minimally invasive for the expression product of the intron or synthetic intron such that a native and/or fully functional protein is expressed compared to the protein without insertion of the nucleic acid construct or part thereof.

12. Method according to any one of the previous claims, comprising the insertion of the nucleic acid construct with targeted transgene insertion.

13. Method according to any one of the previous claims, wherein the at least one heterologous nucleic acid sequence encodes for a protein-coding RNA, a non-coding RNA, a miRNA, an aptamer, a siRNA, a synthetic RNA sequence or a barcode for extranuclear detection.

14. Method according to any one of the previous claims, wherein the at least one heterologous nucleic acid sequence is detected and enables to detect a specific cell.

15. Method according to any one of the previous claims, wherein the at least one heterologous nucleic acid sequence is detected and provides information about the transcriptional regulation of the cell or a time stamp of a cellular process.

16. Method according to any one of the previous claims, wherein the heterologous nucleic acid sequence encodes a protein or enzyme selected from the group consisting of a fluorescent protein, preferably green fluorescent protein; a bioluminescence-generating enzyme, preferably NanoLuc, NanoKAZ, TurboLuc, Cypridina, Firefly, Renilla luciferase, split luciferase, split APEX2 or mutant derivatives thereof; an enzyme, which is capable of generating a coloured pigment, preferably tyrosinase or an enzyme of a multi-enzymatic process, more preferably the violacein or betanidin synthesis process, a genetically encoded receptor for multimodal contrast agents, preferably Avidin, Streptavidin or HaloTag or mutant derivatives thereof; an enzyme, which is capable of converting a non-reporter molecule into a reporter molecule, preferably TEV protease and picornaviral proteases, more preferably rhinoviral 3C proteases and polioviral 3C protease, SUMO proteases and mutant derivatives thereof; an enzyme, which is capable of inactivating a toxic compound, preferably blasticidin-S-deaminase, puromycin-N-acetyltransferase, neomycin phosphotransferase, hygromycin B phosphotransferase and mutant derivatives thereof, an enzyme, which is capable of converting pro-drug/toxin-mediated toxicity, preferably thymidine kinase and mutant derivatives thereof and a small-molecule sensor protein, preferably calmodulin, troponin C, S100 and mutant derivatives thereof.

17. Method according to claim 15, wherein the method further comprises combining the expression of the protein or enzyme encoded by the heterologous nucleic acid sequence to the natural expression of the gene comprising the nucleic acid construct or part thereof by using the same promotor.

18. Method according to any one of the previous claims, wherein the heterologous nucleic acid sequence encodes a resistance gene for cell-toxic compounds, preferably wherein the method additionally comprises detecting the survival of the cells comprising the nucleic acid construct or part thereof, more preferably wherein the resistance gene for cell-toxic compounds is used as a selection marker of the cells comprising the nucleic acid construct or part thereof.

19. Method according to any one of the previous claims, wherein the heterologous nucleic acid sequence encodes a Cas enzyme selected from the group consisting of Cas9 (SEQ ID NO: 9), Cas12a, Cas12b, Cas12c, Cas13a, Cas13b, Cas13d, Cas14, CasX, and fusion proteins thereof.

20. Method according to any one of the previous claims, wherein the heterologous nucleic acid sequence encodes an amino acid, which can be metabolized to an antibiotic or derivative thereof, preferably for inducing a genetic system, more preferably for inducing the genetic Tet-On/Tet-OFF system.

21. Method according to any one of the previous claims, wherein the heterologous nucleic acid sequence encodes an enzyme of a biosynthesis pathway generating a toxin or a mutant thereof.

22. Method according to any one of the previous claims, wherein the heterologous nucleic acid sequence is a suicide gene or a gene, which induces a cell death cascade.

23. Method according to any one of the previous claims, wherein the heterologous nucleic acid sequence further comprises a polynucleotide encoding a protein, which functions as an activator of the expression of the gene comprising the nucleic acid construct or part thereof.

24. Method according to any one of the previous claims, wherein the heterologous nucleic acid sequence encodes a transcription factor.

25. Method according to claim 24, wherein the transcription factor is used to force or refine determination of a stem cell into a defined mature cell.

26. Method according to any one of the previous claims, wherein the heterologous nucleic acid sequence encodes a transcriptional regulator or a repressor protein or an intrabody.

27. Method according to any one of the previous claims, wherein the heterologous nucleic acid sequence encodes a protein, which is a hormone or has the function of a hormone.

28. Method according to any one of the previous claims, wherein the heterologous nucleic acid sequence encodes a protein, which is a receptor, preferably a hormone receptor or a mutant derivate thereof.

29. Method according to any one of the previous claims, wherein the heterologous nucleic acid sequence encodes an affinity domain or tag to bind protein, DNA or RNA.

30. Method according to claim 29, wherein the protein affinity domain is used to capture the expression product of the nucleic acid construct or part thereof, preferably the expression product of the heterologous nucleic acid sequence.

31. Method according to any one of the previous claims, wherein the heterologous nucleic acid sequence encodes an antibody or antibody fragment.

32. Method according to claim 31, wherein the antibody or antibody fragment is used to capture the expression product of the nucleic acid construct or part thereof, preferably the expression product of the heterologous nucleic acid sequence.

33. Method according to any one of the previous claims, wherein the protein or enzyme encoded by the heterologous nucleic acid sequence is for preventing pathological changes within the cell.

34. Method according to any one of the previous claims, for detecting biological functions, preferably the regulation of tissue and cell generation, more preferably neuroregeneration.

35. Nucleic acid construct comprising or consisting of any of SEQ ID NOs: 1 to 7.

36. Nucleic acid construct according to claim 35, for use in therapy.

37. Nucleic acid construct according to claim 35, for use in the treatment or prevention of cancer.

38. A vector comprising any nucleic acid construct of claim 35.

39. A cell comprising any nucleic acid construct of claim 35 or the vector of claim 38.

40. Use of any nucleic acid construct of claim 35, the vector of claim 38 or the cell of claim 39 for detecting the cell identity, the cell state or the time point of expression of the nucleic acid construct.

41. Use of any nucleic acid construct of claim 35, the vector of claim 38 or the cell of claim 39 for enriching cells.

42. The nucleic acid construct of claim 35, the vector of claim 38 or the cell of claim 39 for use in the treatment or prevention of a disease, preferably wherein the disease is selected from the group consisting of retinopathies, tauopathies, motor neuron diseases, muscular diseases, neurodevelopmental and neurodegenerative diseases, more preferably selected from the group consisting of cystic fibrosis, retinitis pigmentosa, myotonic dystrophy, Alzheimer's disease and Parkinson's disease.

43. The nucleic acid construct of claim 35, the vector of claim 38 or the cell of claim 39 for use in tissue generation, gene therapy and in vitro reprogramming of cells.

44. The nucleic acid construct of claim 35, the vector of claim 38 or the cell of claim 39 for use as a medicament.

45. Use of any nucleic acid construct of claim 35, the vector of claim 38 or the cell of claim 40 in tissue engineering.

46. Kit for detecting a nucleic acid construct or part thereof and/or detecting the expression product of the nucleic acid construct or part thereof,
wherein the kit comprises:
a first vector comprising the nucleic acid construct or part thereof, which comprises
a. at least one heterologous nucleic acid sequence, which does not encode a protein;
at least one nucleic acid sequence for transcription of the nucleic acid construct or part thereof, and
at least one nucleic acid sequence for exporting the nucleic acid construct out of the nucleus,
or
b. at least one heterologous nucleic acid sequence, which encodes a protein,
at least one nucleic acid sequence for transcription of the nucleic acid construct or part thereof,
at least one nucleic acid sequence for translation of the nucleic acid construct or part thereof,
at least one nucleic acid sequence for preventing degradation of the nucleic acid construct or part thereof, and
at least one nucleic acid sequence for exporting the nucleic acid construct out of the nucleus or part thereof, and
a second vector coding for a guided endonuclease, preferably wherein the endonuclease is selected from the group consisting of Cas9 (SEQ ID NO: 9), Cas12a, TALENs, ZFNs and meganucleases.

47. Kit according to claim 46, wherein the at least one nucleic acid sequence for transcription of the nucleic acid construct or part thereof comprises a splice donor nucleic acid sequence and a splice acceptor nucleic acid sequence; preferably wherein the splice donor nucleic acid sequence comprises or consists of SEQ ID NO: 1 and/or wherein the splice acceptor nucleic acid sequence comprises or consists of SEQ ID NO: 2.

48. Kit according to claim 46 or 47, wherein the at least one nucleic acid sequence for exporting the nucleic acid construct or part thereof out of the nucleus is a viral sequence, preferably comprises or consists of CTE according to SEQ ID NO: 3 and/or comprises or consists of WPRE according to SEQ ID NO: 4.

49. Kit according to any one of claims 46 to 48, wherein the first plasmid further comprises an internal ribosomal entry site (IRES), wherein the at least one nucleic acid sequence for translation of the nucleic acid construct or part thereof is for translation of the heterologous nucleic acid sequence and is initiated by an internal ribosomal entry site (IRES); preferably the internal ribosomal entry site of the virus Encephalomyocarditis virus (EMCV) according to SEQ ID NO: 5 or the internal ribosomal entry site of the Hepatitis C virus (HCV) according to SEQ ID NO: 6; and an open reading frame (ORF).

50. Kit according to any one of claims 46 to 49, wherein the at least one nucleic acid sequence for preventing degradation of the nucleic acid construct or part thereof is a poly-A-tail, preferably a synthetic poly-A-tail, more preferably wherein the synthetic poly-A-tail comprises at least 30 adenosines, and even more preferred, wherein the poly-A-tail comprises or consists of the sequence according to SEQ ID NO: 7.

51. Kit according to any one of claims 46 to 50, the heterologous nucleic acid sequence encodes a protein or enzyme selected from the group consisting of a fluorescent protein, preferably green fluorescent protein; a bioluminescence-generating enzyme, preferably NanoLuc, NanoKAZ, TurboLuc, Cypridina, Firefly, Renilla luciferase, split luciferase, split APEX2 or mutant derivatives thereof; an enzyme, which is capable of generating a coloured pigment, preferably tyrosinase or an enzyme of a multi-enzymatic process, more preferably the violacein or betanidin synthesis process, a genetically encoded receptor for multimodal contrast agents, preferably Avidin, Streptavidin or HaloTag or mutant derivatives thereof; an enzyme, which is capable of converting a non-reporter molecule into a reporter molecule, preferably TEV protease, SUMO proteases and mutant derivatives thereof; an enzyme, which is capable of inactivating a toxic compound, preferably blasticidin-S-deaminase, puromycin-N-acetyltransferase, neomycin phosphotransferase, hygromycin B phosphotransferase and mutant derivatives thereof, an enzyme, which is capable of converting pro-drug/toxin-mediated toxicity, preferably thymidine kinase and mutant derivatives thereof and a small-molecule sensor protein, preferably calmodulin, troponin C, S100 and mutant derivatives thereof.


SEQUENCE LISTING (54 sequences)

SEQ ID NO: 1 - DNA, 67 bp, Artificial Sequence
  a splice donor (SD)
gtaagtatcctttttacagcacaacttaatgagacagatagaaactggtcttgtagaaacagagtag

SEQ ID NO: 2 - DNA, 60 bp, Artificial Sequence
  a splice acceptor (SA)
tcgcctgcttttctgcctggtactaacttctctcccctctcctcttttctttttctgcag

SEQ ID NO: 3 - DNA, 170 bp, Artificial Sequence
  CTE (constitutive transport element) from Simian Mason-Pfizer D-type retrovirus (MPMV/6A)
acctcccctgtgagctagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgt

SEQ ID NO: 4 - DNA, 590 bp, Artificial Sequence
  WPRE (woodchuck hepatitis virus post- transcriptional response element) with mutations to inactivate potential start site for a cancerogenic X-protein and a compensating mutation to prevent secondary structure change
taatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccaaggctgctcgcctgtgttgcctcctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 5 - DNA, 588 bp, Artificial Sequence
  Internal ribosomal entry site (IRES) from encephalomyocarditis virus (EMCV)
cccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccaccacc

SEQ ID NO: 6 - DNA, 341 bp, Artificial Sequence
  Internal ribosome entry site (IRES) from Hepatitis C virus (HCV)
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcacc

SEQ ID NO: 7 - DNA, 50 bp, Artificial Sequence
  A-homopolymer
aaaaaaaaaaaaaaaaaaaaaaaaaaaaaaaaaaaaaaaaaaaaaaaaaa

SEQ ID NO: 8 - protein, 356 aa, Artificial Sequence
  Cre recombinase with C-terminal c-Myc NLS
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGDGGSGPAAKRVKLD

SEQ ID NO: 9 - protein, 1401 aa, Artificial Sequence
  Streptococcus pyogenes Cas9 amino acid sequence with C-terminal tandem SV40 NLS and HA epitope tag
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVD

SEQ ID NO: 10 - protein, 440 aa, Artificial Sequence
  Flp recombinase with C-terminal c-Myc NLS
MSQFDILCKTPPKVLVRQFVERFERPSGEKIASCAAELTYLCWMITHNGTAIKRATFMSYNTIISNSLSFDIVNKSLQFKYKTQKATILEASLKKLIPAWEFTIIPYNGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYPIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRIGGSGGSPAAKRVKLDSG

SEQ ID NO: 11 - protein, 74 aa, Artificial Sequence
  i53, the inhibitor of 53BP1.
MLIFVKTLTGKTITLEVEPSDTIENVKAKIQDKEGIPPDQQRLAFAGKSLEDGRTLSDYNILKDSKLHPLLRLR

SEQ ID NO: 12 - DNA, 787 bp, Artificial Sequence
  loxP-WT_loxP-2272_synthetic-pA-rv_SV40-late-pA- mut-rv_rabbit-beta-globin-pA-mut-rv_rabbit-beta-globin-2nd-intron- SA-rv_loxP-WT-rv_rabbit-beta-globin-2nd-intron-SD-rv _loxP-2272-rv
  misc_feature (1)..(34): loxP-WT
  misc_feature (105)..(138): loxP-2272
  misc_feature (170)..(217): synthetic-pA-rv
  misc_feature (226)..(446): SV40-late-pA-mut-rv
  misc_feature (453)..(560): rabbit-beta-globin-pA-mut-rv
  misc_feature (561)..(630): rabbit-beta-globin-2nd-intron-SA-rv
  misc_feature (650)..(682): loxP-2272-rv
  misc_feature (697)..(739): rabbit-beta-globin-2nd-intron-SD-rv
  misc_feature (754)..(787): loxP-WT-rv
ataacttcgtataatgtatgctatacgaagttatgggtcgatgctgagatctggactagagggtcgatggtgatgcttggatcaaaataggaagaatcccaatgataacttcgtataaagtatcctatacgaagttatactagaattgcgtaccgccaccatgtcgcgacacacaaaaaaccaacacacagatctctagagctctggatcttttattgcccgggctaccacatttgtagaggttttacttgctttaaaaaacctcccacacctccccctgaacctgaaacataaaatgaatgcaattgttgttgttaacttgtttattgcagcttataatggttacaaatatagcaatagcatcacaaatttcacaaatatagcatttttttcactgcattctagttgtggtttgtccaaactcatcaatgtatcttatcatgtctgtatactttaaatgatttgccctcccatatgtccttccgagtgagagacacaaaaaattccaacacactattgcaatgaaaatacatttcctttattagccagaagtcagatgccagcacaacaaccagcacgttgcccaggagctgtaggaaaaagaagaaggcatgaacatagttagcagagttacgtagcgatcgctagtataacttcgtatagcatacattatacgaagttatgggtcgatggtgacaatagcgaaaaagaaagaacaatcaagggtccccaaactcacctaggtcctttgatataacttcgtataggatactttatacgaagttat

SEQ ID NO: 13 - DNA, 6946 bp, Artificial Sequence
  Intron-encoded secretory-NLuc with synthetic SD (splice donor) and SA (splice acceptor) and with a reporter, F3-sites-flanked-EF1a-Puro-2A-HSV-TK-cassette
  misc_feature (899)..(4035): F3-sites-flanked-EF1a-Puro-2A-HSV-TK-cassette
  CDS (4658)..(5230): secretory-NLuc
gtaagtatcctttttacagcacaacttaatgagacagatagaaactggtcttgtagaaacagagtagattttatcgaattgcaacctcttcgagggacataacttcgtataatgtatgctatacgaagttatgggtcgatgctgagatctggactagagggtcgatggtgatgcttggatcaaaataggaagaatcccaatgataacttcgtataaagtatcctatacgaagttatactagaattgcgtaccgccaccatgtcgcgacacacaaaaaaccaacacacagatctctagagctctggatcttttattgcccgggctaccacatttgtagaggttttacttgctttaaaaaacctcccacacctccccctgaacctgaaacataaaatgaatgcaattgttgttgttaacttgtttattgcagcttataatggttacaaatatagcaatagcatcacaaatttcacaaatatagcatttttttcactgcattctagttgtggtttgtccaaactcatcaatgtatcttatcatgtctgtatactttaaatgatttgccctcccatatgtccttccgagtgagagacacaaaaaattccaacacactattgcaatgaaaatacatttcctttattagccagaagtcagatgccagcacaacaaccagcacgttgcccaggagctgtaggaaaaagaagaaggcatgaacatagttagcagagttacgtagcgatcgctagtataacttcgtatagcatacattatacgaagttatgggtcgatggtgacaatagcgaaaaagaaagaacaatcaagggtccccaaactcacctaggtcctttgatataacttcgtataggatactttatacgaagttatggcatctccttcggaagttcctattctatttgaagtataggaacttccaggtccaggattttcctcgacgtcccctgcctgcttgagcagtgaaaagttagttgccccggatccgttggcttcgcccatctctctagcgaaggttctggcgaggtcacagatggtggggatgctgccaggggttgtcacatgggtctgcaccatgccgcttgtcagttgcagcagggcatctctgcatccggctgggctctgatcgtagtccaggatgaacacatgcatgggccgcagtctttttgccagcacgtcgagagcccaggcgaacacgttatacaggtcgccgttaggggccagcagttcaggggctctgaacagtgtgaacagggtgtcgccgatgtgaggtctgggtccagcgttagactgaggctcagcgccttgtggaggaactgctgtgccagagagctgtccccagtcctctctccaggaaccgccgccttgaagatagcgcacggtgttagccagcaggccgtacactcttctgatggcggccagcatagccagatccagtctctcgccaggcctctgtctcttagccagtctgtcgatgtgtctgtcctctggcagggctccaagcacgatgtttgtgccgggcagtgtaggaggaatcagagccacgaaagccagcacggcctgaggtgtcatgctgcccatcagataccgagcggcagggtaacacagcagggcgaagatggggtgtctgtcgaagatcagggtcagagcaggtggtggagcgtgagaagatccagcctcgccgccaatatgtggagccagcacagcgtctgtcacggcgtaaggcatgcccattgtgatctgggcagatgtcatgaccacagcagcgtcgcctgcgctaatttcgccctggtccagtctgtgctgtgtggtgtagatgttggcgattgtctcagaggctcccaggacctgccagtaggtcataggctcgggcacgtacacgatgtcatctctgctgcccagagccaccagcagctgtgttgttgtggtcttgcccatgccatgagggccgtcgatatacactctcagcagtgtgggcatcttctgttccagccgcacttctgtagcctcttgctgccttcttggcctgagggctgttcttctgttgctgtggcctctgcttctagcggcttgatcgaaggcgctggcgtgctggtgacaaggataagaagcaggaccggggttctcttccacgtcgccagcctgtttcagcaggctgaagtttgtagcgccggatccggcgccaggctttctggtcatgcaccaggttctagggccctcaggcacttccacgtcggcggtcacggtgaagcccagtctctcgtagaagggcaggtttctgggggcgcttgtttccaggaaggcgggcacgccagccctttcagcagcttccaccccaggcagcaccacagcagatcccagtcccttgccctggtggtcaggtgacacgcccacggtggccagaaaccaggcaggctcttttggtctgtggggggccagcaggccttccatctgctgctgggcagccagtctagagccgctcagctcggccattctaggtccgatctcggcgaacacagcgccggcttccacagactcaggggttgtccacacagccacagcggcgccatcatcggccacccacactttgccgatgtccaggcccactctggtcagaaacagttcctgcagctcggtcactctctcgatgtgccggtcggggtccacggtgtgtcttgtggcagggtaatcggcgaaggcagcggccagtgtccgcacagctcttggcacatcgtccctggtggccagccgcactgtgggcttgtactcggtcatggtggcgcgccttttaggggtagttttcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggggcgtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccaattcgaagttcctattctatttgaagtataggaacttcaaatacaagggcttactgtgcggactgggacgcccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccaccaccatggtggtcaaggtgctgttcgccctgatctgtattgccgtggccgaggccaagcctaccgtgttcaccctggaagatttcgtcggcgattggagacagaccgccggatacaacctggaccaggtgttggagcaaggcggcgtgtccagcctgtttcagaacctgggagtgtccgtgacacccatccagagaatcgtgctgagcggcgagaacggcctgaagatcgacatccatgtgatcatcccttacgagggcctgtccggcgatcagatgggccagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacggcaacaagatcatcgacgagcggctgatcaaccccgatggcagcctgctgttcagagtgaccatcaacggcgtgacaggatggcggctgtgcgaaagaatcctggcctaatgtacagatatcaggccacctcccctgtgagctagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgtcttttgttttttataattaaaaagggtgatggccaaggccacctcccctgtgagctagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgtcttttgttttttataattaaaaagggtgacacgtgaggccacctcccctgtgagctagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgtcttttgttttttataattaaaaagggtgaagcgctaggccacctcccctgtgagctagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgtcttttgttttttataattaaaaagggtgagtttaaactaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccaaggctgctcgcctgtgttgcctcctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttgcggccgcggcgcgtcttaaataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagcgattaagcggccgcctcgagtcgcctgcttttctgcctggtactaacttctctcccctctcctcttttctttttctgcag

SEQ ID NO: 14 - protein, 190 aa, Artificial Sequence
  Synthetic Construct
MVVKVLFALICIAVAEAKPTVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 15 - DNA, 34 bp, Artificial Sequence
  loxP-WT
ataacttcgtataatgtatgctatacgaagttat

SEQ ID NO: 16 - DNA, 34 bp, Artificial Sequence
  loxP-2272
ataacttcgtataaagtatcctatacgaagttat

SEQ ID NO: 17 - DNA, 48 bp, Artificial Sequence
  synthetic-pA-rv
cacacaaaaaaccaacacacagatctctagagctctggatcttttatt

SEQ ID NO: 18 - DNA, 221 bp, Artificial Sequence
  SV40-late-pA-mut-rv
taccacatttgtagaggttttacttgctttaaaaaacctcccacacctccccctgaacctgaaacataaaatgaatgcaattgttgttgttaacttgtttattgcagcttataatggttacaaatatagcaatagcatcacaaatttcacaaatatagcatttttttcactgcattctagttgtggtttgtccaaactcatcaatgtatcttatcatgtct

SEQ ID NO: 19 - DNA, 108 bp, Artificial Sequence
  rabbit-beta-globin-pA-mut-rv
tttaaatgatttgccctcccatatgtccttccgagtgagagacacaaaaaattccaacacactattgcaatgaaaatacatttcctttattagccagaagtcagatgc

SEQ ID NO: 20 - DNA, 70 bp, Artificial Sequence
  rabbit-beta-globin-2nd-intron-SA-rv
cagcacaacaaccagcacgttgcccaggagctgtaggaaaaagaagaaggcatgaacatagttagcagag

SEQ ID NO: 21 - DNA, 34 bp, Artificial Sequence
  loxP-2272-rv
ataacttcgtatagcatacattatacgaagttat

SEQ ID NO: 22 - DNA, 43 bp, Artificial Sequence
  rabbit-beta-globin-2nd-intron-SD-rv
caatagcgaaaaagaaagaacaatcaagggtccccaaactcac

SEQ ID NO: 23 - DNA, 34 bp, Artificial Sequence
  loxP-WT-rv
ataacttcgtataggatactttatacgaagttat

SEQ ID NO: 24 - DNA, 3137 bp, Artificial Sequence
  F3-sites-flanked-EF1a-Puro-2A-HSV-TK-cassette
gaagttcctattctatttgaagtataggaacttccaggtccaggattttcctcgacgtcccctgcctgcttgagcagtgaaaagttagttgccccggatccgttggcttcgcccatctctctagcgaaggttctggcgaggtcacagatggtggggatgctgccaggggttgtcacatgggtctgcaccatgccgcttgtcagttgcagcagggcatctctgcatccggctgggctctgatcgtagtccaggatgaacacatgcatgggccgcagtctttttgccagcacgtcgagagcccaggcgaacacgttatacaggtcgccgttaggggccagcagttcaggggctctgaacagtgtgaacagggtgtcgccgatgtgaggtctgggtccagcgttagactgaggctcagcgccttgtggaggaactgctgtgccagagagctgtccccagtcctctctccaggaaccgccgccttgaagatagcgcacggtgttagccagcaggccgtacactcttctgatggcggccagcatagccagatccagtctctcgccaggcctctgtctcttagccagtctgtcgatgtgtctgtcctctggcagggctccaagcacgatgtttgtgccgggcagtgtaggaggaatcagagccacgaaagccagcacggcctgaggtgtcatgctgcccatcagataccgagcggcagggtaacacagcagggcgaagatggggtgtctgtcgaagatcagggtcagagcaggtggtggagcgtgagaagatccagcctcgccgccaatatgtggagccagcacagcgtctgtcacggcgtaaggcatgcccattgtgatctgggcagatgtcatgaccacagcagcgtcgcctgcgctaatttcgccctggtccagtctgtgctgtgtggtgtagatgttggcgattgtctcagaggctcccaggacctgccagtaggtcataggctcgggcacgtacacgatgtcatctctgctgcccagagccaccagcagctgtgttgttgtggtcttgcccatgccatgagggccgtcgatatacactctcagcagtgtgggcatcttctgttccagccgcacttctgtagcctcttgctgccttcttggcctgagggctgttcttctgttgctgtggcctctgcttctagcggcttgatcgaaggcgctggcgtgctggtgacaaggataagaagcaggaccggggttctcttccacgtcgccagcctgtttcagcaggctgaagtttgtagcgccggatccggcgccaggctttctggtcatgcaccaggttctagggccctcaggcacttccacgtcggcggtcacggtgaagcccagtctctcgtagaagggcaggtttctgggggcgcttgtttccaggaaggcgggcacgccagccctttcagcagcttccaccccaggcagcaccacagcagatcccagtcccttgccctggtggtcaggtgacacgcccacggtggccagaaaccaggcaggctcttttggtctgtggggggccagcaggccttccatctgctgctgggcagccagtctagagccgctcagctcggccattctaggtccgatctcggcgaacacagcgccggcttccacagactcaggggttgtccacacagccacagcggcgccatcatcggccacccacactttgccgatgtccaggcccactctggtcagaaacagttcctgcagctcggtcactctctcgatgtgccggtcggggtccacggtgtgtcttgtggcagggtaatcggcgaaggcagcggccagtgtccgcacagctcttggcacatcgtccctggtggccagccgcactgtgggcttgtactcggtcatggtggcgcgccttttaggggtagttttcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggggcgtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccaattcgaagttcctattctatttgaagtataggaacttc

SEQ ID NO: 25 - DNA, 202 bp, Artificial Sequence
  CTE with additional nucleotides derived from Simian Mason-Pfizer D-type retrovirus (MPMV/6A)
gccacctcccctgtgagctagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgtcttttgttttttataattaaaaagggtga

SEQ ID NO: 26 - DNA, 113 bp, Artificial Sequence
  Chimeric fusion of crRNA and tracrRNA of S. pyogenes with mutations
  misc_feature (1)..(20): generic 20 nucleotides long spacer sequence, n=a or g or c or t
nnnnnnnnnnnnnnnnnnnngtttaagagctatgctggaaacagcatagcaagtttaaataaggctagtccgttatcaacttgaaaaagtggcaccgagtcggtgcttttttt

SEQ ID NO: 27 - DNA, 93 bp, Artificial Sequence
  Chimeric fusion of crRNA and tracrRNA of S. pyogenes with mutations
gtttaagagctatgctggaaacagcatagcaagtttaaataaggctagtccgttatcaacttgaaaaagtggcaccgagtcggtgcttttttt

SEQ ID NO: 28 - DNA, 103 bp, Streptococcus pyogenes
  misc_feature (1)..(20): generic 20 nucleotides long spacer sequence, n=a or g or c or t
nnnnnnnnnnnnnnnnnnnngttttagagctagaaatagcaagttaaaataaggctagtccgttatcaacttgaaaaagtggcaccgagtcggtgcttttttt

SEQ ID NO: 29 - DNA, 21 bp, Artificial Sequence
  NEAT1 spacer targeting the exon-of-interest
gcatctgaaaacctttacccc

SEQ ID NO: 30 - DNA, 23 bp, Artificial Sequence
  NEAT1 primer 1
caaaagttgtggcaagtccagcc

SEQ ID NO: 31 - DNA, 23 bp, Artificial Sequence
  NEAT1 primer 2
acgtgatcctgctcacacctttg

SEQ ID NO: 32 - DNA, 23 bp, Artificial Sequence
  reporter integrated KO-switch status primer 1
cagagttacgtagcgatcgctag

SEQ ID NO: 33 - DNA, 25 bp, Artificial Sequence
  reporter integrated KO-switch status primer 2
caaatagaataggaacttccgaagg

SEQ ID NO: 34 - protein, 1302 aa, Artificial Sequence
  Cas12c1 protein
MQTKKTHLHLISAKASRKYRRTIACLSDTAKKDLERRKQSGAADPAQELSCLKTIKFKLEVPEGSKLPSFDRISQIYNALETIEKGSLSYLLFALILSGFRIFPNSSAAKTFASSSCYKNDQFASQIKEIFGEMVKNFIPSELESILKKGRRKNNKDWTEENIKRVLNSEFGRKNSEGSSALFDSFLSKFSQELFRKFDSWNEVNKKYLEAAELLDSMLASYGPFDSVCKMIGDSDSRNSLPDKSTIAFTNNAEITVDIESSVMPYMAIAALLREYRQSKSKAAPVAYVQSHLTTTNGNGLSWFFKFGLDLIRKAPVSSKQSTSDGSKSLQELFSVPDDKLDGLKFIKEACEALPEASLLCGEKGELLGYQDFRTSFAGHIDSWVANYVNRLFELIELVNQLPESIKLPSILTQKNHNLVASLGLQEAEVSHSLELFEGLVKNVRQTLKKLAGIDISSSPNEQDIKEFYAFSDVLNRLGSIRNQIENAVQTAKKDKIDLESAIEWKEWKKLKKLPKLNGLGGGVPKQQELLDKALESVKQIRHYQRIDFERVIQWAVNEHCLETVPKFLVDAEKKKINKESSTDFAAKENAVRFLLEGIGAAARGKTDSVSKAAYNWFVVNNFLAKKDLNRYFINCQGCIYKPPYSKRRSLAFALRSDNKDTIEVVWEKFETFYKEISKEIEKFNIFSQEFQTFLHLENLRMKLLLRRIQKPIPAEIAFFSLPQEYYDSLPPNVAFLALNQEITPSEYITQFNLYSSFLNGNLILLRRSRSYLRAKFSWVGNSKLIYAAKEARLWKIPNAYWKSDEWKMILDSNVLVFDKAGNVLPAPTLKKVCEREGDLRLFYPLLRQLPHDWCYRNPFVKSVGREKNVIEVNKEGEPKVASALPGSLFRLIGPAPFKSLLDDCFFNPLDKDLRECMLIVDQEISQKVEAQKVEASLESCTYSIAVPIRYHLEEPKVSNQFENVLAIDQGEAGLAYAVFSLKSIGEAETKPIAVGTIRIPSIRRLIHSVSTYRKKKQRLQNFKQNYDSTAFIMRENVTGDVCAKIVGLMKEFNAFPVLEYDVKNLESGSRQLSAVYKAVNSHFLYFKEPGRDALRKQLWYGGDSWTIDGIEIVTRERKEDGKEGVEKIVPLKVFPGRSVSARFTSKTCSCCGRNVFDWLFTEKKAKTNKKFNVNSKGELTTADGVIQLFEADRSKGPKFYARRKERTPLTKPIAKGSYSLEEIERRVRTNLRRAPKSKQSRDTSQSQYFCVYKDCALHFSGMQADENAAINIGRRFLTALRKNRRSDFPSNVKISDRLLDN

SEQ ID NO: 35 - protein, 1218 aa, Artificial Sequence
  Cas12c2 protein
MTKHSIPLHAFRNSGADARKWKGRIALLAKRGKETMRTLQFPLEMSEPEAAAINTTPFAVAYNAIEGTGKGTLFDYWAKLHLAGFRFFPSGGAATIFRQQAVFEDASWNAAFCQQSGKDWPWLVPSKLYERFTKAPREVAKKDGSKKSIEFTQENVANESHVSLVGASITDKTPEDQKEFFLKMAGALAEKFDSWKSANEDRIVAMKVIDEFLKSEGLHLPSLENIAVKCSVETKPDNATVAWHDAPMSGVQNLAIGVFATCASRIDNIYDLNGGKLSKLIQESATTPNVTALSWLFGKGLEYFRTTDIDTIMQDFNIPASAKESIKPLVESAQAIPTMTVLGKKNYAPFRPNFGGKIDSWIANYASRLMLLNDILEQIEPGFELPQALLDNETLMSGIDMTGDELKELIEAVYAWVDAAKQGLATLLGRGGNVDDAVQTFEQFSAMMDTLNGTLNTISARYVRAVEMAGKDEARLEKLIECKFDIPKWCKSVPKLVGISGGLPKVEEEIKVMNAAFKDVRARMFVRFEEIAAYVASKGAGMDVYDALEKRELEQIKKLKSAVPERAHIQAYRAVLHRIGRAVQNCSEKTKQLFSSKVIEMGVFKNPSHLNNFIFNQKGAIYRSPFDRSRHAPYQLHADKLLKNDWLELLAEISATLMASESTEQMEDALRLERTRLQLQLSGLPDWEYPASLAKPDIEVEIQTALKMQLAKDTVTSDVLQRAFNLYSSVLSGLTFKLLRRSFSLKMRFSVADTTQLIYVPKVCDWAIPKQYLQAEGEIGIAARVVTESSPAKMVTEVEMKEPKALGHFMQQAPHDWYFDASLGGTQVAGRIVEKGKEVGKERKLVGYRMRGNSAYKTVLDKSLVGNTELSQCSMIIEIPYTQTVDADFRAQVQAGLPKVSINLPVKETITASNKDEQMLFDRFVAIDLGERGLGYAVFDAKTLELQESGHRPIKAITNLLNRTHHYEQRPNQRQKFQAKFNVNLSELRENTVGDVCHQINRICAYYNAFPVLEYMVPDRLDKQLKSVYESVTNRYIWSSTDAHKSARVQFWLGGETWEHPYLKSAKDKKPLVLSPGRGASGKGTSQTCSCCGRNPFDLIKDMKPRAKIAVVDGKAKLENSELKLFERNLESKDDMLARRHRNERAGMEQPLTPGNYTVDEIKALLRANLRRAPKNRRTKDTTVSEYHCVFSDCGKTMHADENAAVNIGGKFIADIEK

SEQ ID NO: 36 - protein, 1252 aa, Oleiphilus sp. HI0009
MTKLRHRQKKLTHDWAGSKKREVLGSNGKLQNPLLMPVKKGQVTEFRKAFSAYARATKGEMTDGRKNMFTHSFEPFKTKPSLHQCELADKAYQSLHSYLPGSLAHFLLSAHALGFRIFSKSGEATAFQASSKIEAYESKLASELACVDLSIQNLTISTLFNALTTSVRGKGEETSADPLIARFYTLLTGKPLSRDTQGPERDLAEVISRKIASSFGTWKEMTANPLQSLQFFEEELHALDANVSLSPAFDVLIKMNDLQGDLKNRTIVFDPDAPVFEYNAEDPADIIIKLTARYAKEAVIKNQNVGNYVKNAITTTNANGLGWLLNKGLSLLPVSTDDELLEFIGVERSHPSCHALIELIAQLEAPELFEKNVFSDTRSEVQGMIDSAVSNHIARLSSSRNSLSMDSEELERLIKSFQIHTPHCSLFIGAQSLSQQLESLPEALQSGVNSADILLGSTQYMLTNSLVEESIATYQRTLNRINYLSGVAGQINGAIKRKAIDGEKIHLPAAWSELISLPFIGQPVIDVESDLAHLKNQYQTLSNEFDTLISALQKNFDLNFNKALLNRTQHFEAMCRSTKKNALSKPEIVSYRDLLARLTSCLYRGSLVLRRAGIEVLKKHKIFESNSELREHVHERKHFVFVSPLDRKAKKLLRLTDSRPDLLHVIDEILQHDNLENKDRESLWLVRSGYLLAGLPDQLSSSFINLPIITQKGDRRLIDLIQYDQINRDAFVMLVTSAFKSNLSGLQYRANKQSFVVTRTLSPYLGSKLVYVPKDKDWLVPSQMFEGRFADILQSDYMVWKDAGRLCVIDTAKHLSNIKKSVFSSEEVLAFLRELPHRTFIQTEVRGLGVNVDGIAFNNGDIPSLKTFSNCVQVKVSRTNTSLVQTLNRWFEGGKVSPPSIQFERAYYKKDDQIHEDAAKRKIRFQMPATELVHASDDAGWTPSYLLGIDPGEYGMGLSLVSINNGEVLDSGFIHINSLINFASKKSNHQTKVVPRQQYKSPYANYLEQSKDSAAGDIAHILDRLIYKLNALPVFEALSGNSQSAADQVWTKVLSFYTWGDNDAQNSIRKQHWFGASHWDIKGMLRQPPTEKKPKPYIAFPGSQVSSYGNSQRCSCCGRNPIEQLREMAKDTSIKELKIRNSEIQLFDGTIKLFNPDPSTVIERRRHNLGPSRIPVADRTFKNISPSSLEFKELITIVSRSIRHSPEFIAKKRGIGSEYFCAYSDCNSSLNSEANAAANVAQKFQKQLFFEL

SEQ ID NO: 37 - DNA, 201 bp, Artificial Sequence
  CTEv4
tatcgataccgcggggatcctctagagtagaccacctcccctgcgagctaagctggacagccaatgacgactaagagagtgacatttttcactaagctaagacaggagggccgtcagagctactgcctaatccaaagacggctaaaagtgataaaaatgtatcactccaagttaagacaggcgcagcttccgagggatttg

SEQ ID NO: 38 - DNA, 110 bp, Artificial Sequence
  MmuMalat1 triple helix
gattcgtcagtagggttgtaaaggtttttcttttcctgagaaaacaaccttttgttttctcaggttttgctttttggcctttccctagctttaaaaaaaaaaaagcaaaa

SEQ ID NO: 39 - DNA, 170 bp, Artificial Sequence
  CTEv2
acctcccctgtgagctagactggacagccaatgacggagaagagagtgacatttctcactaacctaagacaggagggccgtcaaagctactgcctaatccaatgacgggtaatagtgacaagaaatgtatcactccactccaagacaggcgcagcctccgagggatgtgt

SEQ ID NO: 40 - DNA, 70 bp, Artificial Sequence
  CAE-m1
caccagactaagaacttagaacctcgctggaaaggaccctacaccgtcctgctgacaacccccaccgctc

SEQ ID NO: 41 - DNA, 226 bp, Artificial Sequence
  RTEm26-m1
ccgtggggtgcgaggctaagcactgcacagaggatagcttgctgttggcatcctgtggaaggcacgtctgattgcatgaaggttcagtgtcctagttcccttcccccaagaaaaacgacacgggagctggccaagacctctctgggtgatgagcctaagggatggttttgtgtagggcccctatgcttggcggctggggatcagacctctaccttcacccatgagg

SEQ ID NO: 42 - DNA, 599 bp, Artificial Sequence
  WPRE-m2
aaactaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccaaggctgctcgcctgtgttgcctcctggattctgcgcgggacgtccttctgctacgtcccttcggccctcaatccagcggaccttccttcccgcggcctgctgccggctctgcggcctcttccgcgtcttcgccttcgccctcggacgagtcggatctccctttgggccgcctccccgctatgc

SEQ ID NO: 43 - DNA, 99 bp, Artificial Sequence
  TAP-CTE-m1
cctgcttggccccgaggatggctggttagccaaagacgactaagattcctcagggaaggaacaagttaagacaggcacagtcgcagaggggccatcagg

SEQ ID NO: 44 - RNA, 169 nt, Artificial Sequence
  CTEv4
caccuccccugcgagcuaagcuggacagccaaugacgacuaagagagugacauuuuucacuaagcuaagacaggagggccgucagagcuacugccuaauccaaagacggcuaaaagugauaaaaauguaucacuccaaguuaagacaggcgcagcuuccgagggauuug

SEQ ID NO: 45 - DNA, 110 bp, Artificial Sequence; identical to SEQ ID NO: 38
  Malat1 triple helix

SEQ ID NO: 46 - DNA, 53 bp, Artificial Sequence
  XAP1 plus self-complementary flanking sequences
gggttgcacacgtctcgagggacgggcagggctgggcgagacgtgtgcaaccc

SEQ ID NO: 47 - DNA, 78 bp, Artificial Sequence
  xrRNA1
tagtcaggccagccggttaggctgccaccgaaggttggtagacggtgctgcctgcgaccaaccccaggaggactgggt

SEQ ID NO: 48 - DNA, 66 bp, Artificial Sequence
  xrRNA2
gtgtcagatcgcgaaagcgccacttcgccgaggagtgcaatctgtgaggccccaggaggactgggt

SEQ ID NO: 49 - DNA, 308 bp, Artificial Sequence
  xrRNA containing xrRNA 1 and xrRNA2 with linker sequences
acacttgcttttgacacaactgtgtttacttgcaatcccccaaaacagacagatagtcaggccagccggttaggctgccaccgaaggttggtagacggtgctgcctgcgaccaaccccaggaggactgggttaccaaagctgattctccacggttggaaagcctcccagaaccgtctcggaagaggagtccctgccaacaatggagatgaagcccgtgtcagatcgcgaaagcgccacttcgccgaggagtgcaatctgtgaggccccaggaggactgggtaaacagtcgacccgggcggccgcattt

SEQ ID NO: 50 - DNA, 232 bp, Artificial Sequence
  3′HCV-UTR
aacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagt

SEQ ID NO: 51 - protein, 353 aa, Artificial Sequence
  minimalGag-GCN4-PCP
MGARASVLSGGAQQAAADTGHSNQVSQNYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMGGSGGRMKQLEDKVEELLSKNYHLENEVARLKKLVGERGGSGGASSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRGGSAGGLQSRPEPTAPPEESFRSGVETTTPPQKQEPIDKELYPLTSLRSLFGNDPSSQ

SEQ ID NO: 52 - protein, 312 aa, Artificial Sequence
  minimalGag2-GCN4-PCP
MGARASVLSGGAQQAAADTGHSNQVSQNYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMGGSGGRMKQLEDKVEELLSKNYHLENEVARLKKLVGERGGSGGASSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRGGSAGGTASAPPPPYVG

SEQ ID NO: 53 - protein, 544 aa, Artificial Sequence
  dDBR1
MRVAVAGCCHGELDKIYETLALAERRGPGPVDLLLCCGDFQAVRNEADLRCMAVPPKYRHMQTFYRYYSGEKKAPVLTLFIGGNAEASNHLQELPYGGWVAPNIYYLGLAGVVKYRGVRIGGISGIFKSHDYRKGHFECPPYNSSTIRSIYHVRNIEVYKLKQLKQPIDIFLSHDWPRSIYHYGNKKQLLKTKSFFRQEVENNTLGSPAASELLEHLKPTYWFSAHLHVKFAALMQHQAKDKGQTARATKFLALDKCLPHRDFLQILEIEHDPSAPDYLEYDIEWLTILRATDDLINVTGRLWNMPENNGLHARWDYSATEEGMKEVLEKLNHDLKVPCNFSVTAACYDPSKPQTQMQLIHRINPQTTEFCAQLGIIDINVRLQKSKEEHHVCGEYEEQDDVESNDSGEDQSEYNTDTSALSSINPDEIMLDEEEDEDSIVSAHSGMNTPSVEPSDQASEFSASFSDVRILPGSMIVSSDDTVDSTIDREGKPGGTVESGNGEDLTKVPLKRLSDEHEPEQRKKIKRRNQAIYAAVDDDDDDAA

SEQ ID NO: 54 - protein, 554 aa, Artificial Sequence
  dDBR1-FLAG
MRVAVAGCCHGELDKIYETLALAERRGPGPVDLLLCCGDFQAVRNEADLRCMAVPPKYRHMQTFYRYYSGEKKAPVLTLFIGGNAEASNHLQELPYGGWVAPNIYYLGLAGVVKYRGVRIGGISGIFKSHDYRKGHFECPPYNSSTIRSIYHVRNIEVYKLKQLKQPIDIFLSHDWPRSIYHYGNKKQLLKTKSFFRQEVENNTLGSPAASELLEHLKPTYWFSAHLHVKFAALMQHQAKDKGQTARATKFLALDKCLPHRDFLQILEIEHDPSAPDYLEYDIEWLTILRATDDLINVTGRLWNMPENNGLHARWDYSATEEGMKEVLEKLNHDLKVPCNFSVTAACYDPSKPQTQMQLIHRINPQTTEFCAQLGIIDINVRLQKSKEEHHVCGEYEEQDDVESNDSGEDQSEYNTDTSALSSINPDEIMLDEEEDEDSIVSAHSGMNTPSVEPSDQASEFSASFSDVRILPGSMIVSSDDTVDSTIDREGKPGGTVESGNGEDLTKVPLKRLSDEHEPEQRKKIKRRNQAIYAAVDDDDDDAAGSDYKDDDDK